(19) United States
(12) Patent Application Publication
(10) Pub. No.: US 2025/0376685 A1
(43) Pub. Date: Dec. 11, 2025

(54) COMBINATION VECTORS AND METHODS FOR TREATING CANCER

(71) Applicant: American Gene Technologies International Inc., Rockville, MD (US)

(72) Inventors: Tyler LAHUSEN, Rockville, MD (US); Mei-Ling LIOU, Rockville, MD (US); Lingzhi XIAO, Rockville, MD (US); Haishan LI, Rockville, MD (US); Charles David PAUZA, Rockville, MD (US)

(21) Appl. No.: 19/232,291

(22) Filed: Jun. 9, 2025

Related U.S. Application Data
(62) Division of application No. 17/570,313, filed on Jan. 6, 2022, now Pat. No. 12,359,203.
Application No. 17/570,313 is a continuation of application No. 17/198,017, filed on Mar. 10, 2021, now Pat. No. 11,242,527.
Application No. 17/198,017 is a continuation of application No. 16/943,800, filed on Jul. 30, 2020, now Pat. No. 10,975,374.
Application No. 16/943,800 is a continuation of application No. 16/083,384, filed on Sep. 7, 2018, now Pat. No. 10,767,183.
(60) Provisional application No. 62/305,944, filed on Mar. 9, 2016.

(51) Int. Cl. C12N 15/113; A61P 35/00; C12N 15/62; C12N 15/85; C12N 15/86

(52) U.S. Cl. CPC C12N 15/1135; A61P 35/00; C12N 15/1137; C12N 15/1138; C12N 15/62; C12N 15/85; C12N 15/86; C12Y 205/01001; C12Y 205/0101; C12N 2320/31; C12N 2830/48

(57) ABSTRACT

A composition for treating cancer is disclosed. The composition includes a lentiviral particle and an aminobisphosphonate drug. The lentiviral particle is capable of infecting a target cell, such as a cancer cell, and includes an envelope protein optimized for targeting such target cell and a viral vector. The viral vector includes a small RNA optimized to target an FDPS mRNA sequence. The aminobisphosphonate drug includes zoledronic acid.

### CROSS-REFERENCE TO RELATED APPLICATIONS

This application is a divisional application of U.S. application Ser. No. 17/570,313 filed Jan. 6, 2022, now pending, which is a continuation application of U.S. application Ser. No. 17/198,017 filed Mar. 10, 2021, now issued as U.S. Pat. No. 11,242,527; which is a continuation application of U.S. application Ser. No. 16/943,800 filed Jul. 30, 2020, now issued as U.S. Pat. No. 10,975,374; which is a continuation application of U.S. application Ser. No. 16/083,384 filed Sep. 7, 2018, now issued as U.S. Pat. No. 10,767,183; which is a 35 USC § 371 National Stage application of International Application No. PCT/US2017/021639 filed Mar. 9, 2017, now expired; which claims the benefit under 35 USC § 119 (e) to U.S. Application Ser. No. 62/305,944 filed Mar. 9, 2016, now expired. The disclosure of each of the prior applications is considered part of and is incorporated by reference in the disclosure of this application.

### REFERENCE TO AN ELECTRONIC SEQUENCE LISTING

The contents of the electronic sequence listing (AGTI_008_05US_SeqList_ST26.xml; Size: 148,375 bytes; and Date of Creation: Jun. 6, 2025) are herein incorporated by reference in its entirety.

### BACKGROUND OF THE INVENTION

### Field of the Invention

Aspects of the present disclosure relate to using vectors to treat cancer. More specifically, aspects of the present disclosure relate to using vectors, including combination vectors, to treat cancer.

### Background Information

Cancer is a significant healthcare issue for the world's population. As an example, liver cancer in adult men is the fifth most frequently diagnosed cancer worldwide, and is the second leading cause of cancer-related death in the world. Numerous therapeutic strategies have been employed in an effort to effectively treat cancer. Traditional therapeutic approaches have revolved around the use of chemotherapy and radiation therapy.

Chemotherapy refers to the administration of one or more anti-cancer drugs and/or other agents to a cancer patient by various methods. Broadly, most chemotherapeutic drugs work by impairing mitosis (cell division), effectively targeting fast-dividing cells. However, other fast dividing cells such as those responsible for hair growth and for replacement of the intestinal epithelium (lining) are also affected. Because chemotherapy affects cell division, both normal and cancerous cells are susceptible to the cytotoxic effects of chemotherapeutic agents.

Radiation therapy refers to exposing a patient to high-energy radiation, including x-rays, gamma rays, and neutrons. This type of therapy includes without limitation external-beam therapy, internal radiation therapy, implant radiation, brachytherapy, systemic radiation therapy, and radiotherapy. External beam radiation may include three-dimensional conformal radiation therapy, intensity modulated radiation therapy, and conformal proton beam radiation therapy. In practice it is difficult to shield the nearby normal tissue from the cytotoxic effects of the radiation and still deliver a therapeutic dose. An additional complication of radiation is the induction of radiation resistant cells during the course of treatment. Thus, even the best radiotherapeutic techniques often result in incomplete tumor reduction and subsequent recurrence.

More recently, immunotherapeutic approaches have been employed in an attempt to harness the power of the host's immune system to treat cancer. For example, strategies have been employed to target cancer-associated antigens with host-based T cells that specifically recognize such antigens. For example, a recent approach has focused on the development and use of chimeric antigen receptor (CAR) T cells (also known as CAR-T cells). Possible side effects associated with CAR-T cell therapy include chemokine-release syndrome, B cell aplasia, and tumor lysis syndrome. Despite the development of these approaches, cancer remains a significant healthcare issue.

### SUMMARY OF THE INVENTION

In an aspect of the disclosure, a viral vector comprising a therapeutic cargo portion is disclosed. The therapeutic cargo portion includes at least one small RNA sequence that is capable of binding to at least one pre-determined complementary mRNA sequence, wherein the at least one complementary mRNA sequence comprises a FDPS mRNA sequence. In embodiments, the therapeutic cargo portion may further include a second small RNA sequence that is capable of binding to a second pre-determined complementary mRNA sequence, wherein the second pre-determined complementary mRNA sequence comprises a CD47 mRNA sequence or a cMyc mRNA sequence. In embodiments, the at least one small RNA sequence is under the control of a first promoter and the second small RNA sequence is under the control of a second promoter. In embodiments, the therapeutic cargo portion may further include a third small RNA sequence that is capable of binding to a third pre-determined complementary mRNA sequence, wherein the third pre-determined complementary mRNA sequence comprises a CD47 mRNA sequence or a cMyc mRNA sequence. In embodiments, the at least one small RNA sequence is under the control of a first promoter, the second small RNA sequence is under the control of a second promoter, and the third small RNA sequence is under the control of a third promoter. In embodiments, the small RNA sequences are under the control of a single promoter. In embodiments, the small RNA sequence is a microRNA (miRNA) or a short hairpin RNA (shRNA).

In another aspect, the small RNA sequence comprises a sequence having at least 80%, or at least 85%, or at least 90%, or at least 95% percent identity with a FDPS small RNA sequence comprising

(SEQ ID NO: 1)
GTCCTGGAGTACAATGCCATTCTCGAGAATGGCATTGTACTCCAGGACTT
TTT;
(SEQ ID NO: 2)
GCAGGATTTCGTTCAGCACTTCTCGAGAAGTGCTGAACGAAATCCTGCTT
TTT;
(SEQ ID NO: 3)
GCCATGTACATGGCAGGAATTCTCGAGAATTCCTGCCATGTACATGGCTT
TTT;
or
(SEQ ID NO: 4)
GCAGAAGGAGGCTGAGAAAGTCTCGAGACTTTCTCAGCCTCCTTCTGCTT
TTT.

In embodiments, the small RNA sequence is selected from SEQ ID NOs: 1, 2, 3, or 4.

In another aspect, the second small RNA sequence comprises a sequence having at least 80%, or at least 85%, or at least 90%, or at least 95% percent identity with a CD47 small RNA sequence comprising

(SEQ ID NO: 5)
GGTGAAACGATCATCGAGCCTCGAGGCTCGATGATCGTTTCACCTTTTT;
(SEQ ID NO: 6)
GCTACTGGCCTTGGTTTAACTCGAGTTAAACCAAGGCCAGTAGCTTTTT;
(SEQ ID NO: 7)
CCTCCTTCGTCATTGCCATCTCGAGATGGCAATGACGAAGGAGGTTTTT;
(SEQ ID NO: 8)
GCATGGCCCTCTTCTGATTCTCGAGAATCAGAAGAGGGCCATGCTTTTT;
or
(SEQ ID NO: 9)
GGTGAAACGATCATCGAGCTACTCGAGTAGCTCGATGATCGTTTCACCTT
TTT

or a cMyc small RNA sequence comprising

(SEQ ID NO: 10)
GCTTCACCAACAGGAACTATGCTCGAGCATAGTTCCTGTTGGTGAAGCTT
TT;
(SEQ ID NO: 11)
GCGAACACACAACGTCTTGGACTCGAGTCCAAGACGTTGTGTGTTCGCTT
TT;
(SEQ ID NO: 12)
GACATGGTGAACCAGAGTTTCCTCGAGGAAACTCTGGTTCACCATGTCTT
TTT;
(SEQ ID NO: 13)
GAGAATGTCAAGAGGCGAACACTCGAGTGTTCGCCTCTTGACATTCTCTT
TTT;
or
(SEQ ID NO: 14)
GCTCATTTCTGAAGAGGACTTCTCGAGAAGTCCTCTTCAGAAATGAGCTT
TTT.

In embodiments, the second small RNA sequence is selected from SEQ ID NOs: 5, 6, 7, 8, 9, 10, 11, 12, 13, or 14.

In another aspect, the third small RNA sequence comprises a sequence having at least 80%, or at least 85%, or at least 90%, or at least 95% percent identity with a CD47 small RNA sequence comprising SEQ ID NOs: 5, 6, 7, 8, or 9 or a cMyc small RNA sequence comprising SEQ ID NOs: 10, 11, 12, 13, or 14. In embodiments, the third small RNA sequence is selected from SEQ ID NOs: 5, 6, 7, 8, 9, 10, 11, 12, 13, or 14.

In another aspect, a viral vector comprising a therapeutic cargo portion is disclosed. The therapeutic cargo portion includes at least one small RNA sequence that is capable of binding to at least one pre-determined complementary mRNA sequence, wherein the at least one complementary mRNA sequence comprises a CD47 mRNA sequence. In embodiments, the therapeutic cargo portion further comprises a second small RNA sequence that is capable of binding to a second pre-determined complementary mRNA sequence, wherein the second pre-determined complementary mRNA sequence comprises a FDPS mRNA sequence or a cMyc mRNA sequence. In embodiments, the at least one small RNA sequence is under the control of a first promoter and the second small RNA sequence is under the control of a second promoter. In embodiments, the therapeutic cargo portion further comprises a third small RNA sequence that is capable of binding to a third pre-determined complementary mRNA sequence, wherein the third pre-determined complementary mRNA sequence comprises a FDPS mRNA sequence or a cMyc mRNA sequence. The small RNA sequence may be a miRNA or a shRNA. In embodiments, the at least one small RNA sequence is under the control of a first promoter, the second small RNA sequence is under the control of a second promoter, and the third small RNA sequence is under the control of a third promoter. In embodiments, the small RNA sequences are under the control of a single promoter.

In another aspect, the small RNA sequence comprises a sequence having at least 80%, or at least 85%, or at least 90%, or at least 95% percent identity with a CD47 small RNA sequence comprising SEQ ID NOs: 5, 6, 7, 8, or 9. In embodiments, the small RNA sequence is selected from SEQ ID NOs: 5, 6, 7, 8, or 9.

In another aspect, the second small RNA sequence comprises a sequence having at least 80%, or at least 85%, or at least 90%, or at least 95% percent identity with a FDPS small RNA sequence comprising SEQ ID NOs: 1, 2, 3, or 4 or a cMyc small RNA sequence comprising SEQ ID NOs: 10, 11, 12, 13, or 14. In embodiments, the second small RNA sequence is selected from SEQ ID NOs: 1, 2, 3, 4, 10, 11, 12, 13, or 14.

In another aspect, the third small RNA comprises a sequence having at least 80%, or at least 85%, or at least 90%, or at least 95% percent identity with a FDPS small RNA sequence comprising SEQ ID NOs: 1, 2, 3, or 4 or a cMyc small RNA sequence comprising SEQ ID NOs: 10, 11, 12, 13, or 14. In embodiments, the third small RNA sequence is selected from SEQ ID NOs: 1, 2, 3, 4, 10, 11, 12, 13, or 14.

In another aspect, a viral vector comprising a therapeutic cargo portion is disclosed. The therapeutic cargo portion comprises a first small RNA sequence that is capable of binding to a first pre-determined complementary mRNA sequence, and at least one additional small RNA sequence that is capable of binding to a second pre-determined complementary mRNA sequence, wherein the first pre-determined complementary mRNA sequence comprises a cMyc mRNA sequence, and the second pre-determined complementary sequence comprises a FDPS mRNA sequence or a CD47 mRNA sequence.

In another aspect, the therapeutic cargo portion further comprises a third small RNA sequence that is capable of binding to a third pre-determined complementary mRNA sequence, wherein the third pre-determined complementary mRNA sequence comprises a FDPS mRNA sequence or a CD47 mRNA sequence. In embodiments, the small RNA sequences are miRNAs or shRNAs. In embodiments, the first small RNA sequence is under the control of a first promoter, the second small RNA sequence is under the control of a second promoter, and the third small RNA sequence is under the control of a third promoter. In embodiments, the small RNA sequences are under the control of a single promoter.

In another aspect, the first small RNA sequence comprises a sequence having at least 80%, or at least 85%, or at least 90%, or at least 95% percent identity with a cMyc small RNA sequence comprising SEQ ID NOs: 10, 11, 12, 13, or 14. In embodiments, the first small RNA sequence is selected from SEQ ID NOs: 10, 11, 12, 13, or 14.

In another aspect, the at least one additional small RNA sequence comprises a sequence having at least 80%, or at least 85%, or at least 90%, or at least 95% percent identity with a FDPS small RNA sequence comprising SEQ ID NOs: 1, 2, 3, or 4 or a CD47 small RNA sequence comprising SEQ ID NOs: 5, 6, 7, 8, or 9. In embodiments, the at least one additional small RNA is selected from SEQ ID NOs: 1, 2, 3, 4, 5, 6, 7, 8 or 9.

In another aspect, the third small RNA sequence comprises a sequence having at least 80%, or at least 85%, or at least 90%, or at least 95% percent identity with a FDPS small RNA sequence comprising SEQ ID NOs: 1, 2, 3, or 4 or a CD47 small RNA sequence comprising SEQ ID NOs: 5, 6, 7, 8, or 9. In embodiments, the third small RNA sequence is selected from SEQ ID NOs: 1, 2, 3, 4, 5, 6, 7, 8 or 9.

In another aspect, the viral vector is a lentiviral vector. In another aspect, a lentiviral particle capable of infecting a target cell is disclosed. The lentiviral particle includes an envelope protein optimized for infecting the target cell, and the viral vector as described herein. In embodiments, the target cell is a tumor cell.

In another aspect, a composition is disclosed comprising the lentiviral particle as described herein, and an aminobisphosphonate drug. In embodiments, the aminobisphosphonate drug is zoledronic acid.

In another aspect, a method of treating cancer in a subject is disclosed. The method comprises administering to the subject a therapeutically effective amount of the composition as detailed herein.

In another aspect, a method of treating cancer in a subject is disclosed. The method comprises administering to the subject a therapeutically effective amount of the lentiviral particle as detailed herein, and a therapeutically effective amount of an aminobisphosphonate drug. In another aspect, a method of preventing cancer in a subject is disclosed. The method comprises administering to the subject a therapeutically effective amount of the lentiviral particle as detailed herein, and a therapeutically effective amount of an aminobisphosphonate drug. In embodiments, the foregoing steps are carried out simultaneously. In embodiments, a defined period of time elapses between the foregoing steps. In embodiments, the aminobisphosphonate drug is zoledronic acid. In embodiments, the therapeutically effective amount of the lentiviral particle comprises a plurality of single doses of the lentiviral particle. In embodiments, the therapeutically effective amount of the aminobisphosphonate drug comprises a single dose of the aminobisphosphonate drug.

Other aspects and advantages of the inventions described herein will become apparent from the following detailed description, taken in conjunction with the accompanying pc, which illustrate by way of example the aspects of the inventions.

### BRIEF DESCRIPTION OF THE DRAWINGS

FIG. 1 depicts an exemplary 3-vector lentiviral system in a circularized form.

FIG. 2 depicts an exemplary 4-vector lentiviral system in a circularized form.

FIGS. 3A-3C depict: (FIG. 3A) a linear map of a lentiviral vector encoding a FDPS shRNA targeting sequence; (FIG. 3B) a linear map of a lentiviral vector encoding a synthetic microRNA (miRNA) with a FDPS targeting sequence; and (FIG. 3C) a linear map of a lentiviral combination vector that encodes a synthetic microRNA (miRNA) with target sequences directed to cMyc, FDPS, and CD47 expression.

FIGS. 4A-4B depict: (FIG. 4A) relative expression levels of human FDPS mRNA in response to various shRNA constructs, as described herein; and (FIG. 4B) that lentiviral-delivered miR-based RNA interference inhibits FDPS expression.

FIGS. 5A-5B depict cytokine expression levels in human peripheral blood gamma delta T cells after exposure to (FIG. 5A) THP1 or (FIG. 5B) HepG2 cells that have been transduced with lentivirus to suppress FDPS.

FIG. 6 depicts percent specific lysis of THP-1 tumor cell line that was modified by lentiviral transduction to suppress FDPS then mixed with normal human gamma delta T cells under a variety of experimental conditions as described herein.

FIGS. 7A-7B depict: (FIG. 7A) relative expression levels of human CD47 mRNA in response to various shRNA constructs, as described herein; (FIG. 7B) that lentiviral-delivered miR-based RNA interference inhibits CD47 expression.

FIGS. 8A-8B depict: (FIG. 8A) the relative expression levels of human cMyc in response to various shRNA constructs, as described herein and (FIG. 8B) that lentiviral-delivered miR-based RNA interference inhibits cMyc expression.

FIG. 9 depicts a linear map of a lentiviral vector encoding a FDPS shRNA targeting sequence as used in Example 6 herein.

FIGS. 10A-10D depict the effect of zoledronic acid treatment of NOD/SCID mice implanted with PC3 cells transduced with LV-shFDPS or control LV as described herein. (FIG. 10A) depicts photographic data at day 8; (FIG. 10B) depicts photon intensity data at day 8; (FIG. 10C) depicts photographic data at day 22; and (FIG. 10D) depicts photon intensity data at day 22.

### DETAILED DESCRIPTION OF THE INVENTION

### Overview of the Disclosure

The present disclosure relates to therapeutic vectors and delivery of the same to cells. In embodiments, the therapeutic vectors target more than one mRNA target. In embodiments, the therapeutic vectors are provided with small RNAs, including short homology RNAs (shRNAs) or microRNAs (miRNAs) that target FDPS, thereby reducing expression levels of this enzyme. The therapeutic vectors include lentiviral vectors. The present disclosure demonstrates that targeting FDPS, in conjunction with treatment with an aminobisphosphonate drug, can effectively treat cancer.

### Definitions and Interpretation

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g.: Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual; Cold Spring Harbor Laboratory Press, Cold Spring Harbor, N.Y. (1998); and Coligan et al., Short Protocols in Protein Science, Wiley, John & Sons, Inc. (2003). Any enzymatic reactions or purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

As used in the description and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The terms “administration of” or “administering” an active agent should be understood to mean providing an active agent to the subject in need of treatment in a form that can be introduced into that individual's body in a therapeutically useful form and therapeutically effective amount.

As used herein, the term “combination vector” means a therapeutic vector that targets more than one mRNA. For example, a therapeutic vector that contains two shRNAs or two miRNAs directed towards two different mRNAs can be referred to as a “combination vector.”

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “expression,” “expressed,” or “encodes” refers to the process by which polynucleotides are transcribed into mRNA and/or the process by which the transcribed mRNA is subsequently being translated into peptides, polypeptides, or proteins. Expression may include splicing of the mRNA in a eukaryotic cell or other forms of post-transcriptional modification or post-translational modification.

The term “farnesyl diphosphate synthase” may also be referred to herein as FDPS, and may also be referred to herein as farnesyl pyrophosphate synthase or FPPS.

The term “gamma delta T cell” may also be referred to herein as a γδ T cell, or further as a GD T cell. The term “gamma delta T cell activation” refers to any measurable biological phenomenon associated with a gamma delta T cell that is representative of such T cell being activated. Non-limiting examples of such a biological phenomenon include an increase of cytokine production, changes in the qualitative or quantitative composition of cell surface proteins, an increase in T cell proliferation, and/or an increase in T cell effector function, such killing or a target cell or assisting another effector cell to kill a target cell. A target cell may be a cancer cell.

The terms “individual,” “subject,” and “patient” are used interchangeably herein, and refer to any individual mammal subject, e.g., bovine, canine, feline, equine, or human.

The term “LV” refers generally to “lentivirus.” As an example, reference to “LV-shFDPS” is reference to a lentivirus that expresses an shRNA that targets FDPS.

The term “miRNA” refers to a microRNA, and also may be referred to herein as “miR”.

The term “packaging cell line” refers to any cell line that can be used to express a lentiviral particle.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The percent identity between two nucleotide sequences can be determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules provided in the disclosure. BLAST protein searches can be performed with the XBLAST program, score=50, word length=3 to obtain amino acid sequences homologous to the protein molecules of the disclosure. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See www.ncbi.nlm.nih.gov.

As used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

As used herein, the term “SEQ ID NO” is synonymous with the term “Sequence ID No.”

As used herein, “small RNA” refers to non-coding RNA that are generally about 200 nucleotides or less in length and possess a silencing or interference function. In other embodiments, the small RNA is about 175 nucleotides or less, about 150 nucleotides or less, about 125 nucleotides or less, about 100 nucleotides or less, or about 75 nucleotides or less in length. Such RNAs include microRNA (miRNA), small interfering RNA (siRNA), double stranded RNA (dsRNA), and short hairpin RNA (shRNA). “Small RNA” of the disclosure should be capable of inhibiting or knocking-down gene expression of a target gene, generally through pathways that result in the destruction of the target gene mRNA.

The term “therapeutically effective amount” refers to a sufficient quantity of the active agents of the present disclosure, in a suitable composition, and in a suitable dosage form to treat or prevent the symptoms, progression, or onset of the complications seen in patients suffering from a given ailment, injury, disease, or condition. The therapeutically effective amount will vary depending on the state of the patient's condition or its severity, and the age, weight, etc., of the subject to be treated. A therapeutically effective amount can vary, depending on any of a number of factors, including, e.g., the route of administration, the condition of the subject, as well as other factors understood by those in the art.

As used herein, the term “therapeutic vector” includes, without limitation, reference to a lentiviral vector or an adeno-associated viral (AAV) vector. Additionally, as used herein with reference to the lentiviral vector system, the term “vector” is synonymous with the term “plasmid.” For example, the 3-vector and 4-vector systems, which include the 2-vector and 3-vector packaging systems, can also be referred to as 3-plasmid and 4-plasmid systems.

“A treatment” is intended to target the disease state and combat it, i.e., ameliorate or prevent the disease state. The particular treatment thus will depend on the disease state to be targeted and the current or future state of medicinal therapies and therapeutic approaches. A treatment may have associated toxicities.

The term “treatment” or “treating” generally refers to an intervention in an attempt to alter the natural course of the subject being treated and can be performed either for prophylaxis or during the course of clinical pathology. Desirable effects include, but are not limited to, preventing occurrence or recurrence of disease, alleviating symptoms, suppressing, diminishing or inhibiting any direct or indirect pathological consequences of the disease, ameliorating or palliating the disease state, and causing remission or improved prognosis.

### DESCRIPTION OF ASPECTS AND EMBODIMENTS OF THE DISCLOSURE

In an aspect of the disclosure, a viral vector comprising a therapeutic cargo portion is disclosed. The therapeutic cargo portion includes at least one small RNA sequence that is capable of binding to at least one pre-determined complementary mRNA sequence, wherein the at least one complementary mRNA sequence comprises a FDPS mRNA sequence. In embodiments, the therapeutic cargo portion may further include a second small RNA sequence that is capable of binding to a second pre-determined complementary mRNA sequence, wherein the second pre-determined complementary mRNA sequence comprises a CD47 mRNA sequence or a cMyc mRNA sequence. In embodiments, the therapeutic cargo portion may further include a third small RNA sequence that is capable of binding to a third pre-determined complementary mRNA sequence, wherein the third pre-determined complementary mRNA sequence comprises a CD47 mRNA sequence or a cMyc mRNA sequence. The small RNA sequence may be a microRNA (miRNA) or a short hairpin RNA (shRNA).

In another aspect, the small RNA sequence comprises a sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95% or greater percent identity with a FDPS small RNA sequence comprising SEQ ID NOs: 1, 2, 3, or 4. In embodiments, the small RNA sequence is selected from SEQ ID NOs: 1, 2, 3, or 4.

In another aspect, the second small RNA sequence comprises a sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95% or greater identity with a CD47 small RNA sequence comprising SEQ ID NOs: 5, 6, 7, 8 or 9 or a cMyc small RNA sequence comprising SEQ ID NOs: 10, 11, 12, 13, or 14. In embodiments, the second small RNA sequence is selected from SEQ ID NOs: 5, 6, 7, 8, 9, 10, 11, 12, 13, or 14.

In another aspect, the third small RNA sequence comprises a sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95% or greater identity with a CD47 small RNA sequence comprising SEQ ID NOs: 5, 6, 7, 8 or 9 or a cMyc small RNA sequence comprising SEQ ID NOs: 10, 11, 12, 13, or 14. In embodiments, the third small RNA sequence is selected from SEQ ID NOs: 5, 6, 7, 8, 9, 10, 11, 12, 13, or 14.

In another aspect, a viral vector comprising a therapeutic cargo portion is disclosed. The therapeutic cargo portion includes at least one small RNA sequence that is capable of binding to at least one pre-determined complementary mRNA sequence, wherein the at least one complementary mRNA sequence comprises a CD47 mRNA sequence. In embodiments, the therapeutic cargo portion further comprises a second small RNA sequence that is capable of binding to a second pre-determined complementary mRNA sequence, wherein the second pre-determined complementary mRNA sequence comprises a FDPS mRNA sequence or a cMyc mRNA sequence. In embodiments, the therapeutic cargo portion further comprises a third small RNA sequence that is capable of binding to a third pre-determined complementary mRNA sequence, wherein the third pre-determined complementary mRNA sequence comprises a FDPS mRNA sequence or a cMyc mRNA sequence. In embodiments, the small RNA sequence is a miRNA or a shRNA.

In another aspect, the small RNA sequence comprises a sequence having at least 80%, or at least 85%, or at least 90%, or at least 95% percent identity with a CD47 small RNA sequence comprising SEQ ID NOs: 5, 6, 7, 8 or 9. In embodiments, the small RNA sequence is selected from SEQ ID NOs: 5, 6, 7, 8 or 9.

In another aspect, the second small RNA sequence comprises a sequence having at least 80%, or at least 85%, or at least 90%, or at least 95% percent identity with a FDPS small RNA sequence comprising SEQ ID NOs: 1, 2, 3, or 4 or a cMyc small RNA sequence comprising SEQ ID NOs: 10, 11, 12, 13, or 14. In embodiments, the second small RNA sequence is selected from SEQ ID NOs: 1, 2, 3, 4, 10, 11, 12, 13, or 14.

In another aspect, the third small RNA comprises a sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95% or greater identity with a FDPS small RNA sequence comprising SEQ ID NOs: 1, 2, 3, or 4 or a cMyc small RNA sequence comprising SEQ ID NOs: 10, 11, 12, 13, or 14. In embodiments, the third small RNA sequence is selected from SEQ ID NOs: 1, 2, 3, 4, 10, 11, 12, 13, or 14.

In another aspect, a viral vector comprising a therapeutic cargo portion is disclosed. The therapeutic cargo portion comprises a first small RNA sequence that is capable of binding to a first pre-determined complementary mRNA sequence, and at least one additional small RNA sequence that is capable of binding to a second pre-determined complementary mRNA sequence, wherein the first pre-determined complementary mRNA sequence comprises a cMyc mRNA sequence, and the second pre-determined complementary sequence comprises a FDPS mRNA sequence or a CD47 mRNA sequence.

In another aspect, the therapeutic cargo portion further comprises a third small RNA sequence that is capable of binding to a third pre-determined complementary mRNA sequence, wherein the third pre-determined complementary mRNA sequence comprises a FDPS mRNA sequence or a CD47 mRNA sequence. In embodiments, the small RNA sequences are miRNAs or shRNAs.

In another aspect, the first small RNA sequence comprises a sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95% or greater identity with a cMyc small RNA sequence comprising SEQ ID NOs: 10, 11, 12, 13, or 14. In embodiments, the first small RNA sequence is selected from SEQ ID NOs: 10, 11, 12, 13, or 14.

In another aspect, the at least one additional small RNA sequence comprises a sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95% or greater percent identity with a FDPS small RNA sequence comprising SEQ ID NOs: 1, 2, 3, or 4 or a CD47 small RNA sequence comprising SEQ ID NOs: 5, 6, 7, 8 or 9. In embodiments, the at least one additional small RNA is selected from SEQ ID NOs: 1, 2, 3, 4, 5, 6, 7, 8, or 9.

In another aspect, the third small RNA sequence comprises a sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95% or greater identity with a FDPS small RNA sequence comprising SEQ ID NOs: 1, 2, 3, or 4 or a CD47 small RNA sequence comprising SEQ ID NOs: 5, 6, 7, 8 or 9. In embodiments, the third small RNA sequence is selected from SEQ ID NOs: 1, 2, 3, 4, 5, 6, 7, 8, or 9.

In another aspect, the small RNA sequences referred to herein can comprise a sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95% or greater identity with any of the miRNA sequences detailed herein, including: miR30 FDPS sequence #1 (SEQ ID NO: 53), miR30 FDPS sequence #2 (SEQ ID NO: 54), miR30 FDPS sequence #3 (SEQ ID NO: 55), miR155 FDPS sequence #1 (SEQ ID NO: 56), miR21 FDPS sequence #1 (SEQ ID NO: 57), miR185 FDPS sequence #1 (SEQ ID NO: 58), miR155 CD47 sequence #1 (SEQ ID NO: 82; miR155 CD47 target sequence #2 (SEQ ID NO: 66), miR155 CD47 target sequence #3 (SEQ ID NO: 67), miR155 CD47 target sequence #4 (SEQ ID NO: 68), miR21 cMyc sequence (SEQ ID NO: 83); or miR155 cMyc sequence (SEQ ID NO: 70).

In embodiments, the small RNA sequences can comprise any of the miRNA sequences detailed herein, including: miR30 FDPS sequence #1 (SEQ ID NO: 53), miR30 FDPS sequence #2 (SEQ ID NO: 54), miR30 FDPS sequence #3 (SEQ ID NO: 55), miR155 FDPS sequence #1 (SEQ ID NO: 56), miR21 FDPS sequence #1 (SEQ ID NO: 57), miR185 FDPS sequence #1 (SEQ ID NO: 58), miR155 CD47 sequence #1 (SEQ ID NO: 82; miR155 CD47 target sequence #2 (SEQ ID NO: 66), miR155 CD47 target sequence #3 (SEQ ID NO: 67), miR155 CD47 target sequence #4 (SEQ ID NO: 68), miR21 cMyc sequence (SEQ ID NO: 83); or miR155 cMyc sequence (SEQ ID NO: 70).

In another aspect, the viral vector is a lentiviral vector. In another aspect of the disclosure a lentiviral particle capable of infecting a target cell is disclosed. The lentiviral particle includes an envelope protein optimized for infecting the target cell; and the viral vector as described herein. In embodiments, the target cell is a tumor cell.

In another aspect, a composition is disclosed comprising the lentiviral particle as described herein, and an aminobisphosphonate drug. In embodiments, the aminobisphosphonate drug is zoledronic acid.

In another aspect of the disclosure, a method of treating cancer in a subject is disclosed. The method comprises administering to the subject a therapeutically effective amount of the composition as detailed herein.

In another aspect, a method of treating cancer in a subject is disclosed. The method comprises administering to the subject a therapeutically effective amount of the lentiviral particle as detailed herein; and a therapeutically effective amount of an aminobisphosphonate drug. In embodiments, the foregoing steps are carried out simultaneously. In embodiments, a defined period of time elapses between the foregoing steps. In embodiments, the aminobisphosphonate drug is zoledronic acid. In embodiments, the therapeutically effective amount of the lentiviral particle comprises a plurality of single doses of the lentiviral particle. In embodiments, the therapeutically effective amount of the aminobisphosphonate drugs comprises a single dose of the aminobisphosphonate drug.

Additional aspects of the present invention describe the development of multi-gene-targeting vectors for treatment of cancer, and, as a non-limiting example, for the treatment of hepatocellular carcinoma (“HCC”). These vectors address three concerns in respect of HCC therapy. Firstly, the therapeutic vectors may include inhibitory RNA constructs for reducing the expression of cMyc oncogene protein. The cMyc oncogene protein is responsible for tumorigenesis, tumor growth and immune evasion. The therapeutic vector may include more than just one inhibitory RNA construct for reducing cMyc expression. For example, in embodiments, combination vectors are specifically contemplated when cMyc is a target of the vector. Secondly, vectors have been developed (e.g., through inhibitory RNA constructs) to reduce the expression of farnesyl diphosphate synthase (“FDPS”). By reducing the levels of FDPS, tumor cells are modified, for example, to become stimulatory for gamma delta T cells. These gamma delta T cells are capable of cytotoxic killing of tumor cells. Thirdly, the vectors have been developed to reduce the expression (e.g., through inhibitory RNA constructs) of at least one other gene product. In certain embodiments, the at least one other gene product can be an immune checkpoint regulator. Examples of immune checkpoint regulators include, but are not limited to, programmed death-ligand 1 (PD-L1), galactosidase-binding soluble lectin 9 (LGALS9A), tumor necrosis factor receptor super family, member 14 (HVEM), V-set domain containing T cell activation inhibitor 1 (B7-H4), CD276 molecule (B7-H3), CD80 molecule (CD28LG1), and CD86 molecule (CD28LG2). In embodiments, the immune checkpoint regulator is PD-L1. By reducing expression cMyc, levels of PD-L1 are consequently decreased because cMyc is a positive regulator for expression of PD-L1 and other immune evasion genes including CD47, which are expressed in tumor cells. By decreasing the levels of CD47, tumor cell phagocytosis is increased leading to improved T cell responses through cross-presentation of tumor antigens on antigen-presenting cells. By decreasing PD-L1 and potentially other immune checkpoint inhibitory molecules, the efficiency of immune stimulation of T cells, including stimulation of gamma delta T cells, can be improved. While cMyc regulates PD-L1 levels, PD-L1 or other immune checkpoint regulators can be targeted directly using the therapeutic vectors described herein by generating shRNAs or miRNAs that are specifically directed to PD-L1 or the other selected immune checkpoint regulators.

In certain embodiments, the at least one other gene product can be a gene product that influences phagocytosis. For example, the at least one other gene product that influences phagocytosis can be CD47. By reducing the expression of CD47 the block to macrophage phagocytosis of tumor cells is removed. These two mechanisms combine to increase the efficiency and activity of acquired or innate immunity needed to treat or eliminate HCC.

The combination vectors disclosed herein are optimized such that the correct promoter has been selected to best match RNA processing system requirements. Additionally, the therapeutic cargo portion has been designed such that the miRNA or miRNAs are in a cluster so that processing of the first miRNA facilitates processing of the second miRNA and so on. The order of the miRNAs may be important to improve processing fidelity and associated rates so as to ensure that processing is not so rapid that genomic RNA for packaging into lentivirus particles is processed thus decreasing the efficiency of lentivirus manufacturing. Additionally, the combination vectors can be designed such that the therapeutic cargo portion includes multiple shRNAs under the control of discrete promoters.

### Cancer

The compositions and methods provided herein are used to treat cancer. A cell, tissue, or target may be a cancer cell, a cancerous tissue, harbor cancerous tissue, or be a subject or patient diagnosed or at risk of developing a disease or condition. In certain aspects, a cell may be an epithelial, an endothelial, a mesothelial, a glial, a stromal, or a mucosal cell. The cancer cell population can include, but is not limited to a brain, a neuronal, a blood, an endometrial, a meninges, an esophageal, a lung, a cardiovascular, a liver, a lymphoid, a breast, a bone, a connective tissue, a fat, a retinal, a thyroid, a glandular, an adrenal, a pancreatic, a stomach, an intestinal, a kidney, a bladder, a colon, a prostate, a uterine, an ovarian, a cervical, a testicular, a splenic, a skin, a smooth muscle, a cardiac muscle, or a striated muscle cell. In still a further aspect cancer includes, but is not limited to astrocytoma, acute myeloid leukemia, anaplastic large cell lymphoma, acute lymphoblastic leukemia, angiosarcoma, B-cell lymphoma, Burkitt's lymphoma, breast carcinoma, bladder carcinoma, carcinoma of the head and neck, cervical carcinoma, chronic lymphoblastic leukemia, chronic myeloid leukemia, colorectal carcinoma, endometrial carcinoma, esophageal squamous cell carcinoma, Ewing's sarcoma, fibrosarcoma, glioma, glioblastoma, gastrinoma, gastric carcinoma, hepatoblastoma, hepatocellular carcinoma, Kaposi's sarcoma, Hodgkin lymphoma, laryngeal squamous cell carcinoma, larynx carcinoma, leukemia, leiomyosarcoma, lipoma, liposarcoma, melanoma, mantle cell lymphoma, medulloblastoma, mesothelioma, myxofibrosarcoma, myeloid leukemia, mucosa-associated lymphoid tissue B cell lymphoma, multiple myeloma, high-risk myelodysplastic syndrome, nasopharyngeal carcinoma, neuroblastoma, neurofibroma, high-grade non-Hodgkin lymphoma, non-Hodgkin lymphoma, lung carcinoma, non-small cell lung carcinoma, ovarian carcinoma, esophageal carcinoma, osteosarcoma, pancreatic carcinoma, pheochromocytoma, prostate carcinoma, renal cell carcinoma, retinoblastoma, rhabdomyosarcoma, salivary gland tumor, Schwanomma, small cell lung cancer, squamous cell carcinoma of the head and neck, testicular tumor, thyroid carcinoma, urothelial carcinoma, and Wilm's tumor.

The compositions and methods provided herein are also used to treat NSCLC (non-small cell lung cancer), pediatric malignancies, cervical and other tumors caused or promoted by human papilloma virus (HPV), melanoma, Barrett's esophagus (pre-malignant syndrome), adrenal and skin cancers and auto immune, neoplastic cutaneous diseases.

### Therapeutic Vectors

The therapeutic vectors can be delivered via known transfection and/or transduction vectors, including but not limited to lentiviral vectors, adeno-associated virus, poxvirus, herpesvirus vectors, protein and/or lipid complexes, liposomes, micelles, and the like.

Viral vectors can be preferentially targeted to cell types that are useful for the disclosed methods (i.e., tumor cells or myeloid cells). Viral vectors can be used to transduce genes into target cells owing to specific virus envelope-host cell receptor interactions and viral mechanisms for gene expression. As a result, viral vectors have been used as vehicles for the transfer of genes into many different cell types including whole embryos, fertilized eggs, isolated tissue samples, tissue targets in situ, and cultured cell lines. The ability to introduce and express foreign genes in a cell is useful for the study of gene expression, and the elucidation of cell lineages as well as providing the potential for therapeutic interventions such as gene therapy, somatic cell reprogramming of induced pluripotent stem cells, and various types of immunotherapy. Viral components from viruses like Papovaviridae (e.g., bovine papillomavirus or BPV) or Herpesviridae (e.g., Epstein Barr Virus or EBV) or Hepadnaviridae (e.g., Hepatitis B Virus or HBV) or pox vectors including vaccinia may be used in the disclosed vectors.

Lentiviral vectors are a preferred type of vector for the disclosed compositions and methods, although the disclosure is not specifically limited to lentiviral vectors. Lentivirus is a genus of viruses that can deliver a significant amount of viral nucleic acid into a host cell. Lentiviruses are characterized as having a unique ability to infect/transduce non-dividing cells, and following transduction, lentiviruses integrate their nucleic acid into the host cell's chromosomes.

Infectious lentiviruses have three main genes coding for the virulence proteins gag, pol, and env, and two regulatory genes including tat and rev. Depending on the specific serotype and virus, there may be additional accessory genes that code for proteins involved in regulation, synthesis, and/or processing viral nucleic acids and other replicative functions.

Moreover, lentiviruses contain long terminal repeat (LTR) regions, which may be approximately 600 nt long. LTRs may be segmented into U3, R, and U5 regions. LTRs can mediate integration of retroviral DNA into the host chromosome via the action of integrase. Alternatively, without functioning integrase, the LTRs may be used to circularize the viral nucleic acid.

Viral proteins involved in early stages of lentivirus replication include reverse transcriptase and integrase. Reverse transcriptase is the virally encoded, RNA-dependent DNA polymerase. The enzyme uses a viral RNA genome as a template for the synthesis of a complementary DNA copy. Reverse transcriptase also has RNaseH activity for destruction of the RNA-template. Integrase binds both the viral cDNA generated by reverse transcriptase and the host DNA. Integrase processes the LTR before inserting the viral genome into the host DNA. Tat acts as a trans-activator during transcription to enhance initiation and elongation. The rev responsive element acts post-transcriptionally, regulating mRNA splicing and transport to the cytoplasm.

Viral vectors, in general, comprise glycoproteins and the various glycoproteins may provide specific affinities. For instance, VSVG peptides can increase transfection into myeloid cells. Alternatively, viral vectors can also have targeting moieties, such as antibodies, attached to their shell peptides. Targeting antibodies can be specific for antigens that are overexpressed on a tumor, for instance, like HER-2, PSA, CEA, M2-PK, and CA19-9. Other viral vector specificities are also known in the art and can be used to target particular populations of cells. For example, poxvirus vectors target to macrophages and dendritic cells.

With respect to the therapeutic vectors detailed herein, in aspects of the present disclosure, a miRNA or shRNA is under the control of a single promoter. In embodiments, when multiple miRNAs are present in the same therapeutic vector, the miRNAs are under the control of a single promoter, for example a Pol II promoter. In embodiments, the Pol II promoter is EF1-alpha or a CMV promoter.

In embodiments, when multiple shRNAs are present in the same therapeutic vector, the shRNAs are under the control of multiple promoters. For example, a first shRNA is under the control of a first promoter, a second shRNA is under the control of a second promoter, a third shRNA is under the control of a third promoter, and so on. In non-limiting embodiments, the promoters can be selected from H1 (SEQ ID NO: 15), U6 (SEQ ID NO: 16), or 7SK (SEQ ID NO: 17).

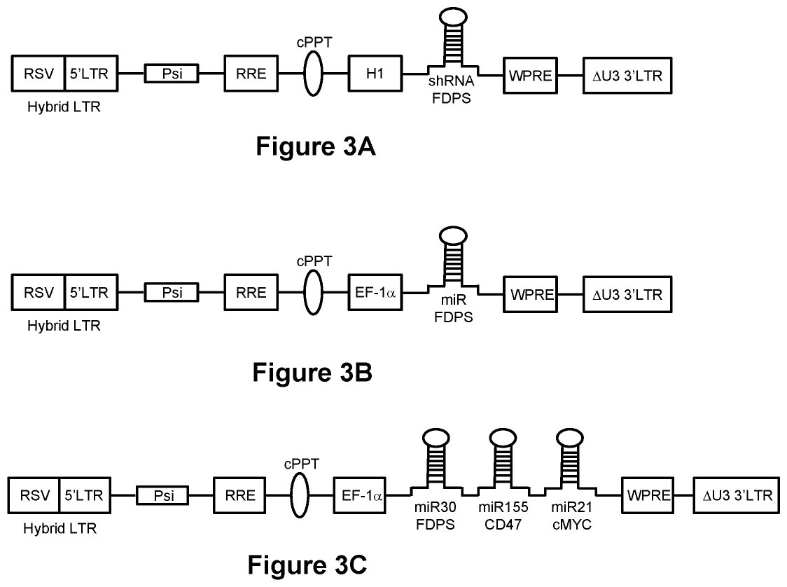

As depicted in FIG. 3C, a non-limiting example of a therapeutic vector includes a therapeutic cargo of three miRNA targeting cMyc, FDPS, and CD47 mRNA. As shown in Table 1 herein, alternate combinations of one to three miRNA sequences can be used in the final form of the therapeutic vector such that the therapeutic vector is a combination vector. While combinations of one to three miRNA sequences can be used in the final therapeutic vector, it is specifically contemplated that up to four, up to five, or up to six, or up to seven, or up to eight or more miRNA sequences could be used in the final therapeutic vector. Further the miRNA sequences may be sequential or randomly arranged (i.e., the first miRNA need not precede the second miRNA etc.). In addition to the combinations selected, all possible orders of miRNA from 5′ to 3′ end of the sense strand may be utilized for these lentiviral vectors. Vector components are not repeated for each miRNA combination. In developing the vectors containing miRNAs, shRNAs for the genes of interest are first used to prove that the gene of interest will work in the lentivirus construct; thereafter, and once shRNAs are proven to work (as described below), they are assembled into miRNA clusters as shown, for example, in FIG. 3C herein. The miRNAs preserve targeting sequences but have changes in their overall structure to become better suited for the miRNA processing pathway.

TABLE 1
Combinations of miRNA Sequences

| | | | |
|---|---|---|---|
| Vector 1 | | miR155FDPS | |
| Vector 2 | | | miR21CD47 |
| Vector 3 | miR30cMyc | | |
| Vector 4 | miR30cMyc | miR155FDPS | |
| Vector 5 | miR30cMyc | | miR21CD47 |
| Vector 6 | | miR155FDPS | miR21CD47 |
| Vector 7 | miR30cMyc | | miR21CD47 |
| Vector 8 | miR30cMyc | miR155FDPS | miR21CD47 |

Combination vectors can also be generated using shRNAs. However, in these circumstances discrete promoters need to be utilized for each target sequence, as is described herein.

### Lentiviral Vector System

A lentiviral virion (particle) is expressed by a vector system encoding the necessary viral proteins to produce a virion (viral particle). There is at least one vector containing a nucleic acid sequence encoding the lentiviral pol proteins necessary for reverse transcription and integration, operably linked to a promoter. In another embodiment, the pol proteins are expressed by multiple vectors. There is also a vector containing a nucleic acid sequence encoding the lentiviral gag proteins necessary for forming a viral capsid operably linked to a promoter. In an embodiment, this gag nucleic acid sequence is on a separate vector than at least some of the pol nucleic acid sequence. In another embodiment, the gag nucleic acid is on a separate vector from all the pol nucleic acid sequences that encode pol proteins.

Numerous modifications can be made to the vectors, which are used to create the particles to further minimize the chance of obtaining wild type revertants. These include, but are not limited to deletions of the U3 region of the LTR, tat deletions and matrix (MA) deletions.

The gag, pol and env vector(s) do not contain nucleotides from the lentiviral genome that package lentiviral RNA, referred to as the lentiviral packaging sequence.

The vector(s) forming the particle preferably do not contain a nucleic acid sequence from the lentiviral genome that expresses an envelope protein. Preferably, a separate vector that contains a nucleic acid sequence encoding an envelope protein operably linked to a promoter is used. This env vector also does not contain a lentiviral packaging sequence. In one embodiment the env nucleic acid sequence encodes a lentiviral envelope protein.

In another embodiment the envelope protein is not from the lentivirus, but from a different virus. The resultant particle is referred to as a pseudotyped particle. By appropriate selection of envelopes one can “infect” virtually any cell. For example, one can use an env gene that encodes an envelope protein that targets an endocytic compartment such as that of the influenza virus, VSV-G, alpha viruses (Semliki forest virus, Sindbis virus), arenaviruses (lymphocytic choriomeningitis virus), flaviviruses (tick-borne encephalitis virus, Dengue virus, hepatitis C virus, GB virus), rhabdoviruses (vesicular stomatitis virus, rabies virus), paramyxoviruses (mumps or measles) and orthomyxoviruses (influenza virus). Other envelopes that can preferably be used include those from Moloney Leukemia Virus such as MLV-E, MLV-A and GALV. These latter envelopes are particularly preferred where the host cell is a primary cell. Other envelope proteins can be selected depending upon the desired host cell. For example, targeting specific receptors such as a dopamine receptor can be used for brain delivery. Another target can be vascular endothelium. These cells can be targeted using a filovirus envelope. For example, the GP of Ebola, which by post-transcriptional modification become the GP, and GP₂ glycoproteins. In another embodiment, one can use different lentiviral capsids with a pseudotyped envelope (for example, FIV or SHIV [U.S. Pat. No. 5,654,195]). A SHIV pseudotyped vector can readily be used in animal models such as monkeys.

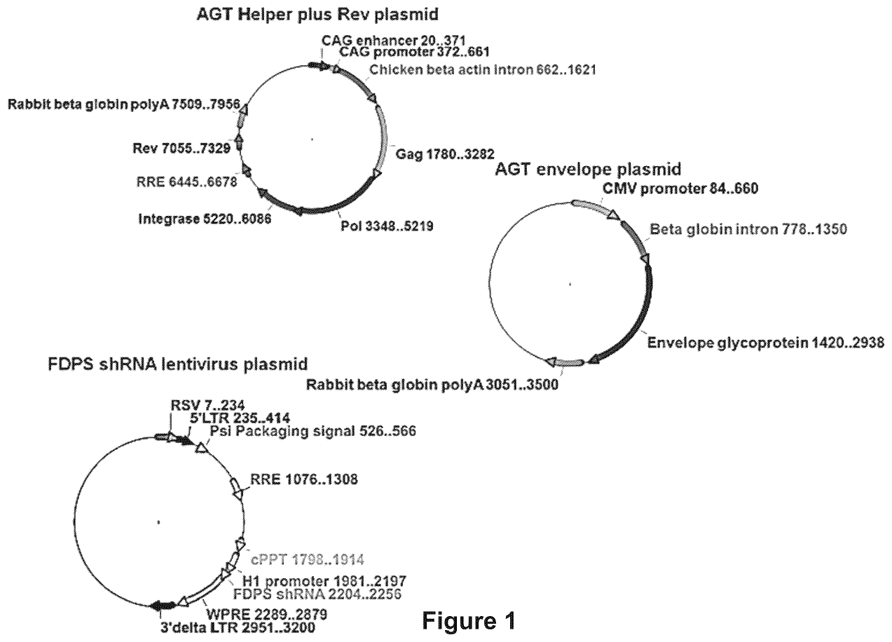

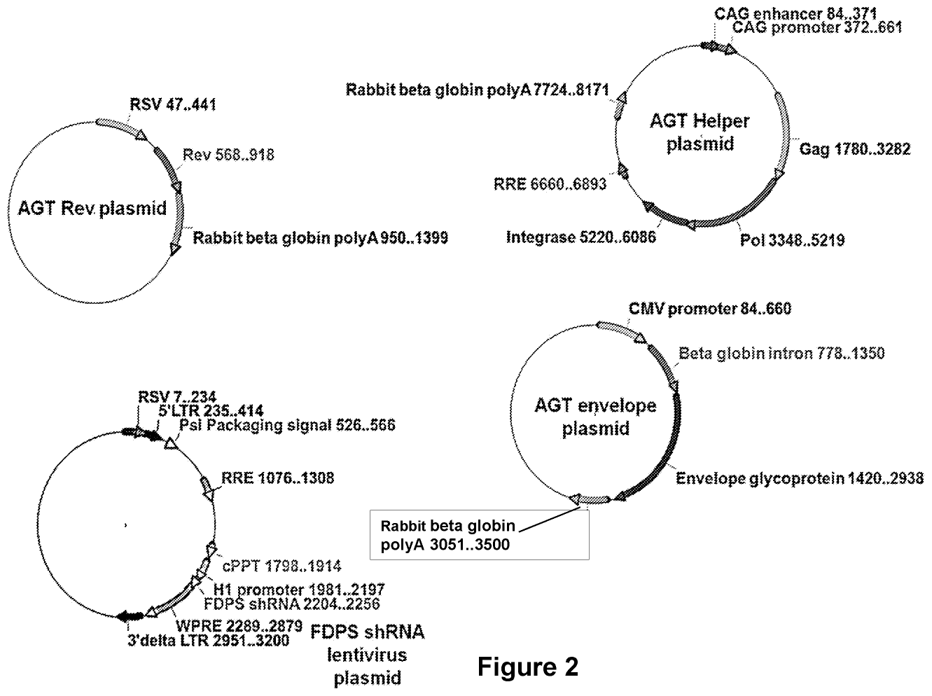

As detailed herein, a lentiviral vector system typically includes at least one helper plasmid comprising at least one of a gag, pol, or rev gene. Each of the gag, pol and rev genes may be provided on individual plasmids, or one or more genes may be provided together on the same plasmid. In one embodiment, the gag, pol, and rev genes are provided on the same plasmid (e.g., FIG. 1). In another embodiment, the gag and pol genes are provided on a first plasmid and the rev gene is provided on a second plasmid (e.g., FIG. 2). Accordingly, both 3-vector and 4-vector systems can be used to produce a lentivirus as described in the Examples section and elsewhere herein. The therapeutic vector, the envelope plasmid and at least one helper plasmid are transfected into a packaging cell line. A non-limiting example of a packaging cell line is the 293T/17 HEK cell line. When the therapeutic vector, the envelope plasmid, and at least one helper plasmid are transfected into the packaging cell line, a lentiviral particle is ultimately produced.

In another aspect, a lentiviral vector system for expressing a lentiviral particle is disclosed. The system includes a lentiviral vector as described herein; an envelope plasmid for expressing an envelope protein optimized for infecting a cell; and at least one helper plasmid for expressing gag, pol, and rev genes, wherein when the lentiviral vector, the envelope plasmid, and the at least one helper plasmid are transfected into a packaging cell line, a lentiviral particle is produced by the packaging cell line, wherein the lentiviral particle is capable of inhibiting the genes targeted by the shRNAs or miRNAs.

In another aspect, the therapeutic vector, can include the following elements: hybrid 5′ long terminal repeat (RSV/5′ LTR) (SEQ ID NOS: 74-75), Psi sequence (RNA packaging site) (SEQ ID NO: 76), RRE (Rev-response element) (SEQ ID NO: 77), cPPT (polypurine tract) (SEQ ID NO: 78), H1 promoter (SEQ ID NO: 15), FDPS shRNA (e.g., SEQ ID NOS: 1, 2, 3, 4 or variants thereof), Woodchuck Post-Transcriptional Regulatory Element (WPRE) (SEQ ID NO: 79), and 3′ Delta LTR (SEQ ID NO: 80). In another aspect, sequence variation, by way of substitution, deletion, addition, or mutation can be used to modify the sequences references herein.

In another aspect, and as detailed herein, a helper plasmid has been designed to include the following elements: CAG promoter (SEQ ID NO: 19); HIV component gag (SEQ ID NO: 21); HIV component pol (SEQ ID NO: 22); HIV Int (SEQ ID NO: 23); HIV RRE (SEQ ID NO: 24); and HIV Rev (SEQ ID NO: 25). In another aspect, the helper plasmid may be modified to include a first helper plasmid for expressing the gag and pol genes, and a second and separate plasmid for expressing the rev gene. In another aspect, sequence variation, by way of substitution, deletion, addition, or mutation can be used to modify the sequences references herein.

In another aspect, and as detailed herein, an envelope plasmid has been designed to include the following elements being from left to right: RNA polymerase II promoter (CMV) (SEQ ID NO: 27) and vesicular stomatitis virus G glycoprotein (VSV-G) (SEQ ID NO: 29). In another aspect, sequence variation, by way of substitution, deletion, addition, or mutation can be used to modify the sequences references herein.

In another aspect, the plasmids used for lentiviral packaging can be modified with similar elements and the intron sequences could potentially be removed without loss of vector function. For example, the following elements can replace similar elements in the plasmids that comprise the packaging system: Elongation Factor-1 (EF-1), phosphoglycerate kinase (PGK), and ubiquitin C (UbC) promoters can replace the CMV or CAG promoter. SV40 poly A and bGH poly A can replace the rabbit beta globin poly A. The HIV sequences in the helper plasmid can be constructed from different HIV strains or clades. The VSV-G glycoprotein can be substituted with membrane glycoproteins from feline endogenous virus (RD114), gibbon ape leukemia virus (GALV), Rabies (FUG), lymphocytic choriomeningitis virus (LCMV), influenza A fowl plague virus (FPV), Ross River alphavirus (RRV), murine leukemia virus 10A1 (MLV), or Ebola virus (EboV).

Of note, lentiviral packaging systems can be acquired commercially (e.g., Lenti-vpak packaging kit from OriGene Technologies, Inc., Rockville, MD), and can also be designed as described herein. Moreover, it is within the skill of a person skilled in the art to substitute or modify aspects of a lentiviral packaging system to improve any number of relevant factors, including the production efficiency of a lentiviral particle.

### Doses and Dosage Forms

The disclosed vector compositions allow for short, medium, or long-term expression of genes or sequences of interest and episomal maintenance of the disclosed vectors. Accordingly, dosing regimens may vary based upon the condition being treated and the method of administration.

In embodiments, vector compositions may be administered to a subject in need in varying doses. Specifically, a subject may be administered about ≥10⁶ infectious doses (where 1 dose is needed on average to transduce 1 target cell). More specifically, a subject may be administered about ≥10⁷, about ≥10⁸, about ≥10⁹, or about ≥10¹⁰ infectious doses, or any number of doses in-between these values. Upper limits of dosing will be determined for each disease indication, including a specific cancer type, and will depend on toxicity/safety profiles for each individual product or product lot.

Additionally, vector compositions of the present disclosure may be administered periodically, such as once or twice a day, or any other suitable time period. For example, vector compositions may be administered to a subject in need once a week, once every other week, once every three weeks, once a month, every other month, every three months, every six months, every nine months, once a year, every eighteen months, every two years, every thirty months, or every three years.

In embodiments, the disclosed vector compositions are administered as a pharmaceutical composition. In embodiments, the pharmaceutical composition can be formulated in a wide variety of dosage forms, including but not limited to nasal, pulmonary, oral, topical, or parenteral dosage forms for clinical application. Each of the dosage forms can comprise various solubilizing agents, disintegrating agents, surfactants, fillers, thickeners, binders, diluents such as wetting agents or other pharmaceutically acceptable excipients. The pharmaceutical composition can also be formulated for injection, insufflation, infusion, or intradermal exposure. For instance, an injectable formulation may comprise the disclosed vectors in an aqueous or non-aqueous solution at a suitable pH and tonicity.

The disclosed vector compositions may be administered to a subject via direct injection into a tumor site or at a site of infection. In some embodiments, the vectors can be administered systemically. In some embodiments, the vector compositions can be administered via guided cannulation to tissues immediately surrounding the sites of tumor or infection.

The disclosed vector compositions can be administered using any pharmaceutically acceptable method, such as intranasal, buccal, sublingual, oral, rectal, ocular, parenteral (intravenously, intradermally, intramuscularly, subcutaneously, intraperitoneally), pulmonary, intravaginal, locally administered, topically administered, topically administered after scarification, mucosally administered, via an aerosol, in semi-solid media such as agarose or gelatin, or via a buccal or nasal spray formulation.

Further, the disclosed vector compositions can be formulated into any pharmaceutically acceptable dosage form, such as a solid dosage form, tablet, pill, lozenge, capsule, liquid dispersion, gel, aerosol, pulmonary aerosol, nasal aerosol, ointment, cream, semi-solid dosage form, a solution, an emulsion, and a suspension. Further, the pharmaceutical composition may be a controlled release formulation, sustained release formulation, immediate release formulation, or any combination thereof. Further, the pharmaceutical composition may be a transdermal delivery system.

In embodiments, the pharmaceutical composition can be formulated in a solid dosage form for oral administration, and the solid dosage form can be powders, granules, capsules, tablets or pills. In embodiments, the solid dosage form can include one or more excipients such as calcium carbonate, starch, sucrose, lactose, microcrystalline cellulose or gelatin. In addition, the solid dosage form can include, in addition to the excipients, a lubricant such as talc or magnesium stearate. In some embodiments, the oral dosage form can be immediate release, or a modified release form. Modified release dosage forms include controlled or extended release, enteric release, and the like. The excipients used in the modified release dosage forms are commonly known to a person of ordinary skill in the art.

In embodiments, the pharmaceutical composition can be formulated as a sublingual or buccal dosage form. Such dosage forms comprise sublingual tablets or solution compositions that are administered under the tongue and buccal tablets that are placed between the cheek and gum.

In embodiments, the pharmaceutical composition can be formulated as a nasal dosage form. Such dosage forms of the present invention comprise solution, suspension, and gel compositions for nasal delivery.

In embodiments, the pharmaceutical composition can be formulated in a liquid dosage form for oral administration, such as suspensions, emulsions or syrups. In embodiments, the liquid dosage form can include, in addition to commonly used simple diluents such as water and liquid paraffin, various excipients such as humectants, sweeteners, aromatics or preservatives. In embodiments, the composition can be formulated to be suitable for administration to a pediatric patient.

In embodiments, the pharmaceutical composition can be formulated in a dosage form for parenteral administration, such as sterile aqueous solutions, suspensions, emulsions, non-aqueous solutions or suppositories. In embodiments, the solutions or suspensions can include propyleneglycol, polyethyleneglycol, vegetable oils such as olive oil or injectable esters such as ethyl oleate.

The dosage of the pharmaceutical composition can vary depending on the patient's weight, age, gender, administration time and mode, excretion rate, and the severity of disease.

In embodiments, the treatment of cancer is accomplished by guided direct injection of the disclosed vector constructs into tumors, using needle, or intravascular cannulation. In embodiments, the vectors compositions are administered into the cerebrospinal fluid, blood or lymphatic circulation by venous or arterial cannulation or injection, intradermal delivery, intramuscular delivery or injection into a draining organ near the site of disease.

The following examples are given to illustrate aspects of the present invention. It should be understood, however, that the invention is not to be limited to the specific conditions or details described in these examples. All printed publications referenced herein are specifically incorporated by reference.

### EXAMPLES

### Example 1

### Development of a Lentiviral Vector System

A lentiviral vector system was developed as summarized in FIG. 1 (circularized form). Lentiviral particles were produced in 293T/17 HEK cells (purchased from American Type Culture Collection, Manassas, VA) following transfection with the therapeutic vector, the envelope plasmid, and the helper plasmid. The transfection of 293T/17 HEK cells, which produced functional viral particles, employed the reagent Poly(ethylenimine) (PEI) to increase the efficiency of plasmid DNA uptake. The plasmids and DNA were initially added separately in culture medium without serum in a ratio of 3:1 (mass ratio of PEI to DNA). After 2-3 days, cell medium was collected and lentiviral particles were purified by high-speed centrifugation and/or filtration followed by anion-exchange chromatography. The concentration of lentiviral particles can be expressed in terms of transducing units/ml (TU/ml). The determination of TU was accomplished by measuring HIV p24 levels in culture fluids (p24 protein is incorporated into lentiviral particles), measuring the number of viral DNA copies per transduced cell by quantitative PCR, or by infecting cells and using light (if the vectors encode luciferase or fluorescent protein markers).

As mentioned above, a 3-vector system (i.e., which includes a 2-vector lentiviral packaging system) was designed for the production of lentiviral particles. A schematic of the 3-vector system is shown in FIG. 1. Briefly, and with reference to FIG. 1, the top-most vector is a helper plasmid, which, in this case, includes Rev. The vector appearing in the middle of FIG. 1 is the envelope plasmid. The bottom-most vector is the therapeutic vector, as described herein.

Referring to FIG. 1, the Helper plus Rev plasmid includes a CAG enhancer (SEQ ID NO: 18); a CAG promoter (SEQ ID NO: 19); a chicken beta actin intron (SEQ ID NO: 20); a HIV gag (SEQ ID NO: 21); a HIV Pol (SEQ ID NO: 22); a HIV Int (SEQ ID NO: 23); a HIV RRE (SEQ ID NO: 24); a HIV Rev (SEQ ID NO: 25); and a rabbit beta globin poly A (SEQ ID NO: 26).

The Envelope plasmid includes a CMV promoter (SEQ ID NO: 27); a beta globin intron (SEQ ID NO: 28); a VSV-G (SEQ ID NO: 29); and a rabbit beta globin poly A (SEQ ID NO: 30).

Synthesis of a 3-Vector System, which Includes a 2-Vector Lentiviral Packaging System, Consisting of Helper (Plus Rev) and Envelope Plasmids.

### Materials and Methods:

### Construction of the Helper Plasmid:

The helper plasmid was constructed by initial PCR amplification of a DNA fragment from the pNL4-3 HIV plasmid (NIH Aids Reagent Program) containing Gag, Pol, and Integrase genes. Primers were designed to amplify the fragment with EcoRI and NotI restriction sites which could be used to insert at the same sites in the pCDNA3 plasmid (Invitrogen). The forward primer was (5′-TAAGCAGAATTC ATGAATTTGCCAGGAAGAT-3′) (SEQ ID NO: 31) and reverse primer was (5′-CCATACAATGAATGGACACTAGGCGGCCGCACGAAT-3′) (SEQ ID NO: 32).

The sequence for the Gag, Pol, Integrase fragment was as follows:

(SEQ ID NO: 33)
GAATTCATGAATTTGCCAGGAAGATGGAAACCAAAAATGATAGGGGGAATTGGAGGTTTT
ATCAAAGTAAGACAGTATGATCAGATACTCATAGAAATCTGCGGACATAAAGCTATAGGT
ACAGTATTAGTAGGACCTACACCTGTCAACATAATTGGAAGAAATCTGTTGACTCAGATT
GGCTGCACTTTAAATTTTCCCATTAGTCCTATTGAGACTGTACCAGTAAAATTAAAGCCA
GGAATGGATGGCCCAAAAGTTAAACAATGGCCATTGACAGAAGAAAAAATAAAAGCATTA
GTAGAAATTTGTACAGAAATGGAAAAGGAAGGAAAAATTTCAAAAATTGGGCCTGAAAAT
CCATACAATACTCCAGTATTTGCCATAAAGAAAAAAGACAGTACTAAATGGAGAAAATTA
GTAGATTTCAGAGAACTTAATAAGAGAACTCAAGATTTCTGGGAAGTTCAATTAGGAATA
CCACATCCTGCAGGGTTAAAACAGAAAAAATCAGTAACAGTACTGGATGTGGGCGATGCA
TATTTTTCAGTTCCCTTAGATAAAGACTTCAGGAAGTATACTGCATTTACCATACCTAGT
ATAAACAATGAGACACCAGGGATTAGATATCAGTACAATGTGCTTCCACAGGGATGGAAA
GGATCACCAGCAATATTCCAGTGTAGCATGACAAAAATCTTAGAGCCTTTTAGAAAACAA
AATCCAGACATAGTCATCTATCAATACATGGATGATTTGTATGTAGGATCTGACTTAGAA
ATAGGGCAGCATAGAACAAAAATAGAGGAACTGAGACAACATCTGTTGAGGTGGGGATTT
ACCACACCAGACAAAAAACATCAGAAAGAACCTCCATTCCTTTGGATGGGTTATGAACTC
CATCCTGATAAATGGACAGTACAGCCTATAGTGCTGCCAGAAAAGGACAGCTGGACTGTC
AATGACATACAGAAATTAGTGGGAAAATTGAATTGGGCAAGTCAGATTTATGCAGGGATT
AAAGTAAGGCAATTATGTAAACTTCTTAGGGGAACCAAAGCACTAACAGAAGTAGTACCA
CTAACAGAAGAAGCAGAGCTAGAACTGGCAGAAAACAGGGAGATTCTAAAAGAACCGGTA
CATGGAGTGTATTATGACCCATCAAAAGACTTAATAGCAGAAATACAGAAGCAGGGGCAA
GGCCAATGGACATATCAAATTTATCAAGAGCCATTTAAAAATCTGAAAACAGGAAAGTAT
GCAAGAATGAAGGGTGCCCACACTAATGATGTGAAACAATTAACAGAGGCAGTACAAAAA
ATAGCCACAGAAAGCATAGTAATATGGGGAAAGACTCCTAAATTTAAATTACCCATACAA
AAGGAAACATGGGAAGCATGGTGGACAGAGTATTGGCAAGCCACCTGGATTCCTGAGTGG
GAGTTTGTCAATACCCCTCCCTTAGTGAAGTTATGGTACCAGTTAGAGAAAGAACCCATA
ATAGGAGCAGAAACTTTCTATGTAGATGGGGCAGCCAATAGGGAAACTAAATTAGGAAAA
GCAGGATATGTAACTGACAGAGGAAGACAAAAAGTTGTCCCCCTAACGGACACAACAAAT
CAGAAGACTGAGTTACAAGCAATTCATCTAGCTTTGCAGGATTCGGGATTAGAAGTAAAC
ATAGTGACAGACTCACAATATGCATTGGGAATCATTCAAGCACAACCAGATAAGAGTGAA
TCAGAGTTAGTCAGTCAAATAATAGAGCAGTTAATAAAAAAGGAAAAAGTCTACCTGGCA
TGGGTACCAGCACACAAAGGAATTGGAGGAAATGAACAAGTAGATAAATTGGTCAGTGCT
GGAATCAGGAAAGTACTATTTTTAGATGGAATAGATAAGGCCCAAGAAGAACATGAGAAA
TATCACAGTAATTGGAGAGCAATGGCTAGTGATTTTAACCTACCACCTGTAGTAGCAAAA
GAAATAGTAGCCAGCTGTGATAAATGTCAGCTAAAAGGGGAAGCCATGCATGGACAAGTA
GACTGTAGCCCAGGAATATGGCAGCTAGATTGTACACATTTAGAAGGAAAAGTTATCTTG
GTAGCAGTTCATGTAGCCAGTGGATATATAGAAGCAGAAGTAATTCCAGCAGAGACAGGG
CAAGAAACAGCATACTTCCTCTTAAAATTAGCAGGAAGATGGCCAGTAAAAACAGTACAT
ACAGACAATGGCAGCAATTTCACCAGTACTACAGTTAAGGCCGCCTGTTGGTGGGCGGGG
ATCAAGCAGGAATTTGGCATTCCCTACAATCCCCAAAGTCAAGGAGTAATAGAATCTATG
AATAAAGAATTAAAGAAAATTATAGGACAGGTAAGAGATCAGGCTGAACATCTTAAGACA
GCAGTACAAATGGCAGTATTCATCCACAATTTTAAAAGAAAAGGGGGGATTGGGGGGTAC
AGTGCAGGGGAAAGAATAGTAGACATAATAGCAACAGACATACAAACTAAAGAATTACAA
AAACAAATTACAAAAATTCAAAATTTTCGGGTTTATTACAGGGACAGCAGAGATCCAGTT
TGGAAAGGACCAGCAAAGCTCCTCTGGAAAGGTGAAGGGGCAGTAGTAATACAAGATAAT
AGTGACATAAAAGTAGTGCCAAGAAGAAAAGCAAAGATCATCAGGGATTATGGAAAACAG
ATGGCAGGTGATGATTGTGTGGCAAGTAGACAGGATGAGGATTAA

Next, a DNA fragment containing the Rev, RRE, and rabbit beta globin poly A sequence with XbaI and XmaI flanking restriction sites was synthesized by MWG Operon. The DNA fragment was then inserted into the plasmid at the XbaI and XmaI restriction sites The DNA sequence was as follows:

(SEQ ID NO: 34)
TCTAGAATGGCAGGAAGAAGCGGAGACAGCGACGAAGAGCTCATCAGAACAGTCAGACTC
ATCAAGCTTCTCTATCAAAGCAACCCACCTCCCAATCCCGAGGGGACCCGACAGGCCCGA
AGGAATAGAAGAAGAAGGTGGAGAGAGAGACAGAGACAGATCCATTCGATTAGTGAACGG
ATCCTTGGCACTTATCTGGGACGATCTGCGGAGCCTGTGCCTCTTCAGCTACCACCGCTT
GAGAGACTTACTCTTGATTGTAACGAGGATTGTGGAACTTCTGGGACGCAGGGGGTGGGA
AGCCCTCAAATATTGGTGGAATCTCCTACAATATTGGAGTCAGGAGCTAAAGAATAGAGG
AGCTTTGTTCCTTGGGTTCTTGGGAGCAGCAGGAAGCACTATGGGCGCAGCGTCAATGAC
GCTGACGGTACAGGCCAGACAATTATTGTCTGGTATAGTGCAGCAGCAGAACAATTTGCT
GAGGGCTATTGAGGCGCAACAGCATCTGTTGCAACTCACAGTCTGGGGCATCAAGCAGCT
CCAGGCAAGAATCCTGGCTGTGGAAAGATACCTAAAGGATCAACAGCTCCTAGATCTTTT
TCCCTCTGCCAAAAATTATGGGGACATCATGAAGCCCCTTGAGCATCTGACTTCTGGCTA
ATAAAGGAAATTTATTTTCATTGCAATAGTGTGTTGGAATTTTTTGTGTCTCTCACTCGG
AAGGACATATGGGAGGGCAAATCATTTAAAACATCAGAATGAGTATTTGGTTTAGAGTTT
GGCAACATATGCCATATGCTGGCTGCCATGAACAAAGGTGGCTATAAAGAGGTCATCAGT
ATATGAAACAGCCCCCTGCTGTCCATTCCTTATTCCATAGAAAAGCCTTGACTTGAGGTT
AGATTTTTTTTATATTTTGTTTTGTGTTATTTTTTTCTTTAACATCCCTAAAATTTTCCT
TACATGTTTTACTAGCCAGATTTTTCCTCCTCTCCTGACTACTCCCAGTCATAGCTGTCC
CTCTTCTCTTATGAAGATCCCTCGACCTGCAGCCCAAGCTTGGCGTAATCATGGTCATAG
CTGTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCGGATCCGCATCTCAATTAGT
CAGCAACCATAGTCCCGCCCCTAACTCCGCCCATCCCGCCCCTAACTCCGCCCAGTTCCG
CCCATTCTCCGCCCCATGGCTGACTAATTTTTTTTATTTATGCAGAGGCCGAGGCCGCCT
CGGCCTCTGAGCTATTCCAGAAGTAGTGAGGAGGCTTTTTTGGAGGCCTAGGCTTTTGCA
AAAAGCTAACTTGTTTATTGCAGCTTATAATGGTTACAAATAAAGCAATAGCATCACAAA
TTTCACAAATAAAGCATTTTTTTCACTGCATTCTAGTTGTGGTTTGTCCAAACTCATCAA
TGTATCTTATCAGCGGCCGCCCCGGG.

Finally, the CMV promoter of pCDNA3.1 was replaced with the CAG enhancer/promoter plus a chicken beta actin intron sequence. A DNA fragment containing the CAG enhancer/promoter/intron sequence with MluI and EcoRI flanking restriction sites was synthesized by MWG Operon. The DNA fragment was then inserted into the plasmid at the MluI and EcoRI restriction sites. The DNA sequence was as follows:

(SEQ ID NO: 35)
ACGCGTTAGTTATTAATAGTAATCAATTACGGGGTCATTAGTTCATAGCCCATATATGGA
GTTCCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCATTG
ACGTCAATGGGTGGACTATTTACGGTAAACTGCCCACTTGGCAGTACATCAAGTGTATCA
TATGCCAAGTACGCCCCCTATTGACGTCAATGACGGTAAATGGCCCGCCTGGCATTATGC
CCAGTACATGACCTTATGGGACTTTCCTACTTGGCAGTACATCTACGTATTAGTCATCGC
TATTACCATGGGTCGAGGTGAGCCCCACGTTCTGCTTCACTCTCCCCATCTCCCCCCCCT
CCCCACCCCCAATTTTGTATTTATTTATTTTTTAATTATTTTGTGCAGCGATGGGGGCGG
GGGGGGGGGGGGCGCGCGCCAGGCGGGGCGGGGCGGGGCGAGGGGCGGGGCGGGGCGAGG
CGGAGAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGCGGGCGGGAGTCGCTGCG
TTGCCTTCGCCCCGTGCCCCGCTCCGCGCCGCCTCGCGCCGCCCGCCCCGGCTCTGACTG
ACCGCGTTACTCCCACAGGTGAGCGGGCGGGACGGCCCTTCTCCTCCGGGCTGTAATTAG
CGCTTGGTTTAATGACGGCTCGTTTCTTTTCTGTGGCTGCGTGAAAGCCTTAAAGGGCTC
CGGGAGGGCCCTTTGTGCGGGGGGGAGCGGCTCGGGGGGTGCGTGCGTGTGTGTGTGCGT
GGGGAGCGCCGCGTGCGGCCCGCGCTGCCCGGCGGCTGTGAGCGCTGCGGGCGCGGCGCG
GGGCTTTGTGCGCTCCGCGTGTGCGCGAGGGGAGCGCGGCCGGGGGCGGTGCCCCGCGGT
GCGGGGGGGCTGCGAGGGGAACAAAGGCTGCGTGCGGGGTGTGTGCGTGGGGGGGTGAGC
AGGGGGTGTGGGCGCGGCGGTCGGGCTGTAACCCCCCCCTGCACCCCCCTCCCCGAGTTG
CTGAGCACGGCCCGGCTTCGGGTGCGGGGCTCCGTGCGGGGCGTGGCGCGGGGCTCGCCG
TGCCGGGCGGGGGGTGGCGGCAGGTGGGGGTGCCGGGCGGGGCGGGGCCGCCTCGGGCCG
GGGAGGGCTCGGGGGAGGGGCGCGGCGGCCCCGGAGCGCCGGCGGCTGTCGAGGCGCGGC
GAGCCGCAGCCATTGCCTTTTATGGTAATCGTGCGAGAGGGCGCAGGGACTTCCTTTGTC
CCAAATCTGGCGGAGCCGAAATCTGGGAGGCGCCGCCGCACCCCCTCTAGCGGGCGCGGG
CGAAGCGGTGCGGCGCCGGCAGGAAGGAAATGGGCGGGGAGGGCCTTCGTGCGTCGCCGC
GCCGCCGTCCCCTTCTCCATCTCCAGCCTCGGGGCTGCCGCAGGGGGACGGCTGCCTTCG
GGGGGGACGGGGCAGGGCGGGGTTCGGCTTCTGGCGTGTGACCGGCGGGAATTC.

### Construction of the VSV-G Envelope Plasmid

The vesicular stomatitis Indiana virus glycoprotein (VSV-G) sequence was synthesized by MWG Operon with flanking EcoRI restriction sites. The DNA fragment was then inserted into the pCDNA3.1 plasmid (Invitrogen) at the EcoRI restriction site and the correct orientation was determined by sequencing using a CMV specific primer. The DNA sequence was as follows:

(SEQ ID NO: 29)
GAATTCATGAAGTGCCTTTTGTACTTAGCCTTTTTATTCATTGGGGTGAAT
TGCAAGTTCACCATAGTTTTTCCACACAACCAAAAAGGAAACTGGAAAAAT
GTTCCTTCTAATTACCATTATTGCCCGTCAAGCTCAGATTTAAATTGGCAT
AATGACTTAATAGGCACAGCCTTACAAGTCAAAATGCCCAAGAGTCACAAG
GCTATTCAAGCAGACGGTTGGATGTGTCATGCTTCCAAATGGGTCACTACT
TGTGATTTCCGCTGGTATGGACCGAAGTATATAACACATTCCATCCGATCC
TTCACTCCATCTGTAGAACAATGCAAGGAAAGCATTGAACAAACGAAACAA
GGAACTTGGCTGAATCCAGGCTTCCCTCCTCAAAGTTGTGGATATGCAACT
GTGACGGATGCCGAAGCAGTGATTGTCCAGGTGACTCCTCACCATGTGCTG
GTTGATGAATACACAGGAGAATGGGTTGATTCACAGTTCATCAACGGAAAA
TGCAGCAATTACATATGCCCCACTGTCCATAACTCTACAACCTGGCATTCT
GACTATAAGGTCAAAGGGCTATGTGATTCTAACCTCATTTCCATGGACATC
ACCTTCTTCTCAGAGGACGGAGAGCTATCATCCCTGGGAAAGGAGGGCACA
GGGTTCAGAAGTAACTACTTTGCTTATGAAACTGGAGGCAAGGCCTGCAAA
ATGCAATACTGCAAGCATTGGGGAGTCAGACTCCCATCAGGTGTCTGGTTC
GAGATGGCTGATAAGGATCTCTTTGCTGCAGCCAGATTCCCTGAATGCCCA
GAAGGGTCAAGTATCTCTGCTCCATCTCAGACCTCAGTGGATGTAAGTCTA
ATTCAGGACGTTGAGAGGATCTTGGATTATTCCCTCTGCCAAGAAACCTGG
AGCAAAATCAGAGCGGGTCTTCCAATCTCTCCAGTGGATCTCAGCTATCTT
GCTCCTAAAAACCCAGGAACCGGTCCTGCTTTCACCATAATCAATGGTACC
CTAAAATACTTTGAGACCAGATACATCAGAGTCGATATTGCTGCTCCAATC
CTCTCAAGAATGGTCGGAATGATCAGTGGAACTACCACAGAAAGGGAACTG
TGGGATGACTGGGCACCATATGAAGACGTGGAAATTGGACCCAATGGAGTT
CTGAGGACCAGTTCAGGATATAAGTTTCCTTTATACATGATTGGACATGGT
ATGTTGGACTCCGATCTTCATCTTAGCTCAAAGGCTCAGGTGTTCGAACAT
CCTCACATTCAAGACGCTGCTTCGCAACTTCCTGATGATGAGAGTTTATTT
TTTGGTGATACTGGGCTATCCAAAAATCCAATCGAGCTTGTAGAAGGTTGG
TTCAGTAGTTGGAAAAGCTCTATTGCCTCTTTTTTCTTTATCATAGGGTTA
ATCATTGGACTATTCTTGGTTCTCCGAGTTGGTATCCATCTTTGCATTAAA
TTAAAGCACACCAAGAAAAGACAGATTTATACAGACATAGAGATGAGAATT
C

A 4-vector system, which includes a 3-vector lentiviral packaging system, has also been designed and produced using the methods and materials described herein. A schematic of the 4-vector system is shown in FIG. 2. Briefly, and with reference to FIG. 2, the top-most vector is a helper plasmid, which, in this case, does not include Rev. The vector second from the top is a separate Rev plasmid. The vector second from the bottom is the envelope plasmid. The bottom-most vector is the therapeutic vector as described herein.

Referring to FIG. 2, the Helper plasmid includes a CAG enhancer (SEQ ID NO: 18); a CAG promoter (SEQ ID NO: 19); a chicken beta actin intron (SEQ ID NO: 20); a HIV gag (SEQ ID NO: 21); a HIV Pol (SEQ ID NO: 22); a HIV Int (SEQ ID NO: 23); a HIV RRE (SEQ ID NO: 24); and a rabbit beta globin poly A (SEQ ID NO: 26).

The Rev plasmid includes a RSV promoter (SEQ ID NO: 80); a HIV Rev (SEQ ID NO: 25); and a rabbit beta globin poly A (SEQ ID NO: 26).

The Envelope plasmid includes a CMV promoter (SEQ ID NO: 27); a beta globin intron (SEQ ID NO: 28); a VSV-G (SEQ ID NO: 29); and a rabbit beta globin poly A (SEQ ID NO: 30).

Synthesis of a 4-Vector System, which Includes a 3-Vector Lentiviral Packaging System Consisting of Helper, Rev, and Envelope Plasmids.

### Materials and Methods:

Construction of the Helper Plasmid without Rev:

The Helper plasmid without Rev was constructed by inserting a DNA fragment containing the RRE and rabbit beta globin poly A sequence. This sequence was synthesized by MWG Operon with flanking XbaI and XmaI restriction sites. The RRE/rabbit poly A beta globin sequence was then inserted into the Helper plasmid at the XbaI and XmaI restriction sites. The DNA sequence is as follows:

(SEQ ID NO: 34)
TCTAGAAGGAGCTTTGTTCCTTGGGTTCTTGGGAGCAGCAGGAAGCACTAT
GGGCGCAGCGTCAATGACGCTGACGGTACAGGCCAGACAATTATTGTCTGG
TATAGTGCAGCAGCAGAACAATTTGCTGAGGGCTATTGAGGCGCAACAGCA
TCTGTTGCAACTCACAGTCTGGGGCATCAAGCAGCTCCAGGCAAGAATCCT
GGCTGTGGAAAGATACCTAAAGGATCAACAGCTCCTAGATCTTTTTCCCTC
TGCCAAAAATTATGGGGACATCATGAAGCCCCTTGAGCATCTGACTTCTGG
CTAATAAAGGAAATTTATTTTCATTGCAATAGTGTGTTGGAATTTTTTGTG
TCTCTCACTCGGAAGGACATATGGGAGGGCAAATCATTTAAAACATCAGAA
TGAGTATTTGGTTTAGAGTTTGGCAACATATGCCATATGCTGGCTGCCATG
AACAAAGGTGGCTATAAAGAGGTCATCAGTATATGAAACAGCCCCCTGCTG
TCCATTCCTTATTCCATAGAAAAGCCTTGACTTGAGGTTAGATTTTTTTTA
TATTTTGTTTTGTGTTATTTTTTTCTTTAACATCCCTAAAATTTTCCTTAC
ATGTTTTACTAGCCAGATTTTTCCTCCTCTCCTGACTACTCCCAGTCATAG
CTGTCCCTCTTCTCTTATGAAGATCCCTCGACCTGCAGCCCAAGCTTGGCG
TAATCATGGTCATAGCTGTTTCCTGTGTGAAATTGTTATCCGCTCACAATT
CCACACAACATACGAGCCGGAAGCATAAAGTGTAAAGCCTGGGGTGCCTAA
TGAGTGAGCTAACTCACATTAATTGCGTTGCGCTCACTGCCCGCTTTCCAG
TCGGGAAACCTGTCGTGCCAGCGGATCCGCATCTCAATTAGTCAGCAACCA
TAGTCCCGCCCCTAACTCCGCCCATCCCGCCCCTAACTCCGCCCAGTTCCG
CCCATTCTCCGCCCCATGGCTGACTAATTTTTTTTATTTATGCAGAGGCCG
AGGCCGCCTCGGCCTCTGAGCTATTCCAGAAGTAGTGAGGAGGCTTTTTTG
GAGGCCTAGGCTTTTGCAAAAAGCTAACTTGTTTATTGCAGCTTATAATGG
TTACAAATAAAGCAATAGCATCACAAATTTCACAAATAAAGCATTTTTTTC
ACTGCATTCTAGTTGTGGTTTGTCCAAACTCATCAATGTATCTTATCACCC
GGG

### Construction of the Rev Plasmid

The RSV promoter and HIV Rev sequences were synthesized as a single DNA fragment by MWG Operon with flanking MfeI and XbaI restriction sites. The DNA fragment was then inserted into the pCDNA3.1 plasmid (Invitrogen) at the MfeI and XbaI restriction sites in which the CMV promoter is replaced with the RSV promoter. The DNA sequence was as follows:

(SEQ ID NO: 36)
CAATTGCGATGTACGGGCCAGATATACGCGTATCTGAGGGGACTAGGGT
GTGTTTAGGCGAAAAGCGGGGCTTCGGTTGTACGCGGTTAGGAGTCCCC
TCAGGATATAGTAGTTTCGCTTTTGCATAGGGAGGGGGAAATGTAGTCT
TATGCAATACACTTGTAGTCTTGCAACATGGTAACGATGAGTTAGCAAC
ATGCCTTACAAGGAGAGAAAAAGCACCGTGCATGCCGATTGGTGGAAGT
AAGGTGGTACGATCGTGCCTTATTAGGAAGGCAACAGACAGGTCTGACA
TGGATTGGACGAACCACTGAATTCCGCATTGCAGAGATAATTGTATTTA
AGTGCCTAGCTCGATACAATAAACGCCATTTGACCATTCACCACATTGG
TGTGCACCTCCAAGCTCGAGCTCGTTTAGTGAACCGTCAGATCGCCTGG
AGACGCCATCCACGCTGTTTTGACCTCCATAGAAGACACCGGGACCGAT
CCAGCCTCCCCTCGAAGCTAGCGATTAGGCATCTCCTATGGCAGGAAGA
AGCGGAGACAGCGACGAAGAACTCCTCAAGGCAGTCAGACTCATCAAGT
TTCTCTATCAAAGCAACCCACCTCCCAATCCCGAGGGGACCCGACAGGC
CCGAAGGAATAGAAGAAGAAGGTGGAGAGAGAGACAGAGACAGATCCAT
TCGATTAGTGAACGGATCCTTAGCACTTATCTGGGACGATCTGCGGAGC
CTGTGCCTCTTCAGCTACCACCGCTTGAGAGACTTACTCTTGATTGTAA
CGAGGATTGTGGAACTTCTGGGACGCAGGGGGTGGGAAGCCCTCAAATA
TTGGTGGAATCTCCTACAATATTGGAGTCAGGAGCTAAAGAATAGTCTA
GA.

The plasmids used in the packaging systems can be modified with similar elements, and the intron sequences can potentially be removed without loss of vector function. For example, the following elements can replace similar elements in the packaging system:

Promoters: Elongation Factor-1 (EF-1) (SEQ ID NO: 37), phosphoglycerate kinase (PGK) (SEQ ID NO: 38), and ubiquitin C (UbC) (SEQ ID NO: 39) can replace the CMV (SEQ ID NO: 27) or CAG promoter (SEQ ID NO: 19). These sequences can also be further varied by addition, substitution, deletion or mutation.

Poly A sequences: SV40 poly A (SEQ ID NO: 40) and bGH poly A (SEQ ID NO: 41) can replace the rabbit beta globin poly A (SEQ ID NO: 26). These sequences can also be further varied by addition, substitution, deletion or mutation.

HIV Gag, Pol, and Integrase sequences: The HIV sequences in the Helper plasmid can be constructed from different HIV strains or clades. For example, HIV Gag (SEQ ID NO: 21); HIV Pol (SEQ ID NO: 22); and HIV Int (SEQ ID NO: 23) from the Bal strain can be interchanged with the gag, pol, and int sequences contained in the helper/helper plus Rev plasmids as outlined herein. These sequences can also be further varied by addition, substitution, deletion or mutation.

Envelope: The VSV-G glycoprotein can be substituted with membrane glycoproteins from feline endogenous virus (RD114) (SEQ ID NO: 42), gibbon ape leukemia virus (GALV) (SEQ ID NO: 43), Rabies (FUG) (SEQ ID NO: 44), lymphocytic choriomeningitis virus (LCMV) (SEQ ID NO: 45), influenza A fowl plague virus (FPV) (SEQ ID NO: 46), Ross River alphavirus (RRV) (SEQ ID NO: 47), murine leukemia virus 10A1 (MLV) (SEQ ID NO: 81), or Ebola virus (EboV) (SEQ ID NO: 48). Sequences for these envelopes are identified in the sequence portion herein. Further, these sequences can also be further varied by addition, substitution, deletion or mutation.

In summary, the 3-vector versus 4-vector systems can be compared and contrasted as follows. The 3-vector lentiviral vector system contains: 1. Helper plasmid: HIV Gag, Pol, Integrase, and Rev/Tat; 2. Envelope plasmid: VSV-G/FUG envelope; and 3. Therapeutic vector: RSV 5′LTR, Psi Packaging Signal, Gag fragment, RRE, Env fragment, cPPT, WPRE, and 3′8 LTR. The 4-vector lentiviral vector system contains: 1. Helper plasmid: HIV Gag, Pol, and Integrase; 2. Rev plasmid: Rev; 3. Envelope plasmid: VSV-G/FUG envelope; and 4. Therapeutic vector: RSV 5′LTR, Psi Packaging Signal, Gag fragment, RRE, Env fragment, cPPT, WPRE, and 3′delta LTR. Sequences corresponding with the above elements are identified in the sequence listings portion herein.

### Example 2

### Therapeutic Vectors

Exemplary therapeutic vectors have been designed and developed as shown, for example, in FIGS. 3A-3C.

Referring first to FIG. 3A, from left to right, the key genetic elements are as follows: hybrid 5′ long terminal repeat (RSV/LTR), Psi sequence (RNA packaging site), RRE (Rev-response element), cPPT (polypurine tract), H1 promoter, an FDPS shRNA sequence including the FDPS shRNA sequences detailed herein, Woodchuck Post-Transcriptional Regulatory Element (WPRE), and LTR with a deletion in the U3 region.

Referring next to FIG. 3B, from left to right, the key genetic elements are as follows: hybrid 5′ long terminal repeat (RSV/LTR), Psi sequence (RNA packaging site), RRE (Rev-response element), cPPT (polypurine tract), EF-1 alpha (EF-1 alpha promoter of gene transcription), a FDPS miR (miRNA) including the FDPS miRNA sequences detailed herein, Woodchuck Post-Transcriptional Regulatory Element (WPRE), and LTR with a deletion in the U3 region.

To produce the vectors outlined generally in FIGS. 3A-3B, the following methods and materials were employed.

Inhibitory RNA Design: The sequence of Homo sapiens Farnesyl diphosphate synthase (FDPS) (NM_002004.3) mRNA was used to search for potential siRNA or shRNA candidates to knockdown FDPS levels in human cells. Potential RNA interference sequences were chosen from candidates selected by siRNA or shRNA design programs such as from GPP Web Portal hosted by the Broad Institute (portals.broadinstitute.org/gpp/public/) or the BLOCK-iT RNAi Designer from Thermo Scientific (rnaidesigner.thermofisher.com/rnaiexpress/). Individual selected shRNA sequences were inserted into a lentiviral vector immediately 3 prime to a RNA polymerase III promoter H1 (SEQ ID NO: 15) to regulate shRNA expression. These lentivirus shRNA constructs were used to transduce cells and measure the change in specific mRNA levels. The shRNA most potent for reducing mRNA levels were embedded individually within a microRNA backbone to allow for expression by either the EF-1 alpha or CMV RNA polymerase II promoters. The microRNA backbone was selected from mirbase.org. RNA sequences were also synthesized as synthetic siRNA oligonucleotides and introduced directly into cells without using a lentiviral vector.

Vector Construction: For FDPS shRNA, oligonucleotide sequences containing BamHI and EcoRI restriction sites were synthesized by Eurofins MWG Operon. Overlapping sense and antisense oligonucleotide sequences were mixed and annealed during cooling from 70 degrees Celsius to room temperature. The lentiviral vector was digested with the restriction enzymes BamHI and EcoRI for one hour at 37 degrees Celsius. The digested lentiviral vector was purified by agarose gel electrophoresis and extracted from the gel using a DNA gel extraction kit from Thermo Scientific. The DNA concentrations were determined and vector to oligo (3:1 ratio) were mixed, allowed to anneal, and ligated. The ligation reaction was performed with T4 DNA ligase for 30 minutes at room temperature. 2.5 microliters of the ligation mix were added to 25 microliters of STBL3 competent bacterial cells. Transformation was achieved after heat-shock at 42 degrees Celsius. Bacterial cells were spread on agar plates containing ampicillin and drug-resistant colonies (indicating the presence of ampicillin-resistance plasmids) were recovered and expanded in LB broth. To check for insertion of the oligo sequences, plasmid DNA was extracted from harvested bacteria cultures with the Thermo Scientific DNA mini prep kit. Insertion of shRNA sequences in the lentiviral vector was verified by DNA sequencing using a specific primer for the promoter used to regulate shRNA expression. Using the following target sequences, exemplary shRNA sequences were determined to knock-down FDPS:

GTCCTGGAGTACAATGCCATT (FDPS target sequence; SEQ
ID NO: 49);
GTCCTGGAGTACAATGCCATTCTCGAGAATGGCATTGTACTCCAGGACTT
TTT (FDPS shRNA sequence #1; SEQ ID NO: 1);
GCAGGATTTCGTTCAGCACTT (FDPS target sequence #2;
SEQ ID NO: 50);
GCAGGATTTCGTTCAGCACTTCTCGAGAAGTGCTGAACGAAATCCTGCTT
TTT (FDPS shRNA sequence #2; SEQ ID NO: 2);
GCCATGTACATGGCAGGAATT (FDPS target sequence #3;
SEQ ID NO: 51);
GCCATGTACATGGCAGGAATTCTCGAGAATTCCTGCCATGTACATGGCTT
TTT (FDPS shRNA sequence #3; SEQ ID NO: 3);
GCAGAAGGAGGCTGAGAAAGT (FDPS target sequence #4;
SEQ ID NO: 52);
and
GCAGAAGGAGGCTGAGAAAGTCTCGAGACTTTCTCAGCCTCCTTCTGCTT
TTT (FDPS shRNA sequence #4; SEQ ID NO: 4).

shRNA sequences were then assembled into a synthetic microRNA (miR) under control of the EF-1 alpha promoter. Briefly, a miR hairpin sequences, such as miR30, miR21, or miR185 as detailed below, was obtained from mirbase.org. The 19-22mer shRNA target sequence was used to construct the synthetic miR sequence. The miR sequence was arranged as an anti-sense-target-sequence-hairpin loop sequence (specific for each microRNA)-sense target sequence.

The following miR sequences were developed:

GTGAAGCCACAGATGGCAGAAGGAGGCTGAGAAAGTGCTGCCTACTGCCT
CGGACTTCAAGGGGCT (miR30 FDPS sequence #1; SEQ ID
NO: 53)
AAGGTATATTGCTGTTGACAGTGAGCGACACTTTCTCAGCCTCCTTCTGC
GTGAAGCCACAGATGGCAGAAGGGCTGAGAAAGTGCTGCCTACTGCCTCG
GACTTCAAGGGGCT (miR30 FDPS sequence #2; SEQ ID
NO: 54)
TGCTGTTGACAGTGAGCGACTTTCTCAGCCTCCTTCTGCGTGAAGCCACA
GATGGCAGAAGGAGGCTGAGAAAGTTGCCTACTGCCTCGGA (miR30
FDPS sequence #3; SEQ ID NO: 55)
CCTGGAGGCTTGCTGAAGGCTGTATGCTGACTTTCTCAGCCTCCTTCTGC
TTTTGGCCACTGACTGAGCAGAAGGGCTGAGAAAGTCAGGACACAAGGCC
TGTTACTAGCACTCA (miR155 FDPS sequence #1; SEQ
ID NO: 56)
CATCTCCATGGCTGTACCACCTTGTCGGGACTTTCTCAGCCTCCTTCTGC
CTGTTGAATCTCATGGCAGAAGGAGGCGAGAAAGTCTGACATTTTGGTAT
CTTTCATCTGACCA (miR21 FDPS sequence #1; SEQ ID
NO: 57)
GGGCCTGGCTCGAGCAGGGGGCGAGGGATACTTTCTCAGCCTCCTTCTGC
TGGTCCCCTCCCCGCAGAAGGAGGCTGAGAAAGTCCTTCCCTCCCAATGA
CCGCGTCTTCGTCG (miR185 FDPS sequence #1; SEQ ID
NO: 58)

Combination vectors, as shown generally in FIG. 3C are also capable of being produced based on the development of the single-target vectors outlined above. An exemplary therapeutic combination vectors is shown in FIG. 3C, and includes from left to right: hybrid 5′ long terminal repeat (RSV/LTR), Psi sequence (RNA packaging site), RRE (Rev-response element), cPPT (polypurine tract), EF-1alpha (EF-1alpha promoter of gene transcription), miR30-FDPS, miR155-CD47, miR21-cMyc, Woodchuck Post-Transcriptional Regulatory Element (WPRE), and LTR with a deletion in the U3 region. The therapeutic vector detailed in FIG. 3C can be produced using the materials and methods described using the following target sequences:

miR30 FDPS sequence #1:
(SEQ ID NO: 53)
AAGGTATATTGCTGTTGACAGTGAGCGACACTTTCTCAGCCTCCTTCTGCG
TGAAGCCACAGATGGCAGAAGGAGGCTGAGAAAGTGCTGCCTACTGCCTCG
GACTTCAAGGGGCT
miR155 CD47 target sequence #1:
(SEQ ID NO: 82)
CCTGGAGGCTTGCTGAAGGCTGTATGCTGTTATCCATCTTCAAAGAGGCAG
TTTTGGCCACTGACTGACTGCCTCTTAAGATGGATAACAGGACACAAGGCC
TGTTACTAGCACTCA
miR21 cMyc sequence:
(SEQ ID NO: 83)
CATCTCCATGGCTGTACCACCTTGTCGGGTGTTCGCCTCTTGACATTCTCC
TGTTGAATCTCATGGAGAATGTCAAGGGCGAACACTGACATTTTGGTATCT
TTCATCTGACCA

### Example 3

### Materials and Methods for FDPS

Inhibitory RNA Design: The sequence of Homo sapiens farnesyl diphosphate synthase (FDPS), transcript variant 1, mRNA (NM_002004.3) was used to search for potential siRNA or shRNA candidates to knockdown FDPS levels in human cells. Potential RNA interference sequences were chosen from candidates selected by siRNA or shRNA design programs such as from the Broad Institute or the BLOCK-iT™ RNAi Designer from Thermo Scientific. A shRNA sequence may be inserted into a lentiviral vector after a RNA polymerase III promoter such as H1, U6, or 7SK to regulate shRNA expression. The RNA sequence may also be embedded within a microRNA backbone to allow for expression by a RNA polymerase II promoter such as CMV or EF-1 alpha. The RNA sequence may also be synthesized as a siRNA oligonucleotide and utilized independently of a lentiviral vector.

Vector Construction: For FDPS shRNA, oligonucleotide sequences containing BamHI and EcoRI restriction sites were synthesized by MWG operon. Oligonucleotide sequences were annealed by incubation at 70 degrees Celsius and cooling to room temperature. Annealed oligonucleotides were digested with the restriction enzymes BamHI and EcoRI for one hour at 37 degrees Celsius and then the enzymes were heat-inactivated at 70 degrees Celsius for 20 minutes. In parallel, the lentiviral vector was digested with the restriction enzymes BamHI and EcoRI for one hour at 37 degrees Celsius. The digested lentiviral vector was purified by agarose gel electrophoresis and extracted from the gel using a DNA gel extraction kit from Invitrogen. The DNA concentration was determined and the vector to oligo sequence was ligated in the ratio 3:1 insert to vector. The ligation reaction was carried out with T4 DNA ligase for 30 minutes at room temperature. 2.5 microliters of the ligation mix was added to 25 microliters of STBL3 competent bacterial cells. Transformation was carried out by heat-shock at 42 degrees Celsius. Bacterial cells were streaked onto agar plates containing ampicillin and then colonies were expanded in LB broth. To check for insertion of the oligo sequences, plasmid DNA was extracted from harvested bacteria cultures with the Invitrogen DNA mini prep kit. Insertion of the shRNA sequence in the lentiviral vector was verified by DNA sequencing using a specific primer for which every promoter is used to regulate shRNA expression. The lentiviral vectors containing a correct FDPS sequence were then used to package lentiviral particles to test for their ability to knockdown FDPS. Mammalian cells were transduced with lentiviral particles either in the presence or absence of polybrene. Cells were collected after 2-4 days and protein and RNA was analyzed for FDPS expression.

Functional Assay for mRNA Reduction: The effect of different FDPS short homology RNA (shRNA) targeting sequences on FDPS expression was determined by measuring mRNA expression. HepG2 hepatocellular carcinoma cells were transduced with a lentiviral vector containing FDPS shRNA sequences. After 48 hours, cells were lysed and RNA was extracted using the RNeasy mini kit from Qiagen. cDNA was then synthesized from RNA using SuperScript VILO from Invitrogen. The samples were then analyzed by quantitative RT-PCR using an Applied Biosystems StepOne PCR machine. FDPS expression was detected with SYBR Green from Invitrogen using the forward primer (5′-AGGAATTGATGGCGAGAAGG-3′) (SEQ ID NO: 59) and reverse primer (5′-CCCAAAGAGGTCAAGGTAATCA-3′) (SEQ ID NO: 60) with standard conditions for polymerase chain reaction analysis. The samples were normalized to the mRNA for beta-actin gene expression using the forward primer (5′-AGCGCGGCTACAGCTTCA-3′) (SEQ ID NO: 61) and reverse primer (5′-GGCGACGTAGCACAGCTTCT-3′) (SEQ ID NO: 62) with standard conditions for polymerase chain reaction analysis. The relative expression of FDPS was determined by its Ct value normalized to the level of actin for each sample.

Functional Assay for tumor cells modified by LV-FDPS and used to activate cytokine production in human gamma delta T cells: The LV-FDPS vector was also used to treat tumor cells that were then exposed to primary human gamma delta T cells from healthy donors. Combined treatment of tumor cell line with both aminobisphosphonate and vector that suppresses farnesyl pyrophosphate synthase (FDPS) has a synergistic effect on gamma delta T cell production of TNF-alpha. THP1 monocytoid tumor cell line (A) or HepG2 monocytoid tumor cell line (B) were treated with lentiviral control vectors (LV-Control), lentiviral vectors expressing shRNA to down regulate FDPS (LV-FDPS), zoledronic acid (Zol), zoledronic acid plus lentiviral control (Zol+LV-Control), or zoledronic acid plus lentiviral vectors expressing shRNA to down regulate FDPS (Zol+LV-FDPS). Treated cells were mixed with gamma delta T cells at 1:1 ratio for 4 hours. TNF-alpha production by gamma delta T cells was detected by intracellular staining and flow cytometry.

Functional Assay for tumor cells modified by LV-FDPS and used to activate tumor cell killing by human gamma delta T cells: Monocytoid tumor cells (THP-1) were transduced with lentivirus vector that suppresses FDPS mRNA, then used to activate tumor cell cytotoxicity in normal human gamma delta T cells. The activated gamma delta T cells were recovered after 4 hours of exposure to transduced THP-1 cells, then used in a cytotoxicity assay to kill unmodified THP-1. When gamma delta T cells were stimulated with a combination of transduced THP-1 cells and 10 micromolar zoledronic acid, >70% killing of THP-1 was observed at a ratio of 4 gamma delta T cells to 1 THP-1 cell.

### Experimental Data for FDPS

The FDPS shRNA sequences depicted in Table 2 were utilized in the experiments described herein. Further, the sequences detailed in Table 2 can be used in the therapeutic vectors detailed herein.

TABLE 2
FDPS shRNA Sequences

| | | SEQ |
|---|---|---|
| | shRNA oligonucleotide (sense | ID |
| Description | sequence-loop-antisense sequence | NO |
| FDPS-1 | GTCCTGGAGTACAATGCCATTCTCGAGAATGG | 1 |
| | CATTGTACTCCAGGACTTTTT | |
| FDPS-2 | GCAGGATTTCGTTCAGCACTTCTCGAGAAGTG | 2 |
| | CTGAACGAAATCCTGCTTTTT | |
| FDPS-3 | GCCATGTACATGGCAGGAATTCTCGAGAATTC | 3 |
| | CTGCCATGTACATGGCTTTTT | |
| FDPS-4 | GCAGAAGGAGGCTGAGAAAGTCTCGAGACTTT | 4 |
| | CTCAGCCTCCTTCTGCTTTTT | |

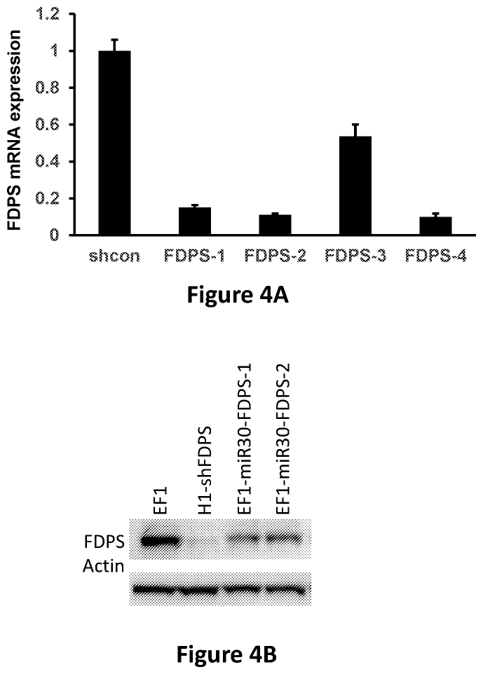

As shown in FIG. 4A, the relative expression level of human FDPS following administration of the four different FDPS shRNA sequences was determined. The most significant inhibition of human FDPS expression was found in the FDPS-2 and FDPS-4 samples (as shown in FIG. 4A, herein).

Further, as shown in FIG. 4B, a lentiviral-based delivery system was used to target FDPS expression. HepG2 human hepatocellular carcinoma cells were infected with lentiviral vectors containing either the H1 promoter and a FDPS shRNA (SEQ ID NO: 4) sequence or the EF-1alpha promoter and the following miR30-based FDPS sequences:

miR30 FDPS sequence #1:
(SEQ ID NO: 53)
AAGGTATATTGCTGTTGACAGTGAGCGACACTTTCTCAGCCTCCTTCTGCG
TGAAGCCACAGATGGCAGAAGGAGGCTGAGAAAGTGCTGCCTACTGCCTCG
GACTTCAAGGGGCT
miR30 FDPS sequence #2:
(SEQ ID NO: 54)
AAGGTATATTGCTGTTGACAGTGAGCGACACTTTCTCAGCCTCCTTCTGCG
TGAAGCCACAGATGGCAGAAGGGCTGAGAAAGTGCTGCCTACTGCCTCGGA
CTTCAAGGGGCT

After 48 hours, cells were lysed and an immunoblot was performed using an anti-FDPS (Thermo Scientific) and an anti-actin (Sigma) antibody for a protein loading control. As shown in FIG. 4B, treatment with the FDPS shRNA significantly decreased FDPS protein expression. Treatment with the miR30-based FDPS sequences decreased FDPS expression.

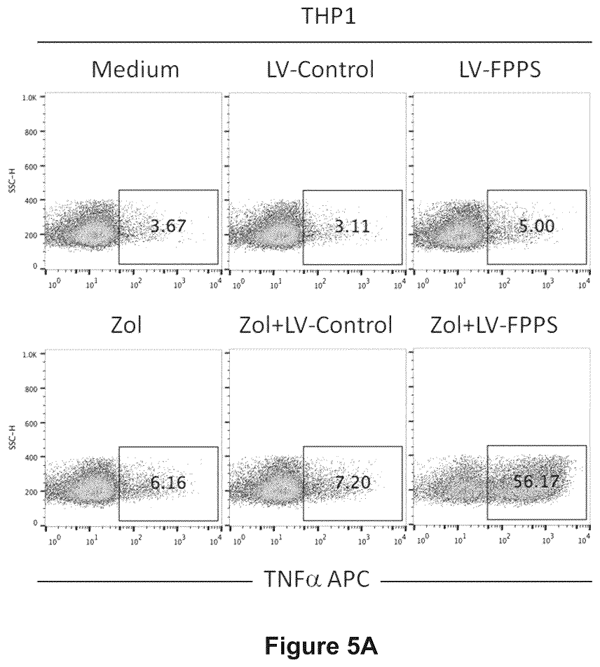

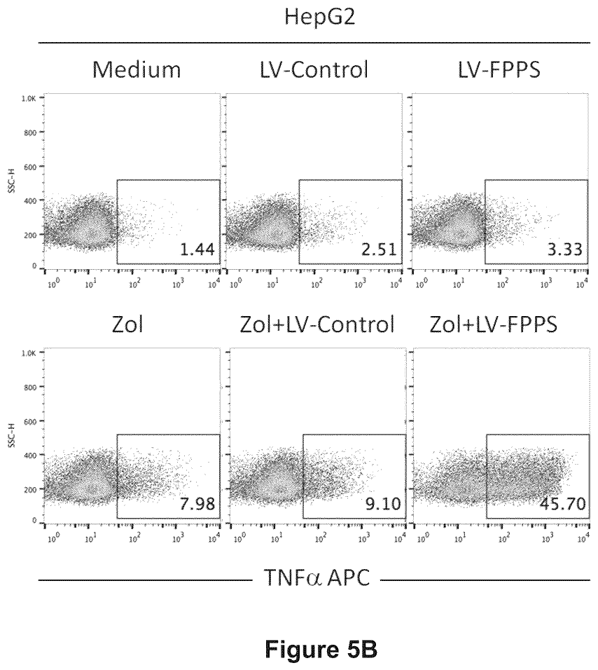

As shown in FIGS. 5A-5B, monocytoid (THP-1) (FIG. 5A) or hepatocellular (HepG2) (FIG. 5B) cancer cells transduced with lentivirus containing shRNA capable of suppressing FDPS mRNA activated cytokine expression in human gamma delta T cells.

This portion of the Example illustrates that knock-down of FDPS in THP1 monocytic leukemia cells by lentiviral (LV)-expressing FDPS shRNA (SEQ ID NO: 4; which is also referred to herein as LV-FDPS shRNA #4) stimulates TNF-α expression in gamma delta T cells, as shown in FIG. 5A.

THP1 cells (1×10⁵ cells) were transduced with LV-control or LV-FDPS shRNA #4 for 3 days. Two days after transduction, cells were treated with or without 1 μM zoledronic acid. After 24 hours, the transduced THP-1 cells were co-cultured with 5×10⁵ PBMC cells and IL-2 in a round bottom 96 well plate for 4 hours. The PBMC cells were pre-stimulated with zoledronic acid and IL-2 for 11 days to expand Vγ9Vδ2 T cells. After staining for Vγ9Vδ2 and TNF-α using fluorophore-conjugated anti TCR-Vδ2 and anti-TNF-α antibody, cells were analyzed via flow cytometry. Live cells were gated, and Vδ2+ and TNF-α+ cells were selected on a dot blot. The activated cytotoxic Vγ9Vδ2 T cells appeared in the upper right quadrant of flow cytograms. Without zoledronic acid, LV-control stimulated 3.11% of TNF-α expressing Vγ9Vδ2 T cells and LV-FDPS shRNA #4 stimulated 5%. With zoledronic acid treatment, LV-control stimulated 7.2% of TNF-α expressing Vγ9Vδ2 T cells and LV-FDPS shRNA #4 stimulated 56.17%.

The same conditions were used with HepG2 cells and the following data was generated. Without zoledronic acid, LV-control stimulated 2.5% of TNF-α expressing Vγ9Vδ2 T cells and LV-FDPS shRNA #4 stimulated 3.33%. With zoledronic acid treatment, LV-control stimulated 9.1% of TNF-α expressing Vγ9Vδ2 T cells and LV-FDPS shRNA #4 stimulated 45.7%.

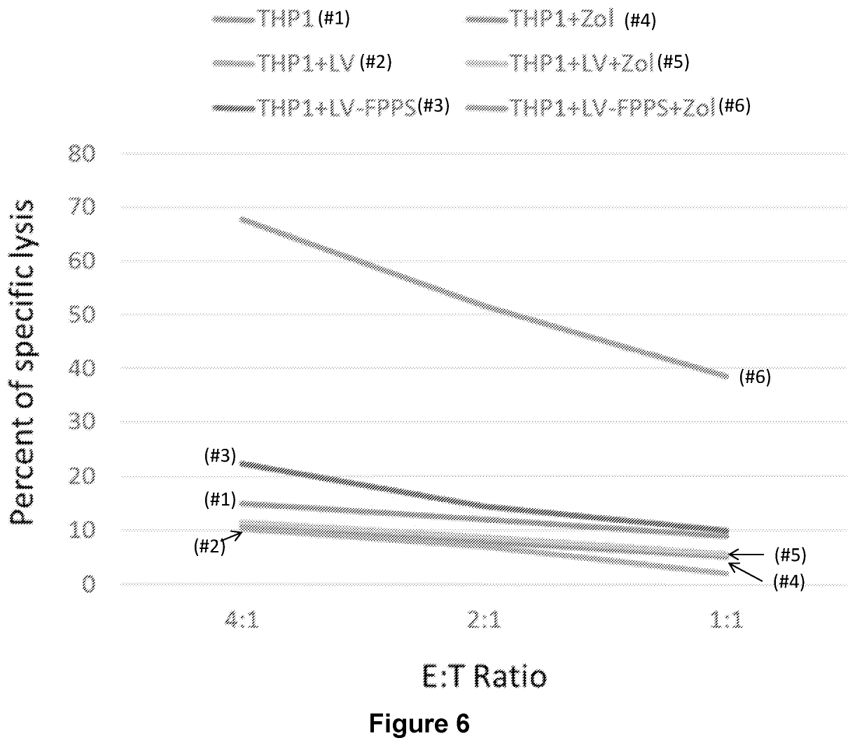

Further as shown in FIG. 6, monocytoid (THP-1) tumor cells transduced with lentivirus capable of suppressing FDPS mRNA activate tumor cell cytotoxicity in normal human gamma delta T cells.

This portion of the Example demonstrates results from mixing treated THP-1 monocytoid tumor cells with cultured human GD T cells, as shown in FIG. 6.

The monocytoid cell line THP-1 was treated with control lentivirus vector (LV), LV suppressing farnesyl diphosphate synthase gene expression (LV-FDPS), zoledronic acid (Zol) or combinations. The legend, as shown in FIG. 6, was: lentiviral control vectors (LV-Control), lentiviral vectors expressing microRNA to down regulate FDPS (LV-FDPS), Zometa (Zol), Zometa plus lentiviral control (Zol+LV-Control), or Zometa plus lentiviral vectors expressing microRNA to down regulate FDPS (Zol+LV-FDPS).

Human GD T cells were cultured from an anonymous donor and added to treated THP-1 cells in 4:1, 2:1 or 1:1 ratios (GD T:THP-1) for 4 hours. Cell killing was measured by a fluorescence assay. When THP-1 cells were treated with a combination of LV-FDPS and Zol, cytotoxic T cell killing by GD T cells was increased greatly compared to either treatment alone. When LV-FDPS treatment alone was compared to Zol treatment alone, the LV-FDPS lead to greater killing but was >3-fold below tumor cell killing after combination treatment. The combined LV-FDPS plus Zol treatment caused nearly 70% tumor cell killing with 4:1 ratio; this was more than 3-fold higher than the second best treatment (LV-FDPS alone).

### Example 4

### Materials and Methods for CD47

Inhibitory RNA Selection: The sequence of Homo sapiens CD47 molecule (CD47) mRNA (NM_001777) was used to search for potential siRNA or shRNA candidates capable of reducing CD47 levels in human cells. Potential RNA interference sequences were chosen from candidates selected by siRNA or shRNA design programs such as from the Broad Institute or the BLOCK-iT™ RNAi Designer from Thermo Scientific. Initially, individual selected shRNA sequences were inserted into lentiviral vectors immediately 3′ to a RNA polymerase III promoter such as H1, U6, or 7SK to regulate shRNA expression. These lentivirus-shRNA constructs were used to transduce cells and measure the change in specific mRNA levels. The shRNA most potent for reducing mRNA levels were embedded individually within a microRNA backbone to allow for expression by either the CMV or EF-1 alpha RNA polymerase II promoters. RNA sequences have also been synthesized as synthetic siRNA oligonucleotides and introduced directly into cells without using a lentiviral vector.

Vector Construction: For CD47 shRNA, oligonucleotide sequences containing BamHI and EcoRI restriction sites were synthesized by Eurofins MWG Operon, LLC. Overlapping sense and antisense oligonucleotide sequences were mixed and annealed during incubation at 70 degrees Celsius before being cooled to room temperature and extending the unpaired ends with DNA polymerase before cooling to room temperature. The extension reaction created double stranded sequences at each end of the oligonucleotide that contain restriction enzyme sites BamHI and EcoRI. The double stranded oligonucleotides were digested with the restriction enzymes BamHI and EcoRI for one hour at 37 degrees Celsius and the enzymes were heat-inactivated at 70 degrees Celsius for 20 minutes. In parallel, the lentiviral vector was digested with the restriction enzymes BamHI and EcoRI for one hour at 37 degrees Celsius. The digested lentiviral vector was purified by agarose gel electrophoresis and extracted from the gel using a DNA gel extraction kit from Invitrogen. The DNA concentrations were determined and vector to oligo (3:1 ratio) were mixed, allowed to anneal, and ligated. The ligation reaction was performed with T4 DNA ligase for 30 minutes at room temperature. 2.5 microliters of the ligation mix were added to 25 microliters of STBL3 competent bacterial cells. Transformation was achieved after heat-shock at 42 degrees Celsius. Bacterial cells were spread on agar plates containing ampicillin and drug-resistant colonies (indicating the presence of ampicillin-resistance plasmids) were recovered, purified and expanded in LB broth. To check for insertion of the oligo sequences, plasmid DNA were extracted from harvested bacteria cultures with the Invitrogen DNA mini prep kit. Insertion of the shRNA sequence in the lentiviral vector was verified by DNA sequencing using a specific primer for the promoter used to regulate shRNA expression.

Functional Assay: The effect of different CD47 shRNA targeting sequences on CD47 expression was determined by measuring mRNA expression. Hep3B hepatocellular carcinoma cells were transduced with a lentiviral vector containing CD47 shRNA sequences. After 48 hours, cells were lysed and RNA was extracted using the RNeasy mini kit from Qiagen. cDNA was then synthesized from RNA using SuperScript VILO from Invitrogen. The samples were then analyzed by quantitative RT-PCR using an Applied Biosystems StepOne PCR machine. CD47 expression was detected with SYBR Green from Invitrogen using the forward primer (5′-CACTGTCGTCATTCCATGCT-3′) (SEQ ID NO: 63) and reverse primer (5′-GCCTCTTGACATTCTCCTC-3′) (SEQ ID NO: 64). The samples were normalized by measuring actin expression using the forward primer (5′-AGCGCGGCTACAGCTTCA-3′) (SEQ ID NO: 61) and reverse primer (5′-AAAGTCAGTGGGGACAGTGG-3′) (SEQ ID NO: 65). The relative expression of CD47 was determined by its Ct value normalized to the level of actin for each sample.

### Experimental Data for CD47

The non-limiting examples of CD47 shRNA target sequences depicted in Table 3 were utilized in the experiments described herein. Further, the sequences detailed in Table 3 can be used in the therapeutic vectors detailed herein.

TABLE 3
CD47 shRNA sequences

| | | SEQ |
|---|---|---|
| | shRNA oligonucleotide (sense | ID |
| Description | sequence-loop-antisense sequence | NO |
| CD47 | GGTGAAACGATCATCGAGCCTCGAGGCTCGAT | 5 |
| sequence 1 | GATCGTTTCACCTTTTT | |
| CD47 | GCTACTGGCCTTGGTTTAACTCGAGTTAAACC | 6 |
| sequence 2 | AAGGCCAGTAGCTTTTT | |
| CD47 | CCTCCTTCGTCATTGCCATCTCGAGATGGCAA | 7 |
| sequence 3 | TGACGAAGGAGGTTTTT | |
| CD47 | GCATGGCCCTCTTCTGATTCTCGAGAATCAGA | 8 |
| sequence 4 | AGAGGGCCATGCTTTTT | |
| CD47 | GGTGAAACGATCATCGAGCTACTCGAGTAGCT | 9 |
| sequence 5 | CGATGATCGTTTCACCTTTTT | |

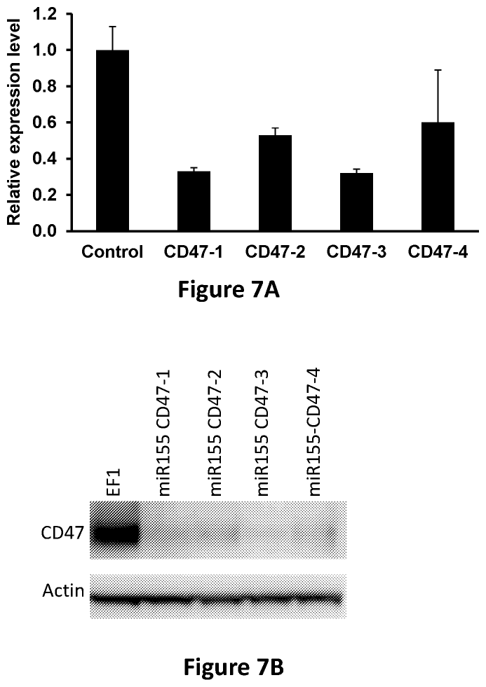

As shown in FIG. 7A, the relative expression level of human CD47 following administration of the four different CD47 shRNA sequences was determined. The most significant inhibition of human CD47 expression was found in the shCD47-1 and shCD47-3 samples (as shown in FIG. 7A, herein).

Further, as shown in FIG. 7B, a lentiviral-based delivery system was used to target CD47 expression. SNU449 human hepatocellular carcinoma cells were infected with lentiviral vectors containing the following miR155-based CD47 sequences:

miR155 CD47 target sequence #1:
(SEQ ID NO: 82)
CCTGGAGGCTTGCTGAAGGCTGTATGCTGTTATCCATCTTCAAAGAGGC
AGTTTTGGCCACTGACTGACTGCCTCTTAAGATGGATAACAGGACACAA
GGCCTGTTACTAGCACTCA
miR155 CD47 target sequence #2:
(SEQ ID NO: 66)
CCTGGAGGCTTGCTGAAGGCTGTATGCTGTTAGCTCGATGATCGTTTCA
CGTTTTGGCCACTGACTGACGTGAAACGCATCGAGCTAACAGGACACAA
GGCCTGTTACTAGCACTCA
miR155 CD47 target sequence #3:
(SEQ ID NO: 67)
CCTGGAGGCTTGCTGAAGGCTGTATGCTGAAGAATGGCTCCAACAATGA
CGTTTTGGCCACTGACTGACGTCATTGTGAGCCATTCTTCAGGACACAA
GGCCTGTTACTAGCACTCA
miR155 CD47 target sequence #4:
(SEQ ID NO: 68)
CCTGGAGGCTTGCTGAAGGCTGTATGCTGTATACACGCCGCAATACAGA
GGTTTTGGCCACTGACTGACCTCTGTATCGGCGTGTATACAGGACACAA
GGCCTGTTACTAGCACTCA

As shown in FIG. 7B, treatment with the CD47 shRNA significantly decreased FDPS protein expression. Treatment with the miR155-based CD47 sequences significant decreased CD47 expression.

### Example 5

### Materials and Methods for cMyc

Inhibitory RNA Design: The mRNA sequence of Homo sapiens v-myc avian myelocytomatosis viral oncogene homolog (MYC) (NM_002467.4) was used to screen for potential shRNA candidates to knock-down MYC expression in hepatocellular cell lines. We obtained five MYC shRNA sequences which can reduce MYC expression. Potential RNA interference sequences were chosen from candidates selected by siRNA or shRNA design programs such as from the Broad Institute or the BLOCK-iT™ RNAi Designer from Thermo Scientific. A shRNA sequence may be inserted into a lentiviral vector after a RNA polymerase III promoter such as H1, U6, or 7SK to regulate shRNA expression. The RNA sequence may also be embedded within a microRNA backbone to allow for expression by a RNA polymerase II promoter such as CMV or EF-1 alpha. The RNA sequence may also be synthesized as a siRNA oligonucleotide and utilized independently of a lentiviral vector.

Vector Construction: For cMyc shRNA, oligonucleotide sequences containing BamHI and EcoRI restriction sites were synthesized by MWG operon. Oligonucleotide sequences were annealed by incubation at 70 degrees Celsius and cooling to room temperature. Annealed oligonucleotides were digested with the restriction enzymes BamHI and EcoRI for one hour at 37 degrees Celsius and then the enzymes were heat-inactivated at 70 degrees Celsius for 20 minutes. In parallel, the lentiviral vector was digested with the restriction enzymes BamHI and EcoRI for one hour at 37 degrees Celsius. The digested lentiviral vector was purified by agarose gel electrophoresis and extracted from the gel using a DNA gel extraction kit from Invitrogen. The DNA concentration was determined and the vector to oligo sequence was ligated in the ratio 3:1 insert to vector. The ligation reaction was carried out with T4 DNA ligase for 30 minutes at room temperature. 2.5 microliters of the ligation mix was added to 25 microliters of STBL3 competent bacterial cells. Transformation was carried out by heat-shock at 42 degrees Celsius. Bacterial cells were streaked onto agar plates containing ampicillin and then colonies were expanded in LB broth. To check for insertion of the oligo sequences, Plasmid DNA was extracted from harvested bacteria cultures with the Invitrogen DNA mini prep kit. Insertion of the shRNA sequence in the lentiviral vector was verified by DNA sequencing using a specific primer for which ever promoter is used to regulate shRNA expression. The lentiviral vectors containing a correct cMyc sequence were then used to package lentiviral particles to test for their ability to knockdown FDPS. Mammalian cells were transduced with lentiviral particles either in the presence or absence of polybrene. Cells were collected after 2-4 days and protein and RNA was analyzed for cMyc expression.

Functional Assay: The effect of different cMyc shRNA targeting sequences on cMyc expression was determined by measuring mRNA expression. HepG2 hepatocellular carcinoma cells were transduced with a lentiviral vector containing cMyc shRNA sequences. After 48 hours, cells were lysed and RNA was extracted using the RNeasy mini kit from Qiagen. cDNA was then synthesized from RNA using SuperScript VILO from Invitrogen. The samples were then analyzed by quantitative PCR using an Applied Biosystems StepOne PCR machine. cMyc expression was detected with SYBR Green from Invitrogen using the forward primer (5′-GGACTATCCTGCTGCCAA-3′) (SEQ ID NO: 69) and reverse primer (5′-GCCTCTTGACATTCTCCTC-3′) (SEQ ID NO: 64). The samples were normalized by measuring actin expression using the forward primer (5′-AGCGCGGCTACAGCTTCA-3′) (SEQ ID NO: 61) and reverse primer (5′-GGCGACGTAGCACAGCTTCT-3′) (SEQ ID NO: 62). The relative expression of cMyc was determined by its Ct value normalized to the level of actin for each sample.

Experimental Data for cMyc

The non-limiting examples of cMyc shRNA sequences depicted in Table 4 below were utilized in the experiments described herein.

TABLE 4
cMyc shRNA sequences

| | | SEQ |
|---|---|---|
| | shRNA oligonucleotide (sense | ID |
| Description | sequence-loop-antisense sequence | NO |
| cMyc shRNA | GCTTCACCAACAGGAACTATGCTCGAGCATAG | 10 |
| Sequence 1 | TTCCTGTTGGTGAAGCTTTT | |
| cMyc shRNA | GCGAACACACAACGTCTTGGACTCGAGTCCAA | 11 |
| Sequence 2 | GACGTTGTGTGTTCGCTTTT | |
| cMyc shRNA | GACATGGTGAACCAGAGTTTCCTCGAGGAAAC | 12 |
| Sequence 3 | TCTGGTTCACCATGTCTTTTT | |
| cMyc shRNA | GAGAATGTCAAGAGGCGAACACTCGAGTGTTC | 13 |
| Sequence 4 | GCCTCTTGACATTCTCTTTTT | |
| cMyc shRNA | GCTCATTTCTGAAGAGGACTtCTCGAGAAGTC | 14 |
| Sequence 5 | CTCTTCAGAAATGAGCTTTTT | |

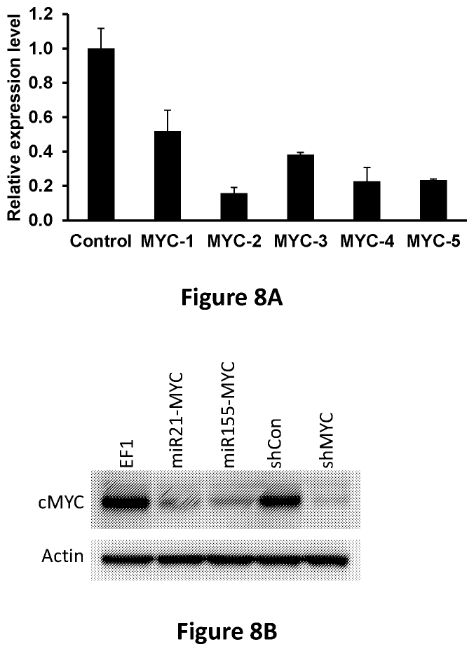

As shown in FIG. 8A, the relative expression level of human cMyc following administration of the five different cMyc shRNA sequences was determined. The most significant inhibition of human cMyc expression was found in the myc-2 sample (as shown in FIG. 8A, herein).

Further, as shown in FIG. 8B, SNU449 human hepatocellular carcinoma cells were infected with lentiviral vectors containing either the following miR-based cMYC sequences or a cMyc shRNA:

miR155 cMyc sequence:
(SEQ ID NO: 70)
CCTGGAGGCTTGCTGAAGGCTGTATGCTGTGTTCGCCTCTTGACATTCTCT
TTTGGCCACTGACTGAGAGAATGTAGAGGCGAACACAGGACACAAGGCCTG
TTACTAGCACTCA
miR21 cMyc sequence:
(SEQ ID NO: 83)
CATCTCCATGGCTGTACCACCTTGTCGGGTGTTCGCCTCTTGACATTCTCC
TGTTGAATCTCATGGAGAATGTCAAGGGCGAACACTGACATTTTGGTATCT
TTCATCTGACCA

The above two cMyc sequences were generated using the below target sequence:

cMyc target sequence:
(SEQ ID NO: 71)
GAGAATGTCAAGAGGCGAACA
cMyc shRNA sequence:
(SEQ ID NO: 13)
GAGAATGTCAAGAGGCGAACACTCGAGTGTTCGCCTCTTGACATTCTCTTT
TT

After 48 hours, cells were lysed and an immunoblot was performed using an anti-cMyc (Santa Cruz) and an anti-actin (Sigma) antibody for a protein loading control. As shown in FIG. 8B, treatment with the cMyc shRNA significantly decreased cMyc protein expression. Treatment with the miR-based cMyc sequences also decreased cMyc expression.

### Example 6

### In Vivo Treatment with FDPS-SHRNA and Zoledronic Acid

Protocol overview for co-administration of LV-shRNA-FDPS (farnesyl diphosphate synthase) with or without zoledronic acid in mice implanted with human prostate cancer cell line PC3. Tumor cells were cultured in vitro, then transduced with lentivirus vector control with a scrambled sequence (nonfunctional) shRNA insert and an expression cassette for firefly luciferase, or LV-FDPS with a shRNA capable of reducing expression of FDPS mRNA and an expression cassette for firefly luciferase. The transduced tumor cells were implanted on the flank of immune deficient mice by subcutaneous injection. Once tumors reached approximately 200 mm³ volume, all mice receive a single dose of zoledronic acid (100 micrograms per kilogram body weight, which is similar to a standard human dose) in saline. 7 days after zoledronic acid injection, an imaging study was repeated to measure volume and photon intensity of individual tumors.

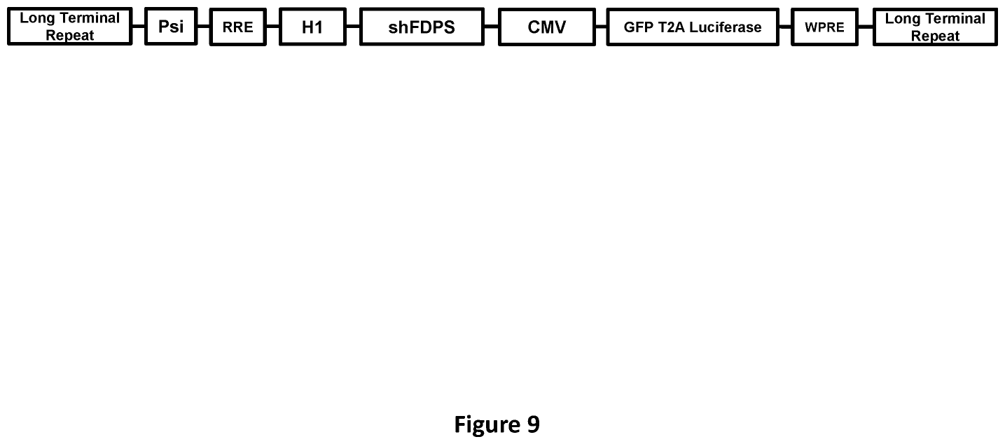

The LV-FDPS vector designed, developed, and utilized in this Example is shown diagrammatically in FIG. 9. The LV-FDPS vector was developed using the methods and materials described herein. The following sequences were used and, as described below, a CMV GFP T2A luciferase sequence was generated and introduced into the therapeutic vector.

CMV promoter sequence:
(SEQ ID NO: 72)
ATTATGCCCAGTACATGACCTTATGGGACTTTCCTACTTGGCAGTACATCTACGTATTAG
TCATCGCTATTACCATGGTGATGCGGTTTTGGCAGTACATCAATGGGCGTGGATAGCGGT
TTGACTCACGGGGATTTCCAAGTCTCCACCCCATTGACGTCAATGGGAGTTTGTTTTGGC
ACCAAAATCAACGGGACTTTCCAAAATGTCGTAACAACTCCGCCCCATTGACGCAAATGG
GCGGTAGGCGTGTACGGTGGGAGGTTTATATAAGCAGAGCTCGTTTAGTGAACCGTCAGA
TCGCCTGGAGACGCCATCCACGCTGTTTT
GFP T2A Luciferase sequence:
(SEQ ID NO: 73)
ATGCCCGCCATGAAGATCGAGTGCCGCATCACCGGCACCCTGAACGGCGTGGAGTTCGAG
CTGGTGGGCGGCGGAGAGGGCACCCCCGAGCAGGGCCGCATGACCAACAAGATGAAGAGC
ACCAAAGGCGCCCTGACCTTCAGCCCCTACCTGCTGAGCCACGTGATGGGCTACGGCTTC
TACCACTTCGGCACCTACCCCAGCGGCTACGAGAACCCCTTCCTGCACGCCATCAACAAC
GGCGGCTACACCAACACCCGCATCGAGAAGTACGAGGACGGCGGCGTGCTGCACGTGAGC
TTCAGCTACCGCTACGAGGCCGGCCGCGTGATCGGCGACTTCAAGGTGGTGGGCACCGGC
TTCCCCGAGGACAGCGTGATCTTCACCGACAAGATCATCCGCAGCAACGCCACCGTGGAG
CACCTGCACCCCATGGGCGATAACGTGCTGGTGGGCAGCTTCGCCCGCACCTTCAGCCTG
CGCGACGGCGGCTACTACAGCTTCGTGGTGGACAGCCACATGCACTTCAAGAGCGCCATC
CACCCCAGCATCCTGCAGAACGGGGGCCCCATGTTCGCCTTCCGCCGCGTGGAGGAGCTG
CACAGCAACACCGAGCTGGGCATCGTGGAGTACCAGCACGCCTTCAAGACCCCCATCGCC
TTCGCCAGATCTCGAGATATCAGCCATGGCTTCCCGCCGGCGGTGGCGGCGCAGGATGAT
GGCACGCTGCCCATGTCTTGTGCCCAGGAGAGCGGGATGGACCGTCACCCTGCAGCCTGT
GCTTCTGCTAGGATCAATGTGACCGGTGAGGGCAGAGGAAGTCTTCTAACATGCGGTGAC
GTGGAGGAGAATCCCGGCCCTTCCGGTATGGAAGACGCCAAAAACATAAAGAAAGGCCCG
GCGCCATTCTATCCGCTAGAGGATGGAACCGCTGGAGAGCAACTGCATAAGGCTATGAAG
AGATACGCCCTGGTTCCTGGAACAATTGCTTTTACAGATGCACATATCGAGGTGAACATC
ACGTACGCGGAATACTTCGAAATGTCCGTTCGGTTGGCAGAAGCTATGAAACGATATGGG
CTGAATACAAATCACAGAATCGTCGTATGCAGTGAAAACTCTCTTCAATTCTTTATGCCG
GTGTTGGGCGCGTTATTTATCGGAGTTGCAGTTGCGCCCGCGAACGACATTTATAATGAA
CGTGAATTGCTCAACAGTATGAACATTTCGCAGCCTACCGTAGTGTTTGTTTCCAAAAAG
GGGTTGCAAAAAATTTTGAACGTGCAAAAAAAATTACCAATAATCCAGAAAATTATTATC
ATGGATTCTAAAACGGATTACCAGGGATTTCAGTCGATGTACACGTTCGTCACATCTCAT
CTACCTCCCGGTTTTAATGAATACGATTTTGTACCAGAGTCCTTTGATCGTGACAAAACA
ATTGCACTGATAATGAACTCCTCTGGATCTACTGGGTTACCTAAGGGTGTGGCCCTTCCG
CATAGAACTGCCTGCGTCAGATTCTCGCATGCCAGAGATCCTATTTTTGGCAATCAAATC
ATTCCGGATACTGCGATTTTAAGTGTTGTTCCATTCCATCACGGTTTTGGAATGTTTACT
ACACTCGGATATTTGATATGTGGATTTCGAGTCGTCTTAATGTATAGATTTGAAGAAGAG
CTGTTTTTACGATCCCTTCAGGATTACAAAATTCAAAGTGCGTTGCTAGTACCAACCCTA
TTTTCATTCTTCGCCAAAAGCACTCTGATTGACAAATACGATTTATCTAATTTACACGAA
ATTGCTTCTGGGGGCGCACCTCTTTCGAAAGAAGTCGGGGAAGCGGTTGCAAAACGCTTC
CATCTTCCAGGGATACGACAAGGATATGGGCTCACTGAGACTACATCAGCTATTCTGATT
ACACCCGAGGGGGATGATAAACCGGGCGCGGTCGGTAAAGTTGTTCCATTTTTTGAAGCG
AAGGTTGTGGATCTGGATACCGGGAAAACGCTGGGCGTTAATCAGAGAGGCGAATTATGT
GTCAGAGGACCTATGATTATGTCCGGTTATGTAAACAATCCGGAAGCGACCAACGCCTTG
ATTGACAAGGATGGATGGCTACATTCTGGAGACATAGCTTACTGGGACGAAGACGAACAC
TTCTTCATAGTTGACCGCTTGAAGTCTTTAATTAAATACAAAGGATACCAGGTGGCCCCC
GCTGAATTGGAGTCGATATTGTTACAACACCCCAACATCTTCGACGCGGGCGTGGCAGGT
CTTCCCGACGATGACGCCGGTGAACTTCCCGCCGCCGTTGTTGTTTTGGAGCACGGAAAG
ACGATGACGGAAAAAGAGATCGTGGATTACGTCGCCAGTCAAGTAACAACCGCGAAAAAG
TTGCGCGGAGGAGTTGTGTTTGTGGACGAAGTACCGAAAGGTCTTACCGGAAAACTCGAC
GCAAGAAAAATCAGAGAGATCCTCATAAAGGCCAAGAAGGGCGGAAAGTCCAAATTGTAA
H1 promoter sequence:
(SEQ ID NO: 15)
GAACGCTGACGTCATCAACCCGCTCCAAGGAATCGCGGGCCCAGTGTCACTAGGCGGGAA
CACCCAGCGCGCGTGCGCCCTGGCAGGAAGATGGCTGTGAGGGACAGGGGAGTGGCGCCC
TGCAATATTTGCATGTCGCTATGTGTTCTGGGAAATCACCATAAACGTGAAATGTCTTTG
GATTTGGGAATCTTATAAGTTCTGTATGAGACCACTT

### LV FDPS GFP T2A Luc Construction:

The pGF-1 plasmid (System Biosciences) containing the CMV GFP T2A luciferase sequence was digested with ClaI and KPN1 and the LV-H1-shFDPS plasmid was digested with BstBI and KpnI restriction enzymes (NEB). The DNA was electrophoresed on a 1% agarose gel and the DNA fragments were extracted with a DNA gel extraction kit (Thermo Scientific). The two fragments were ligated with T4 DNA ligase (NEB) and transformed into STBL3 bacteria (Thermo Scientific). Plasmid DNA was extracted from bacteria with a plasmid DNA mini prep kit (Thermo Scientific) and the sequence was verified by DNA sequencing (Eurofins Genomics).

### Detailed Experimental Protocol

Day −19: 175 ml flask grown confluently yields 1.87×10⁷ ml of PC3 cells; 75 ml flask grown confluently yields 7.5×10⁶ ml of PC3 cells.

Day −7: Thaw and grow PC3 cells

Day −4: Material Preparation and Delivery. Prepare lenti-vector control and lenti-shRNA-FDPS transduced PC3 cells.
1. In a 75 ml of flask, 50% confluent PC3 cells, add 12 μl of lenti-control+8 μl of polybrene, incubate for 5 min. then mix with 4 ml of RPMI-10, and cover the surface of PC3 cells.
2. In a 75 ml of flask, 50% confluent PC3 cell, add 20 μl of lenti-FDPS+8 μl of polybrene, incubate for 5 min. then mix with 4 ml of RPMI-10, and cover the surface of PC3 cells.
3. Incubate transduced cells at 37° C. for 8 hr. Add 6 ml of RPMI-10 for overnight culture

Day −2: Trypsinize 75 ml transduced PC3 cells (confluent 7.5×10⁶ cells) and transfer to 175 ml Flask.

Day 0: Material Preparation and Delivery
1. Trypsinize the 80% confluent lenti-vector and lenti-FDPS transduced PC3 cells separately and count cells.
   lenti-vector: 1.5×10⁸ cells (50×3×10⁶/5 ml) 15 flask
   lenti-FDPS: 1.5×10⁸ cells (50×3×10⁶/5 ml) 20 flask
2. Resuspend lenti-vector and lenti-FDPS transduced PC3 cells in RPMI without FBS, make the final concentration in 3×10⁶ cells/100 μl
   Material: I) 5 ml of PC3-Lenti-vector cells (total 150×10⁶ cells) in RPMI without FBS; II) 5 ml of PC3-Lenti-FDPS cells (total 150×10⁶ cells) in RPMI without FBS.

Day 0: Subcutaneous injection of PC3 cells. Group I (2 NOD/SCID mice): 0.15 ml of PC3-Lenti-vector cells (0.1 mL of 3×10⁶ Lenti-vector in RPMI without FBS+0.05 mL of Matrigel) are subcutaneously inoculated into either the right or left flanks of mice (total 5 ml enough for 50 mice). Group II (3 NOD/SCID mice): 0.15 ml of PC3-Lenti-FDPS KD (0.1 mL of 3×10⁶ Lenti-vector in DMEM without FBS+0.05 mL of Matrigel) are subcutaneously inoculated either the right or left flanks of mice (total 5 ml enough for 50 mice).

Day 8: Monitor tumor. Tumor is palpable in the first few days after implantation. Determine tumor size by measuring the perpendicular diameters of tumor with calipers. Tumor size is calculating by following measurement: Tumor volume (mm³)=d² (d=the shortest diameter)×D/2 (D=the longest diameter). Perform bioluminescence imaging to demonstrate tumor location, size and photon intensity as a measure of lentivirus expression of the firefly luciferase gene.

Day 14: Intraperitoneal injection of 100 μg/ml of zoledronic acid (Zol) or PBS to mice when tumor size reaches 200˜300 mm³.

Day 22: Imaging study to measure tumor size.

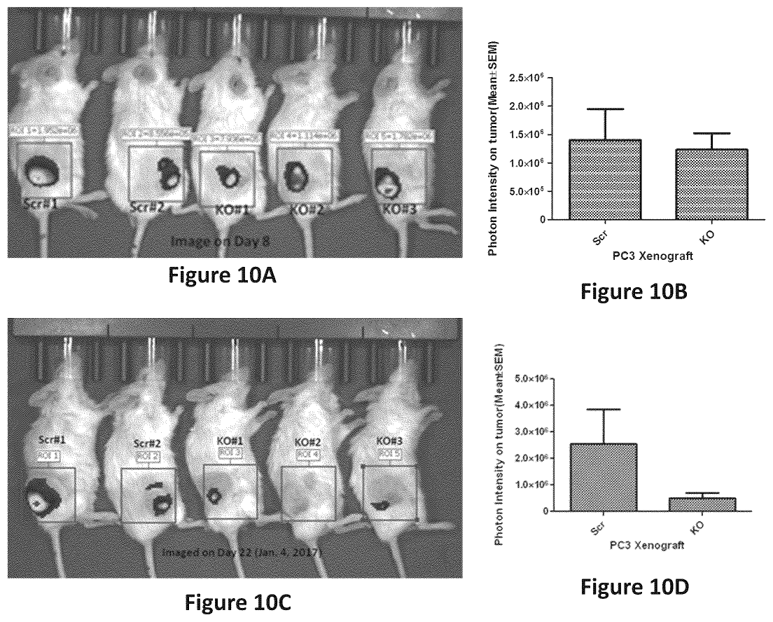

Effects of LV-shRNA-FDPS with or without zoledronic acid on PC3 tumor growth in NOD SCID mice. Mice were designated Scr (for scrambled vector control) or KO for LV-shRNA-FDPS. LV used for this study all express the bioluminescence marker firefly luciferase to enable direct visualization of transduced cells and their growth. A bioluminescence imaging study on Day 8 determined the average tumor sizes prior to zoledronic acid treatment (FIG. 10A). The photon intensity for tumors was measured with a CCD light capture system. The average size of tumor in the Scr animals was slightly larger than was found in the KO animals (FIG. 10B) but differences were not significant.

6 days after treatment with zoledronic acid (all animals received zoledronic acid by intraperitoneal injection), the imaging study was repeated. Tumor size and location for Scr animals (FIG. 10C) was similar to earlier observations but there were notable differences in tumor size for animals in the KO group. Tumor volume was reduced sharply in KO #1 and KO #3, and tumor was no longer present in KO #2. Comparing the average photon intensities for Scr and KO groups (FIG. 10D) revealed a substantial difference with the greatest change seen in the KO group.

These data show that LV-shRNA-FDPS has a small but detectable impact on growth of PC3 tumors in NOD/SCID mice. When combined with a single dose of zoledronic acid, the effect was magnified and eradication of LV-shRNA-FDPS transduced cells was achieved in one case. Thus, light-emitting transduced cells decreased by zoledronic acid only if the LV expressed a shRNA-FDPS. The reduction in tumor mass was not attributable to zoledronic acid treatment because animals with tumors transduced with scrambled control LV showed little or no change in tumor mass after zoledronic acid treatment.

The key to tumor reduction was the combined effect of LV-shRNA-FDPS reducing the levels of FDPS enzyme expression and zoledronic acid inhibiting any residual FDPS activity. As expected, the zoledronic acid was not toxic or mice and had no apparent effects other than reducing tumor mass when combined with LV-shRNA-FDPS. Zoledronic acid is a safe and effective treatment in humans where it is given in high bolus doses or as a chronic therapy for bone demineralization disorders including osteoporosis.

The disclosure of the example embodiments is intended to be illustrative, but not limiting, of the scope of the inventions, which are set forth in the following claims and their equivalents. Although example embodiments of the inventions have been described in some detail for purposes of clarity of understanding, it will be apparent that certain changes and modifications can be practiced within the scope of the following claims. In the following claims, elements and/or steps do not imply any particular order of operation, unless explicitly stated in the claims or implicitly required by the disclosure.

### Sequences

The following sequences are referred to herein:

| SEQ ID NO: | Description | Sequence |
|---|---|---|
| 1 | FDPS shRNA | GTCCTGGAGTACAATGCCATTCTCGAGAATGGCATTGT |
| | sequence #1 | ACTCCAGGACTTTTT |
| 2 | FDPS shRNA | GCAGGATTTCGTTCAGCACTTCTCGAGAAGTGCTGAAC |
| | sequence #2 | GAAATCCTGCTTTTT |
| 3 | FDPS shRNA | GCCATGTACATGGCAGGAATTCTCGAGAATTCCTGCCA |
| | sequence #3 | TGTACATGGCTTTTT |
| 4 | FDPS shRNA | GCAGAAGGAGGCTGAGAAAGTCTCGAGACTTTCTCAGC |
| | sequence #4 | CTCCTTCTGCTTTTT |
| 5 | CD47 shRNA | GGTGAAACGATCATCGAGCCTCGAGGCTCGATGATCGT |
| | sequence #1 | TTCACCTTTTT |
| 6 | CD47 shRNA | GCTACTGGCCTTGGTTTAACTCGAGTTAAACCAAGGCC |
| | sequence #2 | AGTAGCTTTTT |
| 7 | CD47 shRNA | CCTCCTTCGTCATTGCCATCTCGAGATGGCAATGACGA |
| | sequence #3 | AGGAGGTTTTT |
| 8 | CD47 shRNA | GCATGGCCCTCTTCTGATTCTCGAGAATCAGAAGAGGG |
| | sequence #4 | CCATGCTTTTT |
| 9 | CD47 shRNA | GGTGAAACGATCATCGAGCTACTCGAGTAGCTCGATGA |
| | sequence #5 | TCGTTTCACCTTTTT |
| 10 | cMyc shRNA | GCTTCACCAACAGGAACTATGCTCGAGCATAGTTCCTG |
| | sequence #1 | TTGGTGAAGCTTTT |
| 11 | cMyc shRNA | GCGAACACACAACGTCTTGGACTCGAGTCCAAGACGTT |
| | sequence #2 | GTGTGTTCGCTTTT |
| 12 | cMyc shRNA | GACATGGTGAACCAGAGTTTCCTCGAGGAAACTCTGGT |
| | sequence #3 | TCACCATGTCTTTTT |
| 13 | cMyc shRNA | GAGAATGTCAAGAGGCGAACACTCGAGTGTTCGCCTCT |
| | sequence #4 | TGACATTCTCTTTTT |
| 14 | cMyc shRNA | GCTCATTTCTGAAGAGGACTTCTCGAGAAGTCCTCTTC |
| | sequence #5 | AGAAATGAGCTTTTT |
| 15 | H1 promoter | GAACGCTGACGTCATCAACCCGCTCCAAGGAATCGCGG |
| | | GCCCAGTGTCACTAGGCGGGAACACCCAGCGCGCGTGC |
| | | GCCCTGGCAGGAAGATGGCTGTGAGGGACAGGGGAGTG |
| | | GCGCCCTGCAATATTTGCATGTCGCTATGTGTTCTGGG |
| | | AAATCACCATAAACGTGAAATGTCTTTGGATTTGGGAA |
| | | TCTTATAAGTTCTGTATGAGACCACTT |
| 16 | U6 promoter | GAGGGCCTATTTCCCATGATTCCTTCATATTTGCATAT |
| | | ACGATACAAGGCTGTTAGAGAGATAATTGGAATTAATT |
| | | TGACTGTAAACACAAAGATATTAGTACAAAATACGTGA |
| | | CGTAGAAAGTAATAATTTCTTGGGTAGTTTGCAGTTTT |
| | | AAAATTATGTTTTAAAATGGACTATCATATGCTTACCG |
| | | TAACTTGAAAGTATTTCGATTTCTTGGCTTTATATATC |
| | | TTGTGGAAAGGACGAAACACC |
| 17 | 7SK promoter | CTGCAGTATTTAGCATGCCCCACCCATCTGCAAGGCAT |
| | | TCTGGATAGTGTCAAAACAGCCGGAAATCAAGTCCGTT |
| | | TATCTCAAACTTTAGCATTTTGGGAATAAATGATATTT |
| | | GCTATGCTGGTTAAATTAGATTTTAGTTAAATTTCCTG |
| | | CTGAAGCTCTAGTACGATAAGCAACTTGACCTAAGTGT |
| | | AAAGTTGAGATTTCCTTCAGGTTTATATAGCTTGTGCG |
| | | CCGCCTGGCTACCTC |
| 18 | CAG enhancer | TAGTTATTAATAGTAATCAATTACGGGGTCATTAGTTC |
| | | ATAGCCCATATATGGAGTTCCGCGTTACATAACTTACG |
| | | GTAAATGGCCCGCCTGGCTGACCGCCCAACGACCCCCG |
| | | CCCATTGACGTCAATAATGACGTATGTTCCCATAGTAA |
| | | CGCCAATAGGGACTTTCCATTGACGTCAATGGGTGGAC |
| | | TATTTACGGTAAACTGCCCACTTGGCAGTACATCAAGT |
| | | GTATCATATGCCAAGTACGCCCCCTATTGACGTCAATG |
| | | ACGGTAAATGGCCCGCCTGGCATTATGCCCAGTACATG |
| | | ACCTTATGGGACTTTCCTACTTGGCAGTACATCTACGT |
| | | ATTAGTCATC |
| 19 | CAG promoter | GCTATTACCATGGGTCGAGGTGAGCCCCACGTTCTGCT |
| | | TCACTCTCCCCATCTCCCCCCCCTCCCCACCCCCAATT |
| | | TTGTATTTATTTATTTTTTAATTATTTTGTGCAGCGAT |
| | | GGGGGCGGGGGGGGGGGGGGCGCGCGCCAGGCGGGGCG |
| | | GGGCGGGGCGAGGGGCGGGGCGGGGCGAGGCGGAGAGG |
| | | TGCGGCGGCAGCCAATCAGAGCGGCGCGCTCCGAAAGT |
| | | TTCCTTTTATGGCGAGGCGGCGGCGGCGGCGGCCCTAT |
| | | AAAAAGCGAAGCGCGCGGCGGGCG |
| 20 | chicken beta | GGAGTCGCTGCGTTGCCTTCGCCCCGTGCCCCGCTCCG |
| | actin intron | CGCCGCCTCGCGCCGCCCGCCCCGGCTCTGACTGACCG |
| | | CGTTACTCCCACAGGTGAGCGGGCGGGACGGCCCTTCT |
| | | CCTCCGGGCTGTAATTAGCGCTTGGTTTAATGACGGCT |
| | | CGTTTCTTTTCTGTGGCTGCGTGAAAGCCTTAAAGGGC |
| | | TCCGGGAGGGCCCTTTGTGCGGGGGGGAGCGGCTCGGG |
| | | GGGTGCGTGCGTGTGTGTGTGCGTGGGGAGCGCCGCGT |
| | | GCGGCCCGCGCTGCCCGGCGGCTGTGAGCGCTGCGGGC |
| | | GCGGCGCGGGGCTTTGTGCGCTCCGCGTGTGCGCGAGG |
| | | GGAGCGCGGCCGGGGGCGGTGCCCCGCGGTGCGGGGGG |
| | | GCTGCGAGGGGAACAAAGGCTGCGTGCGGGGTGTGTGC |
| | | GTGGGGGGGTGAGCAGGGGGTGTGGGCGCGGCGGTCGG |
| | | GCTGTAACCCCCCCCTGCACCCCCCTCCCCGAGTTGCT |
| | | GAGCACGGCCCGGCTTCGGGTGCGGGGCTCCGTGCGGG |
| | | GCGTGGCGCGGGGCTCGCCGTGCCGGGCGGGGGGTGGC |
| | | GGCAGGTGGGGGTGCCGGGCGGGGCGGGGCCGCCTCGG |
| | | GCCGGGGAGGGCTCGGGGGAGGGGCGCGGCGGCCCCGG |
| | | AGCGCCGGCGGCTGTCGAGGCGCGGCGAGCCGCAGCCA |
| | | TTGCCTTTTATGGTAATCGTGCGAGAGGGCGCAGGGAC |
| | | TTCCTTTGTCCCAAATCTGGCGGAGCCGAAATCTGGGA |
| | | GGCGCCGCCGCACCCCCTCTAGCGGGCGCGGGCGAAGC |
| | | GGTGCGGCGCCGGCAGGAAGGAAATGGGCGGGGAGGGC |
| | | CTTCGTGCGTCGCCGCGCCGCCGTCCCCTTCTCCATCT |
| | | CCAGCCTCGGGGCTGCCGCAGGGGGACGGCTGCCTTCG |
| | | GGGGGGACGGGGCAGGGCGGGGTTCGGCTTCTGGCGTG |
| | | TGACCGGCGG |
| 21 | HIV gag | ATGGGTGCGAGAGCGTCAGTATTAAGCGGGGGAGAATT |
| | | AGATCGATGGGAAAAAATTCGGTTAAGGCCAGGGGGAA |
| | | AGAAAAAATATAAATTAAAACATATAGTATGGGCAAGC |
| | | AGGGAGCTAGAACGATTCGCAGTTAATCCTGGCCTGTT |
| | | AGAAACATCAGAAGGCTGTAGACAAATACTGGGACAGC |
| | | TACAACCATCCCTTCAGACAGGATCAGAAGAACTTAGA |
| | | TCATTATATAATACAGTAGCAACCCTCTATTGTGTGCA |
| | | TCAAAGGATAGAGATAAAAGACACCAAGGAAGCTTTAG |
| | | ACAAGATAGAGGAAGAGCAAAACAAAAGTAAGAAAAAA |
| | | GCACAGCAAGCAGCAGCTGACACAGGACACAGCAATCA |
| | | GGTCAGCCAAAATTACCCTATAGTGCAGAACATCCAGG |
| | | GGCAAATGGTACATCAGGCCATATCACCTAGAACTTTA |
| | | AATGCATGGGTAAAAGTAGTAGAAGAGAAGGCTTTCAG |
| | | CCCAGAAGTGATACCCATGTTTTCAGCATTATCAGAAG |
| | | GAGCCACCCCACAAGATTTAAACACCATGCTAAACACA |
| | | GTGGGGGGACATCAAGCAGCCATGCAAATGTTAAAAGA |
| | | GACCATCAATGAGGAAGCTGCAGAATGGGATAGAGTGC |
| | | ATCCAGTGCATGCAGGGCCTATTGCACCAGGCCAGATG |
| | | AGAGAACCAAGGGGAAGTGACATAGCAGGAACTACTAG |
| | | TACCCTTCAGGAACAAATAGGATGGATGACACATAATC |
| | | CACCTATCCCAGTAGGAGAAATCTATAAAAGATGGATA |
| | | ATCCTGGGATTAAATAAAATAGTAAGAATGTATAGCCC |
| | | TACCAGCATTCTGGACATAAGACAAGGACCAAAGGAAC |
| | | CCTTTAGAGACTATGTAGACCGATTCTATAAAACTCTA |
| | | AGAGCCGAGCAAGCTTCACAAGAGGTAAAAAATTGGAT |
| | | GACAGAAACCTTGTTGGTCCAAAATGCGAACCCAGATT |
| | | GTAAGACTATTTTAAAAGCATTGGGACCAGGAGCGACA |
| | | CTAGAAGAAATGATGACAGCATGTCAGGGAGTGGGGGG |
| | | ACCCGGCCATAAAGCAAGAGTTTTGGCTGAAGCAATGA |
| | | GCCAAGTAACAAATCCAGCTACCATAATGATACAGAAA |
| | | GGCAATTTTAGGAACCAAAGAAAGACTGTTAAGTGTTT |
| | | CAATTGTGGCAAAGAAGGGCACATAGCCAAAAATTGCA |
| | | GGGCCCCTAGGAAAAAGGGCTGTTGGAAATGTGGAAAG |
| | | GAAGGACACCAAATGAAAGATTGTACTGAGAGACAGGC |
| | | TAATTTTTTAGGGAAGATCTGGCCTTCCCACAAGGGAA |
| | | GGCCAGGGAATTTTCTTCAGAGCAGACCAGAGCCAACA |
| | | GCCCCACCAGAAGAGAGCTTCAGGTTTGGGGAAGAGAC |
| | | AACAACTCCCTCTCAGAAGCAGGAGCCGATAGACAAGG |
| | | AACTGTATCCTTTAGCTTCCCTCAGATCACTCTTTGGC |
| | | AGCGACCCCTCGTCACAATAA |
| 22 | HIV Pol | ATGAATTTGCCAGGAAGATGGAAACCAAAAATGATAGG |
| | | GGGAATTGGAGGTTTTATCAAAGTAGGACAGTATGATC |
| | | AGATACTCATAGAAATCTGCGGACATAAAGCTATAGGT |
| | | ACAGTATTAGTAGGACCTACACCTGTCAACATAATTGG |
| | | AAGAAATCTGTTGACTCAGATTGGCTGCACTTTAAATT |
| | | TTCCCATTAGTCCTATTGAGACTGTACCAGTAAAATTA |
| | | AAGCCAGGAATGGATGGCCCAAAAGTTAAACAATGGCC |
| | | ATTGACAGAAGAAAAAATAAAAGCATTAGTAGAAATTT |
| | | GTACAGAAATGGAAAAGGAAGGAAAAATTTCAAAAATT |
| | | GGGCCTGAAAATCCATACAATACTCCAGTATTTGCCAT |
| | | AAAGAAAAAAGACAGTACTAAATGGAGAAAATTAGTAG |
| | | ATTTCAGAGAACTTAATAAGAGAACTCAAGATTTCTGG |
| | | GAAGTTCAATTAGGAATACCACATCCTGCAGGGTTAAA |
| | | ACAGAAAAAATCAGTAACAGTACTGGATGTGGGCGATG |
| | | CATATTTTTCAGTTCCCTTAGATAAAGACTTCAGGAAG |
| | | TATACTGCATTTACCATACCTAGTATAAACAATGAGAC |
| | | ACCAGGGATTAGATATCAGTACAATGTGCTTCCACAGG |
| | | GATGGAAAGGATCACCAGCAATATTCCAGTGTAGCATG |
| | | ACAAAAATCTTAGAGCCTTTTAGAAAACAAAATCCAGA |
| | | CATAGTCATCTATCAATACATGGATGATTTGTATGTAG |
| | | GATCTGACTTAGAAATAGGGCAGCATAGAACAAAAATA |
| | | GAGGAACTGAGACAACATCTGTTGAGGTGGGGATTTAC |
| | | CACACCAGACAAAAAACATCAGAAAGAACCTCCATTCC |
| | | TTTGGATGGGTTATGAACTCCATCCTGATAAATGGACA |
| | | GTACAGCCTATAGTGCTGCCAGAAAAGGACAGCTGGAC |
| | | TGTCAATGACATACAGAAATTAGTGGGAAAATTGAATT |
| | | GGGCAAGTCAGATTTATGCAGGGATTAAAGTAAGGCAA |
| | | TTATGTAAACTTCTTAGGGGAACCAAAGCACTAACAGA |
| | | AGTAGTACCACTAACAGAAGAAGCAGAGCTAGAACTGG |
| | | CAGAAAACAGGGAGATTCTAAAAGAACCGGTACATGGA |
| | | GTGTATTATGACCCATCAAAAGACTTAATAGCAGAAAT |
| | | ACAGAAGCAGGGGCAAGGCCAATGGACATATCAAATTT |
| | | ATCAAGAGCCATTTAAAAATCTGAAAACAGGAAAATAT |
| | | GCAAGAATGAAGGGTGCCCACACTAATGATGTGAAACA |
| | | ATTAACAGAGGCAGTACAAAAAATAGCCACAGAAAGCA |
| | | TAGTAATATGGGGAAAGACTCCTAAATTTAAATTACCC |
| | | ATACAAAAGGAAACATGGGAAGCATGGTGGACAGAGTA |
| | | TTGGCAAGCCACCTGGATTCCTGAGTGGGAGTTTGTCA |
| | | ATACCCCTCCCTTAGTGAAGTTATGGTACCAGTTAGAG |
| | | AAAGAACCCATAATAGGAGCAGAAACTTTCTATGTAGA |
| | | TGGGGCAGCCAATAGGGAAACTAAATTAGGAAAAGCAG |
| | | GATATGTAACTGACAGAGGAAGACAAAAAGTTGTCCCC |
| | | CTAACGGACACAACAAATCAGAAGACTGAGTTACAAGC |
| | | AATTCATCTAGCTTTGCAGGATTCGGGATTAGAAGTAA |
| | | ACATAGTGACAGACTCACAATATGCATTGGGAATCATT |
| | | CAAGCACAACCAGATAAGAGTGAATCAGAGTTAGTCAG |
| | | TCAAATAATAGAGCAGTTAATAAAAAAGGAAAAAGTCT |
| | | ACCTGGCATGGGTACCAGCACACAAAGGAATTGGAGGA |
| | | AATGAACAAGTAGATGGGTTGGTCAGTGCTGGAATCAG |
| | | GAAAGTACTA |
| 23 | HIV Int | TTTTTAGATGGAATAGATAAGGCCCAAGAAGAACATGA |
| | | GAAATATCACAGTAATTGGAGAGCAATGGCTAGTGATT |
| | | TTAACCTACCACCTGTAGTAGCAAAAGAAATAGTAGCC |
| | | AGCTGTGATAAATGTCAGCTAAAAGGGGAAGCCATGCA |
| | | TGGACAAGTAGACTGTAGCCCAGGAATATGGCAGCTAG |
| | | ATTGTACACATTTAGAAGGAAAAGTTATCTTGGTAGCA |
| | | GTTCATGTAGCCAGTGGATATATAGAAGCAGAAGTAAT |
| | | TCCAGCAGAGACAGGGCAAGAAACAGCATACTTCCTCT |
| | | TAAAATTAGCAGGAAGATGGCCAGTAAAAACAGTACAT |
| | | ACAGACAATGGCAGCAATTTCACCAGTACTACAGTTAA |
| | | GGCCGCCTGTTGGTGGGCGGGGATCAAGCAGGAATTTG |
| | | GCATTCCCTACAATCCCCAAAGTCAAGGAGTAATAGAA |
| | | TCTATGAATAAAGAATTAAAGAAAATTATAGGACAGGT |
| | | AAGAGATCAGGCTGAACATCTTAAGACAGCAGTACAAA |
| | | TGGCAGTATTCATCCACAATTTTAAAAGAAAAGGGGGG |
| | | ATTGGGGGGTACAGTGCAGGGGAAAGAATAGTAGACAT |
| | | AATAGCAACAGACATACAAACTAAAGAATTACAAAAAC |
| | | AAATTACAAAAATTCAAAATTTTCGGGTTTATTACAGG |
| | | GACAGCAGAGATCCAGTTTGGAAAGGACCAGCAAAGCT |
| | | CCTCTGGAAAGGTGAAGGGGCAGTAGTAATACAAGATA |
| | | ATAGTGACATAAAAGTAGTGCCAAGAAGAAAAGCAAAG |
| | | ATCATCAGGGATTATGGAAAACAGATGGCAGGTGATGA |
| | | TTGTGTGGCAAGTAGACAGGATGAGGATTAA |
| 24 | HIV RRE | AGGAGCTTTGTTCCTTGGGTTCTTGGGAGCAGCAGGAA |
| | | GCACTATGGGCGCAGCGTCAATGACGCTGACGGTACAG |
| | | GCCAGACAATTATTGTCTGGTATAGTGCAGCAGCAGAA |
| | | CAATTTGCTGAGGGCTATTGAGGCGCAACAGCATCTGT |
| | | TGCAACTCACAGTCTGGGGCATCAAGCAGCTCCAGGCA |
| | | AGAATCCTGGCTGTGGAAAGATACCTAAAGGATCAACA |
| | | GCTCCT |
| 25 | HIV Rev | ATGGCAGGAAGAAGCGGAGACAGCGACGAAGAACTCCT |
| | | CAAGGCAGTCAGACTCATCAAGTTTCTCTATCAAAGCA |
| | | ACCCACCTCCCAATCCCGAGGGGACCCGACAGGCCCGA |
| | | AGGAATAGAAGAAGAAGGTGGAGAGAGAGACAGAGACA |
| | | GATCCATTCGATTAGTGAACGGATCCTTAGCACTTATC |
| | | TGGGACGATCTGCGGAGCCTGTGCCTCTTCAGCTACCA |
| | | CCGCTTGAGAGACTTACTCTTGATTGTAACGAGGATTG |
| | | TGGAACTTCTGGGACGCAGGGGGTGGGAAGCCCTCAAA |
| | | TATTGGTGGAATCTCCTACAATATTGGAGTCAGGAGCT |
| | | AAAGAATAG |
| 26 | rabbit beta | AGATCTTTTTCCCTCTGCCAAAAATTATGGGGACATCA |
| | globin poly A | TGAAGCCCCTTGAGCATCTGACTTCTGGCTAATAAAGG |
| | | AAATTTATTTTCATTGCAATAGTGTGTTGGAATTTTTT |
| | | GTGTCTCTCACTCGGAAGGACATATGGGAGGGCAAATC |
| | | ATTTAAAACATCAGAATGAGTATTTGGTTTAGAGTTTG |
| | | GCAACATATGCCATATGCTGGCTGCCATGAACAAAGGT |
| | | GGCTATAAAGAGGTCATCAGTATATGAAACAGCCCCCT |
| | | GCTGTCCATTCCTTATTCCATAGAAAAGCCTTGACTTG |
| | | AGGTTAGATTTTTTTTATATTTTGTTTTGTGTTATTTT |
| | | TTTCTTTAACATCCCTAAAATTTTCCTTACATGTTTTA |
| | | CTAGCCAGATTTTTCCTCCTCTCCTGACTACTCCCAGT |
| | | CATAGCTGTCCCTCTTCTCTTATGAAGATC |
| 27 | CMV | ACATTGATTATTGACTAGTTATTAATAGTAATCAATTA |
| | Promoter | CGGGGTCATTAGTTCATAGCCCATATATGGAGTTCCGC |
| | | GTTACATAACTTACGGTAAATGGCCCGCCTGGCTGACC |
| | | GCCCAACGACCCCCGCCCATTGACGTCAATAATGACGT |
| | | ATGTTCCCATAGTAACGCCAATAGGGACTTTCCATTGA |
| | | CGTCAATGGGTGGAGTATTTACGGTAAACTGCCCACTT |
| | | GGCAGTACATCAAGTGTATCATATGCCAAGTACGCCCC |
| | | CTATTGACGTCAATGACGGTAAATGGCCCGCCTGGCAT |
| | | TATGCCCAGTACATGACCTTATGGGACTTTCCTACTTG |
| | | GCAGTACATCTACGTATTAGTCATCGCTATTACCATGG |
| | | TGATGCGGTTTTGGCAGTACATCAATGGGCGTGGATAG |
| | | CGGTTTGACTCACGGGGATTTCCAAGTCTCCACCCCAT |
| | | TGACGTCAATGGGAGTTTGTTTTGGCACCAAAATCAAC |
| | | GGGACTTTCCAAAATGTCGTAACAACTCCGCCCCATTG |
| | | ACGCAAATGGGCGGTAGGCGTGTACGGTGGGAGGTCTA |
| | | TATAAGC |
| 28 | beta globin | GTGAGTTTGGGGACCCTTGATTGTTCTTTCTTTTTCGC |
| | intron | TATTGTAAAATTCATGTTATATGGAGGGGGCAAAGTTT |
| | | TCAGGGTGTTGTTTAGAATGGGAAGATGTCCCTTGTAT |
| | | CACCATGGACCCTCATGATAATTTTGTTTCTTTCACTT |
| | | TCTACTCTGTTGACAACCATTGTCTCCTCTTATTTTCT |
| | | TTTCATTTTCTGTAACTTTTTCGTTAAACTTTAGCTTG |
| | | CATTTGTAACGAATTTTTAAATTCACTTTTGTTTATTT |
| | | GTCAGATTGTAAGTACTTTCTCTAATCACTTTTTTTTC |
| | | AAGGCAATCAGGGTATATTATATTGTACTTCAGCACAG |
| | | TTTTAGAGAACAATTGTTATAATTAAATGATAAGGTAG |
| | | AATATTTCTGCATATAAATTCTGGCTGGCGTGGAAATA |
| | | TTCTTATTGGTAGAAACAACTACACCCTGGTCATCATC |
| | | CTGCCTTTCTCTTTATGGTTACAATGATATACACTGTT |
| | | TGAGATGAGGATAAAATACTCTGAGTCCAAACCGGGCC |
| | | CCTCTGCTAACCATGTTCATGCCTTCTTCTCTTTCCTA |
| | | CAG |
| 29 | VSV-G/DNA | GAATTCATGAAGTGCCTTTTGTACTTAGCCTTTTTATT |
| | fragment | CATTGGGGTGAATTGCAAGTTCACCATAGTTTTTCCAC |
| | containing | ACAACCAAAAAGGAAACTGGAAAAATGTTCCTTCTAAT |
| | VSV-G | TACCATTATTGCCCGTCAAGCTCAGATTTAAATTGGCA |
| | | TAATGACTTAATAGGCACAGCCTTACAAGTCAAAATGC |
| | | CCAAGAGTCACAAGGCTATTCAAGCAGACGGTTGGATG |
| | | TGTCATGCTTCCAAATGGGTCACTACTTGTGATTTCCG |
| | | CTGGTATGGACCGAAGTATATAACACATTCCATCCGAT |
| | | CCTTCACTCCATCTGTAGAACAATGCAAGGAAAGCATT |
| | | GAACAAACGAAACAAGGAACTTGGCTGAATCCAGGCTT |
| | | CCCTCCTCAAAGTTGTGGATATGCAACTGTGACGGATG |
| | | CCGAAGCAGTGATTGTCCAGGTGACTCCTCACCATGTG |
| | | CTGGTTGATGAATACACAGGAGAATGGGTTGATTCACA |
| | | GTTCATCAACGGAAAATGCAGCAATTACATATGCCCCA |
| | | CTGTCCATAACTCTACAACCTGGCATTCTGACTATAAG |
| | | GTCAAAGGGCTATGTGATTCTAACCTCATTTCCATGGA |
| | | CATCACCTTCTTCTCAGAGGACGGAGAGCTATCATCCC |
| | | TGGGAAAGGAGGGCACAGGGTTCAGAAGTAACTACTTT |
| | | GCTTATGAAACTGGAGGCAAGGCCTGCAAAATGCAATA |
| | | CTGCAAGCATTGGGGAGTCAGACTCCCATCAGGTGTCT |
| | | GGTTCGAGATGGCTGATAAGGATCTCTTTGCTGCAGCC |
| | | AGATTCCCTGAATGCCCAGAAGGGTCAAGTATCTCTGC |
| | | TCCATCTCAGACCTCAGTGGATGTAAGTCTAATTCAGG |
| | | ACGTTGAGAGGATCTTGGATTATTCCCTCTGCCAAGAA |
| | | ACCTGGAGCAAAATCAGAGCGGGTCTTCCAATCTCTCC |
| | | AGTGGATCTCAGCTATCTTGCTCCTAAAAACCCAGGAA |
| | | CCGGTCCTGCTTTCACCATAATCAATGGTACCCTAAAA |
| | | TACTTTGAGACCAGATACATCAGAGTCGATATTGCTGC |
| | | TCCAATCCTCTCAAGAATGGTCGGAATGATCAGTGGAA |
| | | CTACCACAGAAAGGGAACTGTGGGATGACTGGGCACCA |
| | | TATGAAGACGTGGAAATTGGACCCAATGGAGTTCTGAG |
| | | GACCAGTTCAGGATATAAGTTTCCTTTATACATGATTG |
| | | GACATGGTATGTTGGACTCCGATCTTCATCTTAGCTCA |
| | | AAGGCTCAGGTGTTCGAACATCCTCACATTCAAGACGC |
| | | TGCTTCGCAACTTCCTGATGATGAGAGTTTATTTTTTG |
| | | GTGATACTGGGCTATCCAAAAATCCAATCGAGCTTGTA |
| | | GAAGGTTGGTTCAGTAGTTGGAAAAGCTCTATTGCCTC |
| | | TTTTTTCTTTATCATAGGGTTAATCATTGGACTATTCT |
| | | TGGTTCTCCGAGTTGGTATCCATCTTTGCATTAAATTA |
| | | AAGCACACCAAGAAAAGACAGATTTATACAGACATAGA |
| | | GATGAGAATTC |
| 30 | rabbit beta | AGATCTTTTTCCCTCTGCCAAAAATTATGGGGACATCA |
| | globin poly A | TGAAGCCCCTTGAGCATCTGACTTCTGGCTAATAAAGG |
| | | AAATTTATTTTCATTGCAATAGTGTGTTGGAATTTTTT |
| | | GTGTCTCTCACTCGGAAGGACATATGGGAGGGCAAATC |
| | | ATTTAAAACATCAGAATGAGTATTTGGTTTAGAGTTTG |
| | | GCAACATATGCCCATATGCTGGCTGCCATGAACAAAGG |
| | | TTGGCTATAAAGAGGTCATCAGTATATGAAACAGCCCC |
| | | CTGCTGTCCATTCCTTATTCCATAGAAAAGCCTTGACT |
| | | TGAGGTTAGATTTTTTTTATATTTTGTTTTGTGTTATT |
| | | TTTTTCTTTAACATCCCTAAAATTTTCCTTACATGTTT |
| | | TACTAGCCAGATTTTTCCTCCTCTCCTGACTACTCCCA |
| | | GTCATAGCTGTCCCTCTTCTCTTATGGAGATC |
| 31 | Primer | TAAGCAGAATTCATGAATTTGCCAGGAAGAT |
| 32 | Primer | CCATACAATGAATGGACACTAGGCGGCCGCACGAAT |
| 33 | Gag, Pol, | GAATTCATGAATTTGCCAGGAAGATGGAAACCAAAAAT |
| | Integrase | GATAGGGGGAATTGGAGGTTTTATCAAAGTAAGACAGT |
| | fragment | ATGATCAGATACTCATAGAAATCTGCGGACATAAAGCT |
| | | ATAGGTACAGTATTAGTAGGACCTACACCTGTCAACAT |
| | | AATTGGAAGAAATCTGTTGACTCAGATTGGCTGCACTT |
| | | TAAATTTTCCCATTAGTCCTATTGAGACTGTACCAGTA |
| | | AAATTAAAGCCAGGAATGGATGGCCCAAAAGTTAAACA |
| | | ATGGCCATTGACAGAAGAAAAAATAAAAGCATTAGTAG |
| | | AAATTTGTACAGAAATGGAAAAGGAAGGAAAAATTTCA |
| | | AAAATTGGGCCTGAAAATCCATACAATACTCCAGTATT |
| | | TGCCATAAAGAAAAAAGACAGTACTAAATGGAGAAAAT |
| | | TAGTAGATTTCAGAGAACTTAATAAGAGAACTCAAGAT |
| | | TTCTGGGAAGTTCAATTAGGAATACCACATCCTGCAGG |
| | | GTTAAAACAGAAAAAATCAGTAACAGTACTGGATGTGG |
| | | GCGATGCATATTTTTCAGTTCCCTTAGATAAAGACTTC |
| | | AGGAAGTATACTGCATTTACCATACCTAGTATAAACAA |
| | | TGAGACACCAGGGATTAGATATCAGTACAATGTGCTTC |
| | | CACAGGGATGGAAAGGATCACCAGCAATATTCCAGTGT |
| | | AGCATGACAAAAATCTTAGAGCCTTTTAGAAAACAAAA |
| | | TCCAGACATAGTCATCTATCAATACATGGATGATTTGT |
| | | ATGTAGGATCTGACTTAGAAATAGGGCAGCATAGAACA |
| | | AAAATAGAGGAACTGAGACAACATCTGTTGAGGTGGGG |
| | | ATTTACCACACCAGACAAAAAACATCAGAAAGAACCTC |
| | | CATTCCTTTGGATGGGTTATGAACTCCATCCTGATAAA |
| | | TGGACAGTACAGCCTATAGTGCTGCCAGAAAAGGACAG |
| | | CTGGACTGTCAATGACATACAGAAATTAGTGGGAAAAT |
| | | TGAATTGGGCAAGTCAGATTTATGCAGGGATTAAAGTA |
| | | AGGCAATTATGTAAACTTCTTAGGGGAACCAAAGCACT |
| | | AACAGAAGTAGTACCACTAACAGAAGAAGCAGAGCTAG |
| | | AACTGGCAGAAAACAGGGAGATTCTAAAAGAACCGGTA |
| | | CATGGAGTGTATTATGACCCATCAAAAGACTTAATAGC |
| | | AGAAATACAGAAGCAGGGGCAAGGCCAATGGACATATC |
| | | AAATTTATCAAGAGCCATTTAAAAATCTGAAAACAGGA |
| | | AAGTATGCAAGAATGAAGGGTGCCCACACTAATGATGT |
| | | GAAACAATTAACAGAGGCAGTACAAAAAATAGCCACAG |
| | | AAAGCATAGTAATATGGGGAAAGACTCCTAAATTTAAA |
| | | TTACCCATACAAAAGGAAACATGGGAAGCATGGTGGAC |
| | | AGAGTATTGGCAAGCCACCTGGATTCCTGAGTGGGAGT |
| | | TTGTCAATACCCCTCCCTTAGTGAAGTTATGGTACCAG |
| | | TTAGAGAAAGAACCCATAATAGGAGCAGAAACTTTCTA |
| | | TGTAGATGGGGCAGCCAATAGGGAAACTAAATTAGGAA |
| | | AAGCAGGATATGTAACTGACAGAGGAAGACAAAAAGTT |
| | | GTCCCCCTAACGGACACAACAAATCAGAAGACTGAGTT |
| | | ACAAGCAATTCATCTAGCTTTGCAGGATTCGGGATTAG |
| | | AAGTAAACATAGTGACAGACTCACAATATGCATTGGGA |
| | | ATCATTCAAGCACAACCAGATAAGAGTGAATCAGAGTT |
| | | AGTCAGTCAAATAATAGAGCAGTTAATAAAAAAGGAAA |
| | | AAGTCTACCTGGCATGGGTACCAGCACACAAAGGAATT |
| | | GGAGGAAATGAACAAGTAGATAAATTGGTCAGTGCTGG |
| | | AATCAGGAAAGTACTATTTTTAGATGGAATAGATAAGG |
| | | CCCAAGAAGAACATGAGAAATATCACAGTAATTGGAGA |
| | | GCAATGGCTAGTGATTTTAACCTACCACCTGTAGTAGC |
| | | AAAAGAAATAGTAGCCAGCTGTGATAAATGTCAGCTAA |
| | | AAGGGGAAGCCATGCATGGACAAGTAGACTGTAGCCCA |
| | | GGAATATGGCAGCTAGATTGTACACATTTAGAAGGAAA |
| | | AGTTATCTTGGTAGCAGTTCATGTAGCCAGTGGATATA |
| | | TAGAAGCAGAAGTAATTCCAGCAGAGACAGGGCAAGAA |
| | | ACAGCATACTTCCTCTTAAAATTAGCAGGAAGATGGCC |
| | | AGTAAAAACAGTACATACAGACAATGGCAGCAATTTCA |
| | | CCAGTACTACAGTTAAGGCCGCCTGTTGGTGGGCGGGG |
| | | ATCAAGCAGGAATTTGGCATTCCCTACAATCCCCAAAG |
| | | TCAAGGAGTAATAGAATCTATGAATAAAGAATTAAAGA |
| | | AAATTATAGGACAGGTAAGAGATCAGGCTGAACATCTT |
| | | AAGACAGCAGTACAAATGGCAGTATTCATCCACAATTT |
| | | TAAAAGAAAAGGGGGGATTGGGGGGTACAGTGCAGGGG |
| | | AAAGAATAGTAGACATAATAGCAACAGACATACAAACT |
| | | AAAGAATTACAAAAACAAATTACAAAAATTCAAAATTT |
| | | TCGGGTTTATTACAGGGACAGCAGAGATCCAGTTTGGA |
| | | AAGGACCAGCAAAGCTCCTCTGGAAAGGTGAAGGGGCA |
| | | GTAGTAATACAAGATAATAGTGACATAAAAGTAGTGCC |
| | | AAGAAGAAAAGCAAAGATCATCAGGGATTATGGAAAAC |
| | | AGATGGCAGGTGATGATTGTGTGGCAAGTAGACAGGAT |
| | | GAGGATTAA |
| 34 | DNA | TCTAGAATGGCAGGAAGAAGCGGAGACAGCGACGAAGA |
| | Fragment | GCTCATCAGAACAGTCAGACTCATCAAGCTTCTCTATC |
| | containing | AAAGCAACCCACCTCCCAATCCCGAGGGGACCCGACAG |
| | Rev, RRE and | GCCCGAAGGAATAGAAGAAGAAGGTGGAGAGAGAGACA |
| | rabbit beta | GAGACAGATCCATTCGATTAGTGAACGGATCCTTGGCA |
| | globin poly A | CTTATCTGGGACGATCTGCGGAGCCTGTGCCTCTTCAG |
| | | CTACCACCGCTTGAGAGACTTACTCTTGATTGTAACGA |
| | | GGATTGTGGAACTTCTGGGACGCAGGGGGTGGGAAGCC |
| | | CTCAAATATTGGTGGAATCTCCTACAATATTGGAGTCA |
| | | GGAGCTAAAGAATAGAGGAGCTTTGTTCCTTGGGTTCT |
| | | TGGGAGCAGCAGGAAGCACTATGGGCGCAGCGTCAATG |
| | | ACGCTGACGGTACAGGCCAGACAATTATTGTCTGGTAT |
| | | AGTGCAGCAGCAGAACAATTTGCTGAGGGCTATTGAGG |
| | | CGCAACAGCATCTGTTGCAACTCACAGTCTGGGGCATC |
| | | AAGCAGCTCCAGGCAAGAATCCTGGCTGTGGAAAGATA |
| | | CCTAAAGGATCAACAGCTCCTAGATCTTTTTCCCTCTG |
| | | CCAAAAATTATGGGGACATCATGAAGCCCCTTGAGCAT |
| | | CTGACTTCTGGCTAATAAAGGAAATTTATTTTCATTGC |
| | | AATAGTGTGTTGGAATTTTTTGTGTCTCTCACTCGGAA |
| | | GGACATATGGGAGGGCAAATCATTTAAAACATCAGAAT |
| | | GAGTATTTGGTTTAGAGTTTGGCAACATATGCCATATG |
| | | CTGGCTGCCATGAACAAAGGTGGCTATAAAGAGGTCAT |
| | | CAGTATATGAAACAGCCCCCTGCTGTCCATTCCTTATT |
| | | CCATAGAAAAGCCTTGACTTGAGGTTAGATTTTTTTTA |
| | | TATTTTGTTTTGTGTTATTTTTTTCTTTAACATCCCTA |
| | | AAATTTTCCTTACATGTTTTACTAGCCAGATTTTTCCT |
| | | CCTCTCCTGACTACTCCCAGTCATAGCTGTCCCTCTTC |
| | | TCTTATGAAGATCCCTCGACCTGCAGCCCAAGCTTGGC |
| | | GTAATCATGGTCATAGCTGTTTCCTGTGTGAAATTGTT |
| | | ATCCGCTCACAATTCCACACAACATACGAGCCGGAAGC |
| | | ATAAAGTGTAAAGCCTGGGGTGCCTAATGAGTGAGCTA |
| | | ACTCACATTAATTGCGTTGCGCTCACTGCCCGCTTTCC |
| | | AGTCGGGAAACCTGTCGTGCCAGCGGATCCGCATCTCA |
| | | ATTAGTCAGCAACCATAGTCCCGCCCCTAACTCCGCCC |
| | | ATCCCGCCCCTAACTCCGCCCAGTTCCGCCCATTCTCC |
| | | GCCCCATGGCTGACTAATTTTTTTTATTTATGCAGAGG |
| | | CCGAGGCCGCCTCGGCCTCTGAGCTATTCCAGAAGTAG |
| | | TGAGGAGGCTTTTTTGGAGGCCTAGGCTTTTGCAAAAA |
| | | GCTAACTTGTTTATTGCAGCTTATAATGGTTACAAATA |
| | | AAGCAATAGCATCACAAATTTCACAAATAAAGCATTTT |
| | | TTTCACTGCATTCTAGTTGTGGTTTGTCCAAACTCATC |
| | | AATGTATCTTATCAGCGGCCGCCCCGGG |
| 35 | DNA fragment | ACGCGTTAGTTATTAATAGTAATCAATTACGGGGTCAT |
| | containing the | TAGTTCATAGCCCATATATGGAGTTCCGCGTTACATAA |
| | CAG | CTTACGGTAAATGGCCCGCCTGGCTGACCGCCCAACGA |
| | enhancer/ | CCCCCGCCCATTGACGTCAATAATGACGTATGTTCCCA |
| | promoter/ | TAGTAACGCCAATAGGGACTTTCCATTGACGTCAATGG |
| | intron | GTGGACTATTTACGGTAAACTGCCCACTTGGCAGTACA |
| | sequence | TCAAGTGTATCATATGCCAAGTACGCCCCCTATTGACG |
| | | TCAATGACGGTAAATGGCCCGCCTGGCATTATGCCCAG |
| | | TACATGACCTTATGGGACTTTCCTACTTGGCAGTACAT |
| | | CTACGTATTAGTCATCGCTATTACCATGGGTCGAGGTG |
| | | AGCCCCACGTTCTGCTTCACTCTCCCCATCTCCCCCCC |
| | | CTCCCCACCCCCAATTTTGTATTTATTTATTTTTTAAT |
| | | TATTTTGTGCAGCGATGGGGGCGGGGGGGGGGGGGGCG |
| | | CGCGCCAGGCGGGGCGGGGCGGGGCGAGGGGCGGGGCG |
| | | GGGCGAGGCGGAGAGGTGCGGCGGCAGCCAATCAGAGC |
| | | GGCGCGCTCCGAAAGTTTCCTTTTATGGCGAGGCGGCG |
| | | GCGGCGGCGGCCCTATAAAAAGCGAAGCGCGCGGCGGG |
| | | CGGGAGTCGCTGCGTTGCCTTCGCCCCGTGCCCCGCTC |
| | | CGCGCCGCCTCGCGCCGCCCGCCCCGGCTCTGACTGAC |
| | | CGCGTTACTCCCACAGGTGAGCGGGCGGGACGGCCCTT |
| | | CTCCTCCGGGCTGTAATTAGCGCTTGGTTTAATGACGG |
| | | CTCGTTTCTTTTCTGTGGCTGCGTGAAAGCCTTAAAGG |
| | | GCTCCGGGAGGGCCCTTTGTGCGGGGGGGAGCGGCTCG |
| | | GGGGGTGCGTGCGTGTGTGTGTGCGTGGGGAGCGCCGC |
| | | GTGCGGCCCGCGCTGCCCGGCGGCTGTGAGCGCTGCGG |
| | | GCGCGGCGCGGGGCTTTGTGCGCTCCGCGTGTGCGCGA |
| | | GGGGAGCGCGGCCGGGGGCGGTGCCCCGCGGTGCGGGG |
| | | GGGCTGCGAGGGGAACAAAGGCTGCGTGCGGGGTGTGT |
| | | GCGTGGGGGGGTGAGCAGGGGGTGTGGGCGCGGCGGTC |
| | | GGGCTGTAACCCCCCCCTGCACCCCCCTCCCCGAGTTG |
| | | CTGAGCACGGCCCGGCTTCGGGTGCGGGGCTCCGTGCG |
| | | GGGCGTGGCGCGGGGCTCGCCGTGCCGGGCGGGGGGTG |
| | | GCGGCAGGTGGGGGTGCCGGGCGGGGCGGGGCCGCCTC |
| | | GGGCCGGGGAGGGCTCGGGGGAGGGGCGCGGCGGCCCC |
| | | GGAGCGCCGGCGGCTGTCGAGGCGCGGCGAGCCGCAGC |
| | | CATTGCCTTTTATGGTAATCGTGCGAGAGGGCGCAGGG |
| | | ACTTCCTTTGTCCCAAATCTGGCGGAGCCGAAATCTGG |
| | | GAGGCGCCGCCGCACCCCCTCTAGCGGGCGCGGGCGAA |
| | | GCGGTGCGGCGCCGGCAGGAAGGAAATGGGCGGGGAGG |
| | | GCCTTCGTGCGTCGCCGCGCCGCCGTCCCCTTCTCCAT |
| | | CTCCAGCCTCGGGGCTGCCGCAGGGGGACGGCTGCCTT |
| | | CGGGGGGGACGGGGCAGGGCGGGGTTCGGCTTCTGGCG |
| | | TGTGACCGGCGGGAATTC |
| 36 | RSV promoter | CAATTGCGATGTACGGGCCAGATATACGCGTATCTGAG |
| | and HIV Rev | GGGACTAGGGTGTGTTTAGGCGAAAAGCGGGGCTTCGG |
| | | TTGTACGCGGTTAGGAGTCCCCTCAGGATATAGTAGTT |
| | | TCGCTTTTGCATAGGGAGGGGGAAATGTAGTCTTATGC |
| | | AATACACTTGTAGTCTTGCAACATGGTAACGATGAGTT |
| | | AGCAACATGCCTTACAAGGAGAGAAAAAGCACCGTGCA |
| | | TGCCGATTGGTGGAAGTAAGGTGGTACGATCGTGCCTT |
| | | ATTAGGAAGGCAACAGACAGGTCTGACATGGATTGGAC |
| | | GAACCACTGAATTCCGCATTGCAGAGATAATTGTATTT |
| | | AAGTGCCTAGCTCGATACAATAAACGCCATTTGACCAT |
| | | TCACCACATTGGTGTGCACCTCCAAGCTCGAGCTCGTT |
| | | TAGTGAACCGTCAGATCGCCTGGAGACGCCATCCACGC |
| | | TGTTTTGACCTCCATAGAAGACACCGGGACCGATCCAG |
| | | CCTCCCCTCGAAGCTAGCGATTAGGCATCTCCTATGGC |
| | | AGGAAGAAGCGGAGACAGCGACGAAGAACTCCTCAAGG |
| | | CAGTCAGACTCATCAAGTTTCTCTATCAAAGCAACCCA |
| | | CCTCCCAATCCCGAGGGGACCCGACAGGCCCGAAGGAA |
| | | TAGAAGAAGAAGGTGGAGAGAGAGACAGAGACAGATCC |
| | | ATTCGATTAGTGAACGGATCCTTAGCACTTATCTGGGA |
| | | CGATCTGCGGAGCCTGTGCCTCTTCAGCTACCACCGCT |
| | | TGAGAGACTTACTCTTGATTGTAACGAGGATTGTGGAA |
| | | CTTCTGGGACGCAGGGGGTGGGAAGCCCTCAAATATTG |
| | | GTGGAATCTCCTACAATATTGGAGTCAGGAGCTAAAGA |
| | | ATAGTCTAGA |
| 37 | Elongation | CCGGTGCCTAGAGAAGGTGGCGCGGGGTAAACTGGGAA |
| | Factor-1 alpha | AGTGATGTCGTGTACTGGCTCCGCCTTTTTCCCGAGGG |
| | (EF1-alpha) | TGGGGGAGAACCGTATATAAGTGCAGTAGTCGCCGTGA |
| | promoter | ACGTTCTTTTTCGCAACGGGTTTGCCGCCAGAACACAG |
| | | GTAAGTGCCGTGTGTGGTTCCCGCGGGCCTGGCCTCTT |
| | | TACGGGTTATGGCCCTTGCGTGCCTTGAATTACTTCCA |
| | | CGCCCCTGGCTGCAGTACGTGATTCTTGATCCCGAGCT |
| | | TCGGGTTGGAAGTGGGTGGGAGAGTTCGAGGCCTTGCG |
| | | CTTAAGGAGCCCCTTCGCCTCGTGCTTGAGTTGAGGCC |
| | | TGGCCTGGGCGCTGGGGCCGCCGCGTGCGAATCTGGTG |
| | | GCACCTTCGCGCCTGTCTCGCTGCTTTCGATAAGTCTC |
| | | TAGCCATTTAAAATTTTTGATGACCTGCTGCGACGCTT |
| | | TTTTTCTGGCAAGATAGTCTTGTAAATGCGGGCCAAGA |
| | | TCTGCACACTGGTATTTCGGTTTTTGGGGCCGCGGGCG |
| | | GCGACGGGGCCCGTGCGTCCCAGCGCACATGTTCGGCG |
| | | AGGCGGGGCCTGCGAGCGCGGCCACCGAGAATCGGACG |
| | | GGGGTAGTCTCAAGCTGGCCGGCCTGCTCTGGTGCCTG |
| | | GCCTCGCGCCGCCGTGTATCGCCCCGCCCTGGGCGGCA |
| | | AGGCTGGCCCGGTCGGCACCAGTTGCGTGAGCGGAAAG |
| | | ATGGCCGCTTCCCGGCCCTGCTGCAGGGAGCTCAAAAT |
| | | GGAGGACGCGGCGCTCGGGAGAGCGGGCGGGTGAGTCA |
| | | CCCACACAAAGGAAAAGGGCCTTTCCGTCCTCAGCCGT |
| | | CGCTTCATGTGACTCCACGGAGTACCGGGCGCCGTCCA |
| | | GGCACCTCGATTAGTTCTCGAGCTTTTGGAGTACGTCG |
| | | TCTTTAGGTTGGGGGGAGGGGTTTTATGCGATGGAGTT |
| | | TCCCCACACTGAGTGGGTGGAGACTGAAGTTAGGCCAG |
| | | CTTGGCACTTGATGTAATTCTCCTTGGAATTTGCCCTT |
| | | TTTGAGTTTGGATCTTGGTTCATTCTCAAGCCTCAGAC |
| | | AGTGGTTCAAAGTTTTTTTCTTCCATTTCAGGTGTCGT |
| | | GA |
| 38 | Promoter; | GGGGTTGGGGTTGCGCCTTTTCCAAGGCAGCCCTGGGT |
| | PGK | TTGCGCAGGGACGCGGCTGCTCTGGGCGTGGTTCCGGG |
| | | AAACGCAGCGGCGCCGACCCTGGGTCTCGCACATTCTT |
| | | CACGTCCGTTCGCAGCGTCACCCGGATCTTCGCCGCTA |
| | | CCCTTGTGGGCCCCCCGGCGACGCTTCCTGCTCCGCCC |
| | | CTAAGTCGGGAAGGTTCCTTGCGGTTCGCGGCGTGCCG |
| | | GACGTGACAAACGGAAGCCGCACGTCTCACTAGTACCC |
| | | TCGCAGACGGACAGCGCCAGGGAGCAATGGCAGCGCGC |
| | | CGACCGCGATGGGCTGTGGCCAATAGCGGCTGCTCAGC |
| | | AGGGCGCGCCGAGAGCAGCGGCCGGGAAGGGGCGGTGC |
| | | GGGAGGCGGGGTGTGGGGCGGTAGTGTGGGCCCTGTTC |
| | | CTGCCCGCGCGGTGTTCCGCATTCTGCAAGCCTCCGGA |
| | | GCGCACGTCGGCAGTCGGCTCCCTCGTTGACCGAATCA |
| | | CCGACCTCTCTCCCCAG |
| 39 | Promoter; UbC | GCGCCGGGTTTTGGCGCCTCCCGCGGGCGCCCCCCTCC |
| | | TCACGGCGAGCGCTGCCACGTCAGACGAAGGGCGCAGG |
| | | AGCGTTCCTGATCCTTCCGCCCGGACGCTCAGGACAGC |
| | | GGCCCGCTGCTCATAAGACTCGGCCTTAGAACCCCAGT |
| | | ATCAGCAGAAGGACATTTTAGGACGGGACTTGGGTGAC |
| | | TCTAGGGCACTGGTTTTCTTTCCAGAGAGCGGAACAGG |
| | | CGAGGAAAAGTAGTCCCTTCTCGGCGATTCTGCGGAGG |
| | | GATCTCCGTGGGGCGGTGAACGCCGATGATTATATAAG |
| | | GACGCGCCGGGTGTGGCACAGCTAGTTCCGTCGCAGCC |
| | | GGGATTTGGGTCGCGGTTCTTGTTTGTGGATCGCTGTG |
| | | ATCGTCACTTGGTGAGTTGCGGGCTGCTGGGCTGGCCG |
| | | GGGCTTTCGTGGCCGCCGGGCCGCTCGGTGGGACGGAA |
| | | GCGTGTGGAGAGACCGCCAAGGGCTGTAGTCTGGGTCC |
| | | GCGAGCAAGGTTGCCCTGAACTGGGGGTTGGGGGGAGC |
| | | GCACAAAATGGCGGCTGTTCCCGAGTCTTGAATGGAAG |
| | | ACGCTTGTAAGGCGGGCTGTGAGGTCGTTGAAACAAGG |
| | | TGGGGGGCATGGTGGGCGGCAAGAACCCAAGGTCTTGA |
| | | GGCCTTCGCTAATGCGGGAAAGCTCTTATTCGGGTGAG |
| | | ATGGGCTGGGGCACCATCTGGGGACCCTGACGTGAAGT |
| | | TTGTCACTGACTGGAGAACTCGGGTTTGTCGTCTGGTT |
| | | GCGGGGGCGGCAGTTATGCGGTGCCGTTGGGCAGTGCA |
| | | CCCGTACCTTTGGGAGCGCGCGCCTCGTCGTGTCGTGA |
| | | CGTCACCCGTTCTGTTGGCTTATAATGCAGGGTGGGGC |
| | | CACCTGCCGGTAGGTGTGCGGTAGGCTTTTCTCCGTCG |
| | | CAGGACGCAGGGTTCGGGCCTAGGGTAGGCTCTCCTGA |
| | | ATCGACAGGCGCCGGACCTCTGGTGAGGGGAGGGATAA |
| | | GTGAGGCGTCAGTTTCTTTGGTCGGTTTTATGTACCTA |
| | | TCTTCTTAAGTAGCTGAAGCTCCGGTTTTGAACTATGC |
| | | GCTCGGGGTTGGCGAGTGTGTTTTGTGAAGTTTTTTAG |
| | | GCACCTTTTGAAATGTAATCATTTGGGTCAATATGTAA |
| | | TTTTCAGTGTTAGACTAGTAAA |
| 40 | Poly A; SV40 | GTTTATTGCAGCTTATAATGGTTACAAATAAAGCAATA |
| | | GCATCACAAATTTCACAAATAAAGCATTTTTTTCACTG |
| | | CATTCTAGTTGTGGTTTGTCCAAACTCATCAATGTATC |
| | | TTATCA |
| 41 | Poly A; bGH | GACTGTGCCTTCTAGTTGCCAGCCATCTGTTGTTTGCC |
| | | CCTCCCCCGTGCCTTCCTTGACCCTGGAAGGTGCCACT |
| | | CCCACTGTCCTTTCCTAATAAAATGAGGAAATTGCATC |
| | | GCATTGTCTGAGTAGGTGTCATTCTATTCTGGGGGGTG |
| | | GGGTGGGGCAGGACAGCAAGGGGGAGGATTGGGAAGAC |
| | | AATAGCAGGCATGCTGGGGATGCGGTGGGCTCTATGG |
| 42 | Envelope; | ATGAAACTCCCAACAGGAATGGTCATTTTATGTAGCCT |
| | RD114 | AATAATAGTTCGGGCAGGGTTTGACGACCCCCGCAAGG |
| | | CTATCGCATTAGTACAAAAACAACATGGTAAACCATGC |
| | | GAATGCAGCGGAGGGCAGGTATCCGAGGCCCCACCGAA |
| | | CTCCATCCAACAGGTAACTTGCCCAGGCAAGACGGCCT |
| | | ACTTAATGACCAACCAAAAATGGAAATGCAGAGTCACT |
| | | CCAAAAAATCTCACCCCTAGCGGGGGAGAACTCCAGAA |
| | | CTGCCCCTGTAACACTTTCCAGGACTCGATGCACAGTT |
| | | CTTGTTATACTGAATACCGGCAATGCAGGGCGAATAAT |
| | | AAGACATACTACACGGCCACCTTGCTTAAAATACGGTC |
| | | TGGGAGCCTCAACGAGGTACAGATATTACAAAACCCCA |
| | | ATCAGCTCCTACAGTCCCCTTGTAGGGGCTCTATAAAT |
| | | CAGCCCGTTTGCTGGAGTGCCACAGCCCCCATCCATAT |
| | | CTCCGATGGTGGAGGACCCCTCGATACTAAGAGAGTGT |
| | | GGACAGTCCAAAAAAGGCTAGAACAAATTCATAAGGCT |
| | | ATGCATCCTGAACTTCAATACCACCCCTTAGCCCTGCC |
| | | CAAAGTCAGAGATGACCTTAGCCTTGATGCACGGACTT |
| | | TTGATATCCTGAATACCACTTTTAGGTTACTCCAGATG |
| | | TCCAATTTTAGCCTTGCCCAAGATTGTTGGCTCTGTTT |
| | | AAAACTAGGTACCCCTACCCCTCTTGCGATACCCACTC |
| | | CCTCTTTAACCTACTCCCTAGCAGACTCCCTAGCGAAT |
| | | GCCTCCTGTCAGATTATACCTCCCCTCTTGGTTCAACC |
| | | GATGCAGTTCTCCAACTCGTCCTGTTTATCTTCCCCTT |
| | | TCATTAACGATACGGAACAAATAGACTTAGGTGCAGTC |
| | | ACCTTTACTAACTGCACCTCTGTAGCCAATGTCAGTAG |
| | | TCCTTTATGTGCCCTAAACGGGTCAGTCTTCCTCTGTG |
| | | GAAATAACATGGCATACACCTATTTACCCCAAAACTGG |
| | | ACAGGACTTTGCGTCCAAGCCTCCCTCCTCCCCGACAT |
| | | TGACATCATCCCGGGGGATGAGCCAGTCCCCATTCCTG |
| | | CCATTGATCATTATATACATAGACCTAAACGAGCTGTA |
| | | CAGTTCATCCCTTTACTAGCTGGACTGGGAATCACCGC |
| | | AGCATTCACCACCGGAGCTACAGGCCTAGGTGTCTCCG |
| | | TCACCCAGTATACAAAATTATCCCATCAGTTAATATCT |
| | | GATGTCCAAGTCTTATCCGGTACCATACAAGATTTACA |
| | | AGACCAGGTAGACTCGTTAGCTGAAGTAGTTCTCCAAA |
| | | ATAGGAGGGGACTGGACCTACTAACGGCAGAACAAGGA |
| | | GGAATTTGTTTAGCCTTACAAGAAAAATGCTGTTTTTA |
| | | TGCTAACAAGTCAGGAATTGTGAGAAACAAAATAAGAA |
| | | CCCTACAAGAAGAATTACAAAAACGCAGGGAAAGCCTG |
| | | GCATCCAACCCTCTCTGGACCGGGCTGCAGGGCTTTCT |
| | | TCCGTACCTCCTACCTCTCCTGGGACCCCTACTCACCC |
| | | TCCTACTCATACTAACCATTGGGCCATGCGTTTTCAAT |
| | | CGATTGGTCCAATTTGTTAAAGACAGGATCTCAGTGGT |
| | | CCAGGCTCTGGTTTTGACTCAGCAATATCACCAGCTAA |
| | | AACCCATAGAGTACGAGCCATGA |
| 43 | Envelope; | ATGCTTCTCACCTCAAGCCCGCACCACCTTCGGCACCA |
| | GALV | GATGAGTCCTGGGAGCTGGAAAAGACTGATCATCCTCT |
| | | TAAGCTGCGTATTCGGAGACGGCAAAACGAGTCTGCAG |
| | | AATAAGAACCCCCACCAGCCTGTGACCCTCACCTGGCA |
| | | GGTACTGTCCCAAACTGGGGACGTTGTCTGGGACAAAA |
| | | AGGCAGTCCAGCCCCTTTGGACTTGGTGGCCCTCTCTT |
| | | ACACCTGATGTATGTGCCCTGGCGGCCGGTCTTGAGTC |
| | | CTGGGATATCCCGGGATCCGATGTATCGTCCTCTAAAA |
| | | GAGTTAGACCTCCTGATTCAGACTATACTGCCGCTTAT |
| | | AAGCAAATCACCTGGGGAGCCATAGGGTGCAGCTACCC |
| | | TCGGGCTAGGACCAGGATGGCAAATTCCCCCTTCTACG |
| | | TGTGTCCCCGAGCTGGCCGAACCCATTCAGAAGCTAGG |
| | | AGGTGTGGGGGGCTAGAATCCCTATACTGTAAAGAATG |
| | | GAGTTGTGAGACCACGGGTACCGTTTATTGGCAACCCA |
| | | AGTCCTCATGGGACCTCATAACTGTAAAATGGGACCAA |
| | | AATGTGAAATGGGAGCAAAAATTTCAAAAGTGTGAACA |
| | | AACCGGCTGGTGTAACCCCCTCAAGATAGACTTCACAG |
| | | AAAAAGGAAAACTCTCCAGAGATTGGATAACGGAAAAA |
| | | ACCTGGGAATTAAGGTTCTATGTATATGGACACCCAGG |
| | | CATACAGTTGACTATCCGCTTAGAGGTCACTAACATGC |
| | | CGGTTGTGGCAGTGGGCCCAGACCCTGTCCTTGCGGAA |
| | | CAGGGACCTCCTAGCAAGCCCCTCACTCTCCCTCTCTC |
| | | CCCACGGAAAGCGCCGCCCACCCCTCTACCCCCGGCGG |
| | | CTAGTGAGCAAACCCCTGCGGTGCATGGAGAAACTGTT |
| | | ACCCTAAACTCTCCGCCTCCCACCAGTGGCGACCGACT |
| | | CTTTGGCCTTGTGCAGGGGGCCTTCCTAACCTTGAATG |
| | | CTACCAACCCAGGGGCCACTAAGTCTTGCTGGCTCTGT |
| | | TTGGGCATGAGCCCCCCTTATTATGAAGGGATAGCCTC |
| | | TTCAGGAGAGGTCGCTTATACCTCCAACCATACCCGAT |
| | | GCCACTGGGGGGCCCAAGGAAAGCTTACCCTCACTGAG |
| | | GTCTCCGGACTCGGGTCATGCATAGGGAAGGTGCCTCT |
| | | TACCCATCAACATCTTTGCAACCAGACCTTACCCATCA |
| | | ATTCCTCTAAAAACCATCAGTATCTGCTCCCCTCAAAC |
| | | CATAGCTGGTGGGCCTGCAGCACTGGCCTCACCCCCTG |
| | | CCTCTCCACCTCAGTTTTTAATCAGTCTAAAGACTTCT |
| | | GTGTCCAGGTCCAGCTGATCCCCCGCATCTATTACCAT |
| | | TCTGAAGAAACCTTGTTACAAGCCTATGACAAATCACC |
| | | CCCCAGGTTTAAAAGAGAGCCTGCCTCACTTACCCTAG |
| | | CTGTCTTCCTGGGGTTAGGGATTGCGGCAGGTATAGGT |
| | | ACTGGCTCAACCGCCCTAATTAAAGGGCCCATAGACCT |
| | | CCAGCAAGGCCTAACCAGCCTCCAAATCGCCATTGACG |
| | | CTGACCTCCGGGCCCTTCAGGACTCAATCAGCAAGCTA |
| | | GAGGACTCACTGACTTCCCTATCTGAGGTAGTACTCCA |
| | | AAATAGGAGAGGCCTTGACTTACTATTCCTTAAAGAAG |
| | | GAGGCCTCTGCGCGGCCCTAAAAGAAGAGTGCTGTTTT |
| | | TATGTAGACCACTCAGGTGCAGTACGAGACTCCATGAA |
| | | AAAACTTAAAGAAAGACTAGATAAAAGACAGTTAGAGC |
| | | GCCAGAAAAACCAAAACTGGTATGAAGGGTGGTTCAAT |
| | | AACTCCCCTTGGTTTACTACCCTACTATCAACCATCGC |
| | | TGGGCCCCTATTGCTCCTCCTTTTGTTACTCACTCTTG |
| | | GGCCCTGCATCATCAATAAATTAATCCAATTCATCAAT |
| | | GATAGGATAAGTGCAGTCAAAATTTTAGTCCTTAGACA |
| | | GAAATATCAGACCCTAGATAACGAGGAAAACCTTTAA |
| 44 | Envelope; | ATGGTTCCGCAGGTTCTTTTGTTTGTACTCCTTCTGGG |
| | FUG | TTTTTCGTTGTGTTTCGGGAAGTTCCCCATTTACACGA |
| | | TACCAGACGAACTTGGTCCCTGGAGCCCTATTGACATA |
| | | CACCATCTCAGCTGTCCAAATAACCTGGTTGTGGAGGA |
| | | TGAAGGATGTACCAACCTGTCCGAGTTCTCCTACATGG |
| | | AACTCAAAGTGGGATACATCTCAGCCATCAAAGTGAAC |
| | | GGGTTCACTTGCACAGGTGTTGTGACAGAGGCAGAGAC |
| | | CTACACCAACTTTGTTGGTTATGTCACAACCACATTCA |
| | | AGAGAAAGCATTTCCGCCCCACCCCAGACGCATGTAGA |
| | | GCCGCGTATAACTGGAAGATGGCCGGTGACCCCAGATA |
| | | TGAAGAGTCCCTACACAATCCATACCCCGACTACCACT |
| | | GGCTTCGAACTGTAAGAACCACCAAAGAGTCCCTCATT |
| | | ATCATATCCCCAAGTGTGACAGATTTGGACCCATATGA |
| | | CAAATCCCTTCACTCAAGGGTCTTCCCTGGCGGAAAGT |
| | | GCTCAGGAATAACGGTGTCCTCTACCTACTGCTCAACT |
| | | AACCATGATTACACCATTTGGATGCCCGAGAATCCGAG |
| | | ACCAAGGACACCTTGTGACATTTTTACCAATAGCAGAG |
| | | GGAAGAGAGCATCCAACGGGAACAAGACTTGCGGCTTT |
| | | GTGGATGAAAGAGGCCTGTATAAGTCTCTAAAAGGAGC |
| | | ATGCAGGCTCAAGTTATGTGGAGTTCTTGGACTTAGAC |
| | | TTATGGATGGAACATGGGTCGCGATGCAAACATCAGAT |
| | | GAGACCAAATGGTGCCCTCCAGATCAGTTGGTGAATTT |
| | | GCACGACTTTCGCTCAGACGAGATCGAGCATCTCGTTG |
| | | TGGAGGAGTTAGTTAAGAAAAGAGAGGAATGTCTGGAT |
| | | GCATTAGAGTCCATCATGACCACCAAGTCAGTAAGTTT |
| | | CAGACGTCTCAGTCACCTGAGAAAACTTGTCCCAGGGT |
| | | TTGGAAAAGCATATACCATATTCAACAAAACCTTGATG |
| | | GAGGCTGATGCTCACTACAAGTCAGTCCGGACCTGGAA |
| | | TGAGATCATCCCCTCAAAAGGGTGTTTGAAAGTTGGAG |
| | | GAAGGTGCCATCCTCATGTGAACGGGGTGTTTTTCAAT |
| | | GGTATAATATTAGGGCCTGACGACCATGTCCTAATCCC |
| | | AGAGATGCAATCATCCCTCCTCCAGCAACATATGGAGT |
| | | TGTTGGAATCTTCAGTTATCCCCCTGATGCACCCCCTG |
| | | GCAGACCCTTCTACAGTTTTCAAAGAAGGTGATGAGGC |
| | | TGAGGATTTTGTTGAAGTTCACCTCCCCGATGTGTACA |
| | | AACAGATCTCAGGGGTTGACCTGGGTCTCCCGAACTGG |
| | | GGAAAGTATGTATTGATGACTGCAGGGGCCATGATTGG |
| | | CCTGGTGTTGATATTTTCCCTAATGACATGGTGCAGAG |
| | | TTGGTATCCATCTTTGCATTAAATTAAAGCACACCAAG |
| | | AAAAGACAGATTTATACAGACATAGAGATGAACCGACT |
| | | TGGAAAGTAA |
| 45 | Envelope; | ATGGGTCAGATTGTGACAATGTTTGAGGCTCTGCCTCA |
| | LCMV | CATCATCGATGAGGTGATCAACATTGTCATTATTGTGC |
| | | TTATCGTGATCACGGGTATCAAGGCTGTCTACAATTTT |
| | | GCCACCTGTGGGATATTCGCATTGATCAGTTTCCTACT |
| | | TCTGGCTGGCAGGTCCTGTGGCATGTACGGTCTTAAGG |
| | | GACCCGACATTTACAAAGGAGTTTACCAATTTAAGTCA |
| | | GTGGAGTTTGATATGTCACATCTGAACCTGACCATGCC |
| | | CAACGCATGTTCAGCCAACAACTCCCACCATTACATCA |
| | | GTATGGGGACTTCTGGACTAGAATTGACCTTCACCAAT |
| | | GATTCCATCATCAGTCACAACTTTTGCAATCTGACCTC |
| | | TGCCTTCAACAAAAAGACCTTTGACCACACACTCATGA |
| | | GTATAGTTTCGAGCCTACACCTCAGTATCAGAGGGAAC |
| | | TCCAACTATAAGGCAGTATCCTGCGACTTCAACAATGG |
| | | CATAACCATCCAATACAACTTGACATTCTCAGATCGAC |
| | | AAAGTGCTCAGAGCCAGTGTAGAACCTTCAGAGGTAGA |
| | | GTCCTAGATATGTTTAGAACTGCCTTCGGGGGGAAATA |
| | | CATGAGGAGTGGCTGGGGCTGGACAGGCTCAGATGGCA |
| | | AGACCACCTGGTGTAGCCAGACGAGTTACCAATACCTG |
| | | ATTATACAAAATAGAACCTGGGAAAACCACTGCACATA |
| | | TGCAGGTCCTTTTGGGATGTCCAGGATTCTCCTTTCCC |
| | | AAGAGAAGACTAAGTTCTTCACTAGGAGACTAGCGGGC |
| | | ACATTCACCTGGACTTTGTCAGACTCTTCAGGGGTGGA |
| | | GAATCCAGGTGGTTATTGCCTGACCAAATGGATGATTC |
| | | TTGCTGCAGAGCTTAAGTGTTTCGGGAACACAGCAGTT |
| | | GCGAAATGCAATGTAAATCATGATGCCGAATTCTGTGA |
| | | CATGCTGCGACTAATTGACTACAACAAGGCTGCTTTGA |
| | | GTAAGTTCAAAGAGGACGTAGAATCTGCCTTGCACTTA |
| | | TTCAAAACAACAGTGAATTCTTTGATTTCAGATCAACT |
| | | ACTGATGAGGAACCACTTGAGAGATCTGATGGGGGTGC |
| | | CATATTGCAATTACTCAAAGTTTTGGTACCTAGAACAT |
| | | GCAAAGACCGGCGAAACTAGTGTCCCCAAGTGCTGGCT |
| | | TGTCACCAATGGTTCTTACTTAAATGAGACCCACTTCA |
| | | GTGATCAAATCGAACAGGAAGCCGATAACATGATTACA |
| | | GAGATGTTGAGGAAGGATTACATAAAGAGGCAGGGGAG |
| | | TACCCCCCTAGCATTGATGGACCTTCTGATGTTTTCCA |
| | | CATCTGCATATCTAGTCAGCATCTTCCTGCACCTTGTC |
| | | AAAATACCAACACACAGGCACATAAAAGGTGGCTCATG |
| | | TCCAAAGCCACACCGATTAACCAACAAAGGAATTTGTA |
| | | GTTGTGGTGCATTTAAGGTGCCTGGTGTAAAAACCGTC |
| | | TGGAAAAGACGCTGA |
| 46 | Envelope; FPV | ATGAACACTCAAATCCTGGTTTTCGCCCTTGTGGCAGT |
| | | CATCCCCACAAATGCAGACAAAATTTGTCTTGGACATC |
| | | ATGCTGTATCAAATGGCACCAAAGTAAACACACTCACT |
| | | GAGAGAGGAGTAGAAGTTGTCAATGCAACGGAAACAGT |
| | | GGAGCGGACAAACATCCCCAAAATTTGCTCAAAAGGGA |
| | | AAAGAACCACTGATCTTGGCCAATGCGGACTGTTAGGG |
| | | ACCATTACCGGACCACCTCAATGCGACCAATTTCTAGA |
| | | ATTTTCAGCTGATCTAATAATCGAGAGACGAGAAGGAA |
| | | ATGATGTTTGTTACCCGGGGAAGTTTGTTAATGAAGAG |
| | | GCATTGCGACAAATCCTCAGAGGATCAGGTGGGATTGA |
| | | CAAAGAAACAATGGGATTCACATATAGTGGAATAAGGA |
| | | CCAACGGAACAACTAGTGCATGTAGAAGATCAGGGTCT |
| | | TCATTCTATGCAGAAATGGAGTGGCTCCTGTCAAATAC |
| | | AGACAATGCTGCTTTCCCACAAATGACAAAATCATACA |
| | | AAAACACAAGGAGAGAATCAGCTCTGATAGTCTGGGGA |
| | | ATCCACCATTCAGGATCAACCACCGAACAGACCAAACT |
| | | ATATGGGAGTGGAAATAAACTGATAACAGTCGGGAGTT |
| | | CCAAATATCATCAATCTTTTGTGCCGAGTCCAGGAACA |
| | | CGACCGCAGATAAATGGCCAGTCCGGACGGATTGATTT |
| | | TCATTGGTTGATCTTGGATCCCAATGATACAGTTACTT |
| | | TTAGTTTCAATGGGGCTTTCATAGCTCCAAATCGTGCC |
| | | AGCTTCTTGAGGGGAAAGTCCATGGGGATCCAGAGCGA |
| | | TGTGCAGGTTGATGCCAATTGCGAAGGGGAATGCTACC |
| | | ACAGTGGAGGGACTATAACAAGCAGATTGCCTTTTCAA |
| | | AACATCAATAGCAGAGCAGTTGGCAAATGCCCAAGATA |
| | | TGTAAAACAGGAAAGTTTATTATTGGCAACTGGGATGA |
| | | AGAACGTTCCCGAACCTTCCAAAAAAAGGAAAAAAAGA |
| | | GGCCTGTTTGGCGCTATAGCAGGGTTTATTGAAAATGG |
| | | TTGGGAAGGTCTGGTCGACGGGTGGTACGGTTTCAGGC |
| | | ATCAGAATGCACAAGGAGAAGGAACTGCAGCAGACTAC |
| | | AAAAGCACCCAATCGGCAATTGATCAGATAACCGGAAA |
| | | GTTAAATAGACTCATTGAGAAAACCAACCAGCAATTTG |
| | | AGCTAATAGATAATGAATTCACTGAGGTGGAAAAGCAG |
| | | ATTGGCAATTTAATTAACTGGACCAAAGACTCCATCAC |
| | | AGAAGTATGGTCTTACAATGCTGAACTTCTTGTGGCAA |
| | | TGGAAAACCAGCACACTATTGATTTGGCTGATTCAGAG |
| | | ATGAACAAGCTGTATGAGCGAGTGAGGAAACAATTAAG |
| | | GGAAAATGCTGAAGAGGATGGCACTGGTTGCTTTGAAA |
| | | TTTTTCATAAATGTGACGATGATTGTATGGCTAGTATA |
| | | AGGAACAATACTTATGATCACAGCAAATACAGAGAAGA |
| | | AGCGATGCAAAATAGAATACAAATTGACCCAGTCAAAT |
| | | TGAGTAGTGGCTACAAAGATGTGATACTTTGGTTTAGC |
| | | TTCGGGGCATCATGCTTTTTGCTTCTTGCCATTGCAAT |
| | | GGGCCTTGTTTTCATATGTGTGAAGAACGGAAACATGC |
| | | GGTGCACTATTTGTATATAA |
| 47 | Envelope; | AGTGTAACAGAGCACTTTAATGTGTATAAGGCTACTAG |
| | RRV | ACCATACCTAGCACATTGCGCCGATTGCGGGGACGGGT |
| | | ACTTCTGCTATAGCCCAGTTGCTATCGAGGAGATCCGA |
| | | GATGAGGCGTCTGATGGCATGCTTAAGATCCAAGTCTC |
| | | CGCCCAAATAGGTCTGGACAAGGCAGGCACCCACGCCC |
| | | ACACGAAGCTCCGATATATGGCTGGTCATGATGTTCAG |
| | | GAATCTAAGAGAGATTCCTTGAGGGTGTACACGTCCGC |
| | | AGCGTGCTCCATACATGGGACGATGGGACACTTCATCG |
| | | TCGCACACTGTCCACCAGGCGACTACCTCAAGGTTTCG |
| | | TTCGAGGACGCAGATTCGCACGTGAAGGCATGTAAGGT |
| | | CCAATACAAGCACAATCCATTGCCGGTGGGTAGAGAGA |
| | | AGTTCGTGGTTAGACCACACTTTGGCGTAGAGCTGCCA |
| | | TGCACCTCATACCAGCTGACAACGGCTCCCACCGACGA |
| | | GGAGATTGACATGCATACACCGCCAGATATACCGGATC |
| | | GCACCCTGCTATCACAGACGGCGGGCAACGTCAAAATA |
| | | ACAGCAGGCGGCAGGACTATCAGGTACAACTGTACCTG |
| | | CGGCCGTGACAACGTAGGCACTACCAGTACTGACAAGA |
| | | CCATCAACACATGCAAGATTGACCAATGCCATGCTGCC |
| | | GTCACCAGCCATGACAAATGGCAATTTACCTCTCCATT |
| | | TGTTCCCAGGGCTGATCAGACAGCTAGGAAAGGCAAGG |
| | | TACACGTTCCGTTCCCTCTGACTAACGTCACCTGCCGA |
| | | GTGCCGTTGGCTCGAGCGCCGGATGCCACCTATGGTAA |
| | | GAAGGAGGTGACCCTGAGATTACACCCAGATCATCCGA |
| | | CGCTCTTCTCCTATAGGAGTTTAGGAGCCGAACCGCAC |
| | | CCGTACGAGGAATGGGTTGACAAGTTCTCTGAGCGCAT |
| | | CATCCCAGTGACGGAAGAAGGGATTGAGTACCAGTGGG |
| | | GCAACAACCCGCCGGTCTGCCTGTGGGCGCAACTGACG |
| | | ACCGAGGGCAAACCCCATGGCTGGCCACATGAAATCAT |
| | | TCAGTACTATTATGGACTATACCCCGCCGCCACTATTG |
| | | CCGCAGTATCCGGGGCGAGTCTGATGGCCCTCCTAACT |
| | | CTGGCGGCCACATGCTGCATGCTGGCCACCGCGAGGAG |
| | | AAAGTGCCTAACACCGTACGCCCTGACGCCAGGAGCGG |
| | | TGGTACCGTTGACACTGGGGCTGCTTTGCTGCGCACCG |
| | | AGGGCGAATGCA |
| 48 | Envelope; | ATGGGTGTTACAGGAATATTGCAGTTACCTCGTGATCG |
| | Ebola | ATTCAAGAGGACATCATTCTTTCTTTGGGTAATTATCC |
| | | TTTTCCAAAGAACATTTTCCATCCCACTTGGAGTCATC |
| | | CACAATAGCACATTACAGGTTAGTGATGTCGACAAACT |
| | | GGTTTGCCGTGACAAACTGTCATCCACAAATCAATTGA |
| | | GATCAGTTGGACTGAATCTCGAAGGGAATGGAGTGGCA |
| | | ACTGACGTGCCATCTGCAACTAAAAGATGGGGCTTCAG |
| | | GTCCGGTGTCCCACCAAAGGTGGTCAATTATGAAGCTG |
| | | GTGAATGGGCTGAAAACTGCTACAATCTTGAAATCAAA |
| | | AAACCTGACGGGAGTGAGTGTCTACCAGCAGCGCCAGA |
| | | CGGGATTCGGGGCTTCCCCCGGTGCCGGTATGTGCACA |
| | | AAGTATCAGGAACGGGACCGTGTGCCGGAGACTTTGCC |
| | | TTCCACAAAGAGGGTGCTTTCTTCCTGTATGACCGACT |
| | | TGCTTCCACAGTTATCTACCGAGGAACGACTTTCGCTG |
| | | AAGGTGTCGTTGCATTTCTGATACTGCCCCAAGCTAAG |
| | | AAGGACTTCTTCAGCTCACACCCCTTGAGAGAGCCGGT |
| | | CAATGCAACGGAGGACCCGTCTAGTGGCTACTATTCTA |
| | | CCACAATTAGATATCAAGCTACCGGTTTTGGAACCAAT |
| | | GAGACAGAGTATTTGTTCGAGGTTGACAATTTGACCTA |
| | | CGTCCAACTTGAATCAAGATTCACACCACAGTTTCTGC |
| | | TCCAGCTGAATGAGACAATATATACAAGTGGGAAAAGG |
| | | AGCAATACCACGGGAAAACTAATTTGGAAGGTCAACCC |
| | | CGAAATTGATACAACAATCGGGGAGTGGGCCTTCTGGG |
| | | AAACTAAAAAAACCTCACTAGAAAAATTCGCAGTGAAG |
| | | AGTTGTCTTTCACAGCTGTATCAAACAGAGCCAAAAAC |
| | | ATCAGTGGTCAGAGTCCGGCGCGAACTTCTTCCGACCC |
| | | AGGGACCAACACAACAACTGAAGACCACAAAATCATGG |
| | | CTTCAGAAAATTCCTCTGCAATGGTTCAAGTGCACAGT |
| | | CAAGGAAGGGAAGCTGCAGTGTCGCATCTGACAACCCT |
| | | TGCCACAATCTCCACGAGTCCTCAACCCCCCACAACCA |
| | | AACCAGGTCCGGACAACAGCACCCACAATACACCCGTG |
| | | TATAAACTTGACATCTCTGAGGCAACTCAAGTTGAACA |
| | | ACATCACCGCAGAACAGACAACGACAGCACAGCCTCCG |
| | | ACACTCCCCCCGCCACGACCGCAGCCGGACCCCTAAAA |
| | | GCAGAGAACACCAACACGAGCAAGGGTACCGACCTCCT |
| | | GGACCCCGCCACCACAACAAGTCCCCAAAACCACAGCG |
| | | AGACCGCTGGCAACAACAACACTCATCACCAAGATACC |
| | | GGAGAAGAGAGTGCCAGCAGCGGGAAGCTAGGCTTAAT |
| | | TACCAATACTATTGCTGGAGTCGCAGGACTGATCACAG |
| | | GCGGGAGGAGAGCTCGAAGAGAAGCAATTGTCAATGCT |
| | | CAACCCAAATGCAACCCTAATTTACATTACTGGACTAC |
| | | TCAGGATGAAGGTGCTGCAATCGGACTGGCCTGGATAC |
| | | CATATTTCGGGCCAGCAGCCGAGGGAATTTACATAGAG |
| | | GGGCTGATGCACAATCAAGATGGTTTAATCTGTGGGTT |
| | | GAGACAGCTGGCCAACGAGACGACTCAAGCTCTTCAAC |
| | | TGTTCCTGAGAGCCACAACCGAGCTACGCACCTTTTCA |
| | | ATCCTCAACCGTAAGGCAATTGATTTCTTGCTGCAGCG |
| | | ATGGGGCGGCACATGCCACATTTTGGGACCGGACTGCT |
| | | GTATCGAACCACATGATTGGACCAAGAACATAACAGAC |
| | | AAAATTGATCAGATTATTCATGATTTTGTTGATAAAAC |
| | | CCTTCCGGACCAGGGGGACAATGACAATTGGTGGACAG |
| | | GATGGAGACAATGGATACCGGCAGGTATTGGAGTTACA |
| | | GGCGTTATAATTGCAGTTATCGCTTTATTCTGTATATG |
| | | CAAATTTGTCTTTTAG |
| 49 | FDPS target | GTCCTGGAGTACAATGCCATT |
| | sequence #1 | |
| 50 | FDPS target | GCAGGATTTCGTTCAGCACTT |
| | sequence #2 | |
| 51 | FDPS target | GCCATGTACATGGCAGGAATT |
| | sequence #3 | |
| 52 | FDPS target | GCAGAAGGAGGCTGAGAAAGT |
| | sequence #4 | |
| 53 | miR30 FDPS | AAGGTATATTGCTGTTGACAGTGAGCGACACTTTCTCA |
| | sequence #1 | GCCTCCTTCTGCGTGAAGCCACAGATGGCAGAAGGAGG |
| | | CTGAGAAAGTGCTGCCTACTGCCTCGGACTTCAAGGGG |
| | | CT |
| 54 | miR30 FDPS | AAGGTATATTGCTGTTGACAGTGAGCGACACTTTCTCA |
| | sequence #2 | GCCTCCTTCTGCGTGAAGCCACAGATGGCAGAAGGGCT |
| | | GAGAAAGTGCTGCCTACTGCCTCGGACTTCAAGGGGCT |
| 55 | miR30 FDPS | TGCTGTTGACAGTGAGCGACTTTCTCAGCCTCCTTCTG |
| | sequence #3 | CGTGAAGCCACAGATGGCAGAAGGAGGCTGAGAAAGTT |
| | | GCCTACTGCCTCGGA |
| 56 | miR155 FDPS | CCTGGAGGCTTGCTGAAGGCTGTATGCTGACTTTCTCA |
| | sequence #1 | GCCTCCTTCTGCTTTTGGCCACTGACTGAGCAGAAGGG |
| | | CTGAGAAAGTCAGGACACAAGGCCTGTTACTAGCACTC |
| | | A |
| 57 | miR21 FDPS | CATCTCCATGGCTGTACCACCTTGTCGGGACTTTCTCA |
| | sequence #1 | GCCTCCTTCTGCCTGTTGAATCTCATGGCAGAAGGAGG |
| | | CGAGAAAGTCTGACATTTTGGTATCTTTCATCTGACCA |
| 58 | miR185 FDPS | GGGCCTGGCTCGAGCAGGGGGCGAGGGATACTTTCTCA |
| | sequence #1 | GCCTCCTTCTGCTGGTCCCCTCCCCGCAGAAGGAGGCT |
| | | GAGAAAGTCCTTCCCTCCCAATGACCGCGTCTTCGTCG |
| 59 | Forward | AGGAATTGATGGCGAGAAGG |
| | primer | |
| 60 | Reverse primer | CCCAAAGAGGTCAAGGTAATCA |
| 61 | Forward | AGCGCGGCTACAGCTTCA |
| | primer | |
| 62 | Reverse primer | GGCGACGTAGCACAGCTTCT |
| 63 | Forward | CACTGTCGTCATTCCATGCT |
| | primer | |
| 64 | Reverse primer | GCCTCTTGACATTCTCCTC |
| 65 | Reverse primer | AAAGTCAGTGGGGACAGTGG |
| 66 | miR155 CD47 | CCTGGAGGCTTGCTGAAGGCTGTATGCTGTTAGCTCGA |
| | target sequence | TGATCGTTTCACGTTTTGGCCACTGACTGACGTGAAAC |
| | #2 | GCATCGAGCTAACAGGACACAAGGCCTGTTACTAGCAC |
| | | TCA |
| 67 | miR155 CD47 | CCTGGAGGCTTGCTGAAGGCTGTATGCTGAAGAATGGC |
| | target sequence | TCCAACAATGACGTTTTGGCCACTGACTGACGTCATTG |
| | #3 | TGAGCCATTCTTCAGGACACAAGGCCTGTTACTAGCAC |
| | | TCA |
| 68 | miR155 CD47 | CCTGGAGGCTTGCTGAAGGCTGTATGCTGTATACACGC |
| | target sequence | CGCAATACAGAGGTTTTGGCCACTGACTGACCTCTGTA |
| | #4 | TCGGCGTGTATACAGGACACAAGGCCTGTTACTAGCAC |
| | | TCA |
| 69 | Forward | GGACTATCCTGCTGCCAA |
| | primer | |
| 70 | miR155 cMyc | CCTGGAGGCTTGCTGAAGGCTGTATGCTGTGTTCGCCT |
| | sequence | CTTGACATTCTCTTTTGGCCACTGACTGAGAGAATGTA |
| | | GAGGCGAACACAGGACACAAGGCCTGTTACTAGCACTC |
| | | A |
| 71 | cMyc target | GAGAATGTCAAGAGGCGAACA |
| | sequence | |
| 72 | CMV promoter | ATTATGCCCAGTACATGACCTTATGGGACTTTCCTACT |
| | sequence | TGGCAGTACATCTACGTATTAGTCATCGCTATTACCAT |
| | | GGTGATGCGGTTTTGGCAGTACATCAATGGGCGTGGAT |
| | | AGCGGTTTGACTCACGGGGATTTCCAAGTCTCCACCCC |
| | | ATTGACGTCAATGGGAGTTTGTTTTGGCACCAAAATCA |
| | | ACGGGACTTTCCAAAATGTCGTAACAACTCCGCCCCAT |
| | | TGACGCAAATGGGCGGTAGGCGTGTACGGTGGGAGGTT |
| | | TATATAAGCAGAGCTCGTTTAGTGAACCGTCAGATCGC |
| | | CTGGAGACGCCATCCACGCTGTTTT |
| 73 | GFP T2A | ATGCCCGCCATGAAGATCGAGTGCCGCATCACCGGCAC |
| | Luciferase | CCTGAACGGCGTGGAGTTCGAGCTGGTGGGCGGCGGAG |
| | sequence | AGGGCACCCCCGAGCAGGGCCGCATGACCAACAAGATG |
| | | AAGAGCACCAAAGGCGCCCTGACCTTCAGCCCCTACCT |
| | | GCTGAGCCACGTGATGGGCTACGGCTTCTACCACTTCG |
| | | GCACCTACCCCAGCGGCTACGAGAACCCCTTCCTGCAC |
| | | GCCATCAACAACGGCGGCTACACCAACACCCGCATCGA |
| | | GAAGTACGAGGACGGCGGCGTGCTGCACGTGAGCTTCA |
| | | GCTACCGCTACGAGGCCGGCCGCGTGATCGGCGACTTC |
| | | AAGGTGGTGGGCACCGGCTTCCCCGAGGACAGCGTGAT |
| | | CTTCACCGACAAGATCATCCGCAGCAACGCCACCGTGG |
| | | AGCACCTGCACCCCATGGGCGATAACGTGCTGGTGGGC |
| | | AGCTTCGCCCGCACCTTCAGCCTGCGCGACGGCGGCTA |
| | | CTACAGCTTCGTGGTGGACAGCCACATGCACTTCAAGA |
| | | GCGCCATCCACCCCAGCATCCTGCAGAACGGGGGCCCC |
| | | ATGTTCGCCTTCCGCCGCGTGGAGGAGCTGCACAGCAA |
| | | CACCGAGCTGGGCATCGTGGAGTACCAGCACGCCTTCA |
| | | AGACCCCCATCGCCTTCGCCAGATCTCGAGATATCAGC |
| | | CATGGCTTCCCGCCGGCGGTGGCGGCGCAGGATGATGG |
| | | CACGCTGCCCATGTCTTGTGCCCAGGAGAGCGGGATGG |
| | | ACCGTCACCCTGCAGCCTGTGCTTCTGCTAGGATCAAT |
| | | GTGACCGGTGAGGGCAGAGGAAGTCTTCTAACATGCGG |
| | | TGACGTGGAGGAGAATCCCGGCCCTTCCGGTATGGAAG |
| | | ACGCCAAAAACATAAAGAAAGGCCCGGCGCCATTCTAT |
| | | CCGCTAGAGGATGGAACCGCTGGAGAGCAACTGCATAA |
| | | GGCTATGAAGAGATACGCCCTGGTTCCTGGAACAATTG |
| | | CTTTTACAGATGCACATATCGAGGTGAACATCACGTAC |
| | | GCGGAATACTTCGAAATGTCCGTTCGGTTGGCAGAAGC |
| | | TATGAAACGATATGGGCTGAATACAAATCACAGAATCG |
| | | TCGTATGCAGTGAAAACTCTCTTCAATTCTTTATGCCG |
| | | GTGTTGGGCGCGTTATTTATCGGAGTTGCAGTTGCGCC |
| | | CGCGAACGACATTTATAATGAACGTGAATTGCTCAACA |
| | | GTATGAACATTTCGCAGCCTACCGTAGTGTTTGTTTCC |
| | | AAAAAGGGGTTGCAAAAAATTTTGAACGTGCAAAAAAA |
| | | ATTACCAATAATCCAGAAAATTATTATCATGGATTCTA |
| | | AAACGGATTACCAGGGATTTCAGTCGATGTACACGTTC |
| | | GTCACATCTCATCTACCTCCCGGTTTTAATGAATACGA |
| | | TTTTGTACCAGAGTCCTTTGATCGTGACAAAACAATTG |
| | | CACTGATAATGAACTCCTCTGGATCTACTGGGTTACCT |
| | | AAGGGTGTGGCCCTTCCGCATAGAACTGCCTGCGTCAG |
| | | ATTCTCGCATGCCAGAGATCCTATTTTTGGCAATCAAA |
| | | TCATTCCGGATACTGCGATTTTAAGTGTTGTTCCATTC |
| | | CATCACGGTTTTGGAATGTTTACTACACTCGGATATTT |
| | | GATATGTGGATTTCGAGTCGTCTTAATGTATAGATTTG |
| | | AAGAAGAGCTGTTTTTACGATCCCTTCAGGATTACAAA |
| | | ATTCAAAGTGCGTTGCTAGTACCAACCCTATTTTCATT |
| | | CTTCGCCAAAAGCACTCTGATTGACAAATACGATTTAT |
| | | CTAATTTACACGAAATTGCTTCTGGGGGCGCACCTCTT |
| | | TCGAAAGAAGTCGGGGAAGCGGTTGCAAAACGCTTCCA |
| | | TCTTCCAGGGATACGACAAGGATATGGGCTCACTGAGA |
| | | CTACATCAGCTATTCTGATTACACCCGAGGGGGATGAT |
| | | AAACCGGGCGCGGTCGGTAAAGTTGTTCCATTTTTTGA |
| | | AGCGAAGGTTGTGGATCTGGATACCGGGAAAACGCTGG |
| | | GCGTTAATCAGAGAGGCGAATTATGTGTCAGAGGACCT |
| | | ATGATTATGTCCGGTTATGTAAACAATCCGGAAGCGAC |
| | | CAACGCCTTGATTGACAAGGATGGATGGCTACATTCTG |
| | | GAGACATAGCTTACTGGGACGAAGACGAACACTTCTTC |
| | | ATAGTTGACCGCTTGAAGTCTTTAATTAAATACAAAGG |
| | | ATACCAGGTGGCCCCCGCTGAATTGGAGTCGATATTGT |
| | | TACAACACCCCAACATCTTCGACGCGGGCGTGGCAGGT |
| | | CTTCCCGACGATGACGCCGGTGAACTTCCCGCCGCCGT |
| | | TGTTGTTTTGGAGCACGGAAAGACGATGACGGAAAAAG |
| | | AGATCGTGGATTACGTCGCCAGTCAAGTAACAACCGCG |
| | | AAAAAGTTGCGCGGAGGAGTTGTGTTTGTGGACGAAGT |
| | | ACCGAAAGGTCTTACCGGAAAACTCGACGCAAGAAAAA |
| | | TCAGAGAGATCCTCATAAAGGCCAAGAAGGGCGGAAAG |
| | | TCCAAATTGTAA |
| 74 | Rous Sarcoma | GTAGTCTTATGCAATACTCTTGTAGTCTTGCAACATGG |
| | virus (RSV) | TAACGATGAGTTAGCAACATGCCTTACAAGGAGAGAAA |
| | promoter | AAGCACCGTGCATGCCGATTGGTGGAAGTAAGGTGGTA |
| | | CGATCGTGCCTTATTAGGAAGGCAACAGACGGGTCTGA |
| | | CATGGATTGGACGAACCACTGAATTGCCGCATTGCAGA |
| | | GATATTGTATTTAAGTGCCTAGCTCGATACAATAAACG |
| 75 | 5′ Long | GGTCTCTCTGGTTAGACCAGATCTGAGCCTGGGAGCTC |
| | terminal repeat | TCTGGCTAACTAGGGAACCCACTGCTTAAGCCTCAATA |
| | (LTR) | AAGCTTGCCTTGAGTGCTTCAAGTAGTGTGTGCCCGTC |
| | | TGTTGTGTGACTCTGGTAACTAGAGATCCCTCAGACCC |
| | | TTTTAGTCAGTGTGGAAAATCTCTAGCA |
| 76 | Psi Packaging | TACGCCAAAAATTTTGACTAGCGGAGGCTAGAAGGAGA |
| | signal | GAG |
| 77 | Rev response | AGGAGCTTTGTTCCTTGGGTTCTTGGGAGCAGCAGGAA |
| | element (RRE) | GCACTATGGGCGCAGCCTCAATGACGCTGACGGTACAG |
| | | GCCAGACAATTATTGTCTGGTATAGTGCAGCAGCAGAA |
| | | CAATTTGCTGAGGGCTATTGAGGCGCAACAGCATCTGT |
| | | TGCAACTCACAGTCTGGGGCATCAAGCAGCTCCAGGCA |
| | | AGAATCCTGGCTGTGGAAAGATACCTAAAGGATCAACA |
| | | GCTCC |
| 78 | Central | TTTTAAAAGAAAAGGGGGGATTGGGGGGTACAGTGCAG |
| | polypurine | GGGAAAGAATAGTAGACATAATAGCAACAGACATACAA |
| | tract (cPPT) | ACTAAAGAATTACAAAAACAAATTACAAAATTCAAAAT |
| | | TTTA |
| 79 | Long WPRE | AATCAACCTCTGATTACAAAATTTGTGAAAGATTGACT |
| | sequence | GGTATTCTTAACTATGTTGCTCCTTTTACGCTATGTGG |
| | | ATACGCTGCTTTAATGCCTTTGTATCATGCTATTGCTT |
| | | CCCGTATGGCTTTCATTTTCTCCTCCTTGTATAAATCC |
| | | TGGTTGCTGTCTCTTTATGAGGAGTTGTGGCCCGTTGT |
| | | CAGGCAACGTGGCGTGGTGTGCACTGTGTTTGCTGACG |
| | | CAACCCCCACTGGTTGGGGCATTGCCACCACCTGTCAG |
| | | CTCCTTTCCGGGACTTTCGCTTTCCCCCTCCCTATTGC |
| | | CACGGCGGAACTCATCGCCGCCTGCCTTGCCCGCTGCT |
| | | GGACAGGGGCTCGGCTGTTGGGCACTGACAATTCCGTG |
| | | GTGTTGTCGGGGAAATCATCGTCCTTTCCTTGGCTGCT |
| | | CGCCTGTGTTGCCACCTGGATTCTGCGCGGGACGTCCT |
| | | TCTGCTACGTCCCTTCGGCCCTCAATCCAGCGGACCTT |
| | | CCTTCCCGCGGCCTGCTGCCGGCTCTGCGGCCTCTTCC |
| | | GCGTCTTCGCCTTCGCCCTCAGACGAGTCGGATCTCCC |
| | | TTTGGGCCGCCTCCCCGCCT |
| 80 | 3′ delta LTR | TGGAAGGGCTAATTCACTCCCAACGAAGATAAGATCTG |
| | | CTTTTTGCTTGTACTGGGTCTCTCTGGTTAGACCAGAT |
| | | CTGAGCCTGGGAGCTCTCTGGCTAACTAGGGAACCCAC |
| | | TGCTTAAGCCTCAATAAAGCTTGCCTTGAGTGCTTCAA |
| | | GTAGTGTGTGCCCGTCTGTTGTGTGACTCTGGTAACTA |
| | | GAGATCCCTCAGACCCTTTTAGTCAGTGTGGAAAATCT |
| | | CTAGCAGTAGTAGTTCATGTCA |
| 81 | Envelope; | ATGGAAGGTCCAGCGTTCTCAAAACCCCTTAAAGATAA |
| | MLV 10A1 | GATTAACCCGTGGAAGTCCTTAATGGTCATGGGGGTCT |
| | | ATTTAAGAGTAGGGATGGCAGAGAGCCCCCATCAGGTC |
| | | TTTAATGTAACCTGGAGAGTCACCAACCTGATGACTGG |
| | | GCGTACCGCCAATGCCACCTCCCTTTTAGGAACTGTAC |
| | | AAGATGCCTTCCCAAGATTATATTTTGATCTATGTGAT |
| | | CTGGTCGGAGAAGAGTGGGACCCTTCAGACCAGGAACC |
| | | ATATGTCGGGTATGGCTGCAAATACCCCGGAGGGAGAA |
| | | AGCGGACCCGGACTTTTGACTTTTACGTGTGCCCTGGG |
| | | CATACCGTAAAATCGGGGTGTGGGGGGCCAAGAGAGGG |
| | | CTACTGTGGTGAATGGGGTTGTGAAACCACCGGACAGG |
| | | CTTACTGGAAGCCCACATCATCATGGGACCTAATCTCC |
| | | CTTAAGCGCGGTAACACCCCCTGGGACACGGGATGCTC |
| | | CAAAATGGCTTGTGGCCCCTGCTACGACCTCTCCAAAG |
| | | TATCCAATTCCTTCCAAGGGGCTACTCGAGGGGGCAGA |
| | | TGCAACCCTCTAGTCCTAGAATTCACTGATGCAGGAAA |
| | | AAAGGCTAATTGGGACGGGCCCAAATCGTGGGGACTGA |
| | | GACTGTACCGGACAGGAACAGATCCTATTACCATGTTC |
| | | TCCCTGACCCGCCAGGTCCTCAATATAGGGCCCCGCAT |
| | | CCCCATTGGGCCTAATCCCGTGATCACTGGTCAACTAC |
| | | CCCCCTCCCGACCCGTGCAGATCAGGCTCCCCAGGCCT |
| | | CCTCAGCCTCCTCCTACAGGCGCAGCCTCTATAGTCCC |
| | | TGAGACTGCCCCACCTTCTCAACAACCTGGGACGGGAG |
| | | ACAGGCTGCTAAACCTGGTAGAAGGAGCCTATCAGGCG |
| | | CTTAACCTCACCAATCCCGACAAGACCCAAGAATGTTG |
| | | GCTGTGCTTAGTGTCGGGACCTCCTTATTACGAAGGAG |
| | | TAGCGGTCGTGGGCACTTATACCAATCATTCTACCGCC |
| | | CCGGCCAGCTGTACGGCCACTTCCCAACATAAGCTTAC |
| | | CCTATCTGAAGTGACAGGACAGGGCCTATGCATGGGAG |
| | | CACTACCTAAAACTCACCAGGCCTTATGTAACACCACC |
| | | CAAAGTGCCGGCTCAGGATCCTACTACCTTGCAGCACC |
| | | CGCTGGAACAATGTGGGCTTGTAGCACTGGATTGACTC |
| | | CCTGCTTGTCCACCACGATGCTCAATCTAACCACAGAC |
| | | TATTGTGTATTAGTTGAGCTCTGGCCCAGAATAATTTA |
| | | CCACTCCCCCGATTATATGTATGGTCAGCTTGAACAGC |
| | | GTACCAAATATAAGAGGGAGCCAGTATCGTTGACCCTG |
| | | GCCCTTCTGCTAGGAGGATTAACCATGGGAGGGATTGC |
| | | AGCTGGAATAGGGACGGGGACCACTGCCCTAATCAAAA |
| | | CCCAGCAGTTTGAGCAGCTTCACGCCGCTATCCAGACA |
| | | GACCTCAACGAAGTCGAAAAATCAATTACCAACCTAGA |
| | | AAAGTCACTGACCTCGTTGTCTGAAGTAGTCCTACAGA |
| | | ACCGAAGAGGCCTAGATTTGCTCTTCCTAAAAGAGGGA |
| | | GGTCTCTGCGCAGCCCTAAAAGAAGAATGTTGTTTTTA |
| | | TGCAGACCACACGGGACTAGTGAGAGACAGCATGGCCA |
| | | AACTAAGGGAAAGGCTTAATCAGAGACAAAAACTATTT |
| | | GAGTCAGGCCAAGGTTGGTTCGAAGGGCAGTTTAATAG |
| | | ATCCCCCTGGTTTACCACCTTAATCTCCACCATCATGG |
| | | GACCTCTAATAGTACTCTTACTGATCTTACTCTTTGGA |
| | | CCCTGCATTCTCAATCGATTGGTCCAATTTGTTAAAGA |
| | | CAGGATCTCAGTGGTCCAGGCTCTGGTTTTGACTCAAC |
| | | AATATCACCAGCTAAAACCTATAGAGTACGAGCCATGA |
| 82 | miR155 CD47 | CCTGGAGGCTTGCTGAAGGCTGTATGCTGTTATCCATC |
| | target sequence | TTCAAAGAGGCAGTTTTGGCCACTGACTGACTGCCTCT |
| | #1 | TAAGATGGATAACAGGACACAAGGCCTGTTACTAGCAC |
| | | TCA |
| 83 | miR21 cMyc | CATCTCCATGGCTGTACCACCTTGTCGGGTGTTCGCCT |
| | sequence | CTTGACATTCTCCTGTTGAATCTCATGGAGAATGTCAA |
| | | GGGCGAACACTGACATTTTGGTATCTTTCATCTGACCA |

While certain of the preferred embodiments of the present invention have been described and specifically exemplified above, it is not intended that the invention be limited to such embodiments. Various modifications may be made thereto without departing from the scope and spirit of the present invention.

What is claimed is:

1. A method of treating cancer in a subject using an immunotherapy-based composition, the method comprising administering to the subject a therapeutically effective amount of a composition, the composition comprising a lentiviral particle comprising:
an envelope protein optimized for infecting a target cell; and
a viral vector comprising a therapeutic cargo portion, wherein the therapeutic cargo portion comprises a small RNA sequence that is capable of binding to at least one pre-determined complementary mRNA sequence, wherein the at least one pre-determined complementary mRNA sequence comprises a FDPS mRNA sequence.


SEQUENCE LISTING (84 sequences)

SEQ ID NO: 1 - DNA, 53 bp, synthetic construct
  misc_feature 1..53: note = FDPS shRNA sequence #1
  source 1..53: mol_type = other DNA
gtcctggagtacaatgccattctcgagaatggcattgtactccaggacttttt

SEQ ID NO: 2 - DNA, 53 bp, synthetic construct
  misc_feature 1..53: note = FDPS shRNA sequence #2
  source 1..53: mol_type = other DNA
gcaggatttcgttcagcacttctcgagaagtgctgaacgaaatcctgcttttt

SEQ ID NO: 3 - DNA, 53 bp, synthetic construct
  misc_feature 1..53: note = FDPS shRNA sequence #3
  source 1..53: mol_type = other DNA
gccatgtacatggcaggaattctcgagaattcctgccatgtacatggcttttt

SEQ ID NO: 4 - DNA, 53 bp, synthetic construct
  misc_feature 1..53: note = FDPS shRNA sequence #4
  source 1..53: mol_type = other DNA
gcagaaggaggctgagaaagtctcgagactttctcagcctccttctgcttttt

SEQ ID NO: 5 - DNA, 49 bp, synthetic construct
  misc_feature 1..49: note = CD47 shRNA sequence #1
  source 1..49: mol_type = other DNA
ggtgaaacgatcatcgagcctcgaggctcgatgatcgtttcaccttttt

SEQ ID NO: 6 - DNA, 49 bp, synthetic construct
  misc_feature 1..49: note = CD47 shRNA sequence #2
  source 1..49: mol_type = other DNA
gctactggccttggtttaactcgagttaaaccaaggccagtagcttttt

SEQ ID NO: 7 - DNA, 49 bp, synthetic construct
  misc_feature 1..49: note = CD47 shRNA sequence #3
  source 1..49: mol_type = other DNA
cctccttcgtcattgccatctcgagatggcaatgacgaaggaggttttt

SEQ ID NO: 8 - DNA, 49 bp, synthetic construct
  misc_feature 1..49: note = CD47 shRNA sequence #4
  source 1..49: mol_type = other DNA
gcatggccctcttctgattctcgagaatcagaagagggccatgcttttt

SEQ ID NO: 9 - DNA, 53 bp, synthetic construct
  misc_feature 1..53: note = CD47 shRNA sequence #5
  source 1..53: mol_type = other DNA
ggtgaaacgatcatcgagctactcgagtagctcgatgatcgtttcaccttttt

SEQ ID NO: 10 - DNA, 52 bp, synthetic construct
  misc_feature 1..52: note = cMyc shRNA sequence #1
  source 1..52: mol_type = other DNA
gcttcaccaacaggaactatgctcgagcatagttcctgttggtgaagctttt

SEQ ID NO: 11 - DNA, 52 bp, synthetic construct
  misc_feature 1..52: note = cMyc shRNA sequence #2
  source 1..52: mol_type = other DNA
gcgaacacacaacgtcttggactcgagtccaagacgttgtgtgttcgctttt

SEQ ID NO: 12 - DNA, 53 bp, synthetic construct
  misc_feature 1..53: note = cMyc shRNA sequence #3
  source 1..53: mol_type = other DNA
gacatggtgaaccagagtttcctcgaggaaactctggttcaccatgtcttttt

SEQ ID NO: 13 - DNA, 53 bp, synthetic construct
  misc_feature 1..53: note = cMyc shRNA sequence #4
  source 1..53: mol_type = other DNA
gagaatgtcaagaggcgaacactcgagtgttcgcctcttgacattctcttttt

SEQ ID NO: 14 - DNA, 53 bp, synthetic construct
  misc_feature 1..53: note = cMyc shRNA sequence #5
  source 1..53: mol_type = other DNA
gctcatttctgaagaggacttctcgagaagtcctcttcagaaatgagcttttt

SEQ ID NO: 15 - DNA, 217 bp, synthetic construct
  misc_feature 1..217: note = H1 promoter
  source 1..217: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactt

SEQ ID NO: 16 - DNA, 249 bp, synthetic construct
  misc_feature 1..249: note = U6 promoter
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 17 - DNA, 243 bp, synthetic construct
  misc_feature 1..243: note = 7SK promoter
  source 1..243: mol_type = other DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagatttccttcaggtttatatagcttgtgcgccgcctggctacctc

SEQ ID NO: 18 - DNA, 352 bp, synthetic construct
  misc_feature 1..352: note = CAG enhancer
  source 1..352: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatc

SEQ ID NO: 19 - DNA, 290 bp, synthetic construct
  misc_feature 1..290: note = CAG promoter
  source 1..290: mol_type = other DNA
gctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 20 - DNA, 960 bp, synthetic construct
  misc_feature 1..960: note = chicken beta actin intron
  source 1..960: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgg

SEQ ID NO: 21 - DNA, 1503 bp, synthetic construct
  misc_feature 1..1503: note = HIV gag
  source 1..1503: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 22 - DNA, 1872 bp, synthetic construct
  misc_feature 1..1872: note = HIV Pol
  source 1..1872: mol_type = other DNA
atgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtacta

SEQ ID NO: 23 - DNA, 867 bp, synthetic construct
  misc_feature 1..867: note = HIV Int
  source 1..867: mol_type = other DNA
tttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 24 - DNA, 234 bp, synthetic construct
  misc_feature 1..234: note = HIV RRE
  source 1..234: mol_type = other DNA
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 25 - DNA, 351 bp, synthetic construct
  misc_feature 1..351: note = HIV Rev
  source 1..351: mol_type = other DNA
atggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatag

SEQ ID NO: 26 - DNA, 448 bp, synthetic construct
  misc_feature 1..448: note = rabbit beta globin poly A
  source 1..448: mol_type = other DNA
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatc

SEQ ID NO: 27 - DNA, 577 bp, synthetic construct
  misc_feature 1..577: note = CMV Promoter
  source 1..577: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 28 - DNA, 573 bp, synthetic construct
  misc_feature 1..573: note = beta globin intron
  source 1..573: mol_type = other DNA
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacag

SEQ ID NO: 29 - DNA, 1531 bp, synthetic construct
  misc_feature 1..1531: note = VSV-G / DNA fragment containing VSV-G
  source 1..1531: mol_type = other DNA
gaattcatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgagaattc

SEQ ID NO: 30 - DNA, 450 bp, synthetic construct
  misc_feature 1..450: note = rabbit beta globin poly A
  source 1..450: mol_type = other DNA
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 31 - DNA, 31 bp, synthetic construct
  misc_feature 1..31: note = Primer
  source 1..31: mol_type = other DNA
taagcagaattcatgaatttgccaggaagat

SEQ ID NO: 32 - DNA, 36 bp, synthetic construct
  misc_feature 1..36: note = Primer
  source 1..36: mol_type = other DNA
ccatacaatgaatggacactaggcggccgcacgaat

SEQ ID NO: 33 - DNA, 2745 bp, synthetic construct
  misc_feature 1..2745: note = Gag, Pol, Integrase fragment
  source 1..2745: mol_type = other DNA
gaattcatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 34 - DNA, 1586 bp, synthetic construct
  misc_feature 1..1586: note = DNA Fragment containing Rev, RRE and rabbit beta globin poly A
  source 1..1586: mol_type = other DNA
tctagaatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcagcggccgccccggg

SEQ ID NO: 35 - DNA, 1614 bp, synthetic construct
  misc_feature 1..1614: note = DNA fragment containing the CAG enhancer/promoter/intron sequence
  source 1..1614: mol_type = other DNA
acgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggaattc

SEQ ID NO: 36 - DNA, 884 bp, synthetic construct
  misc_feature 1..884: note = RSV promoter and HIV Rev
  source 1..884: mol_type = other DNA
caattgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagcgattaggcatctcctatggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtctaga

SEQ ID NO: 37 - DNA, 1104 bp, synthetic construct
  misc_feature 1..1104: note = Elongation Factor-1 alpha (EF1-alpha) promoter
  source 1..1104: mol_type = other DNA
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 38 - DNA, 511 bp, synthetic construct
  misc_feature 1..511: note = Promoter - PGK
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 39 - DNA, 1162 bp, synthetic construct
  misc_feature 1..1162: note = Promoter - UbC
  source 1..1162: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 40 - DNA, 120 bp, synthetic construct
  misc_feature 1..120: note = Poly A - SV40
  source 1..120: mol_type = other DNA
gtttattgcagcttataatggttacaaataaagcaatagcatcacaaatttcacaaataaagcatttttttcactgcattctagttgtggtttgtccaaactcatcaatgtatcttatca

SEQ ID NO: 41 - DNA, 227 bp, synthetic construct
  misc_feature 1..227: note = Poly A - bGH
  source 1..227: mol_type = other DNA
gactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 42 - DNA, 1695 bp, synthetic construct
  misc_feature 1..1695: note = Envelope - RD114
  source 1..1695: mol_type = other DNA
atgaaactcccaacaggaatggtcattttatgtagcctaataatagttcgggcagggtttgacgacccccgcaaggctatcgcattagtacaaaaacaacatggtaaaccatgcgaatgcagcggagggcaggtatccgaggccccaccgaactccatccaacaggtaacttgcccaggcaagacggcctacttaatgaccaaccaaaaatggaaatgcagagtcactccaaaaaatctcacccctagcgggggagaactccagaactgcccctgtaacactttccaggactcgatgcacagttcttgttatactgaataccggcaatgcagggcgaataataagacatactacacggccaccttgcttaaaatacggtctgggagcctcaacgaggtacagatattacaaaaccccaatcagctcctacagtccccttgtaggggctctataaatcagcccgtttgctggagtgccacagcccccatccatatctccgatggtggaggacccctcgatactaagagagtgtggacagtccaaaaaaggctagaacaaattcataaggctatgcatcctgaacttcaataccaccccttagccctgcccaaagtcagagatgaccttagccttgatgcacggacttttgatatcctgaataccacttttaggttactccagatgtccaattttagccttgcccaagattgttggctctgtttaaaactaggtacccctacccctcttgcgatacccactccctctttaacctactccctagcagactccctagcgaatgcctcctgtcagattatacctcccctcttggttcaaccgatgcagttctccaactcgtcctgtttatcttcccctttcattaacgatacggaacaaatagacttaggtgcagtcacctttactaactgcacctctgtagccaatgtcagtagtcctttatgtgccctaaacgggtcagtcttcctctgtggaaataacatggcatacacctatttaccccaaaactggacaggactttgcgtccaagcctccctcctccccgacattgacatcatcccgggggatgagccagtccccattcctgccattgatcattatatacatagacctaaacgagctgtacagttcatccctttactagctggactgggaatcaccgcagcattcaccaccggagctacaggcctaggtgtctccgtcacccagtatacaaaattatcccatcagttaatatctgatgtccaagtcttatccggtaccatacaagatttacaagaccaggtagactcgttagctgaagtagttctccaaaataggaggggactggacctactaacggcagaacaaggaggaatttgtttagccttacaagaaaaatgctgtttttatgctaacaagtcaggaattgtgagaaacaaaataagaaccctacaagaagaattacaaaaacgcagggaaagcctggcatccaaccctctctggaccgggctgcagggctttcttccgtacctcctacctctcctgggacccctactcaccctcctactcatactaaccattgggccatgcgttttcaatcgattggtccaatttgttaaagacaggatctcagtggtccaggctctggttttgactcagcaatatcaccagctaaaacccatagagtacgagccatga

SEQ ID NO: 43 - DNA, 2013 bp, synthetic construct
  misc_feature 1..2013: note = Envelope - GALV
  source 1..2013: mol_type = other DNA
atgcttctcacctcaagcccgcaccaccttcggcaccagatgagtcctgggagctggaaaagactgatcatcctcttaagctgcgtattcggagacggcaaaacgagtctgcagaataagaacccccaccagcctgtgaccctcacctggcaggtactgtcccaaactggggacgttgtctgggacaaaaaggcagtccagcccctttggacttggtggccctctcttacacctgatgtatgtgccctggcggccggtcttgagtcctgggatatcccgggatccgatgtatcgtcctctaaaagagttagacctcctgattcagactatactgccgcttataagcaaatcacctggggagccatagggtgcagctaccctcgggctaggaccaggatggcaaattcccccttctacgtgtgtccccgagctggccgaacccattcagaagctaggaggtgtggggggctagaatccctatactgtaaagaatggagttgtgagaccacgggtaccgtttattggcaacccaagtcctcatgggacctcataactgtaaaatgggaccaaaatgtgaaatgggagcaaaaatttcaaaagtgtgaacaaaccggctggtgtaaccccctcaagatagacttcacagaaaaaggaaaactctccagagattggataacggaaaaaacctgggaattaaggttctatgtatatggacacccaggcatacagttgactatccgcttagaggtcactaacatgccggttgtggcagtgggcccagaccctgtccttgcggaacagggacctcctagcaagcccctcactctccctctctccccacggaaagcgccgcccacccctctacccccggcggctagtgagcaaacccctgcggtgcatggagaaactgttaccctaaactctccgcctcccaccagtggcgaccgactctttggccttgtgcagggggccttcctaaccttgaatgctaccaacccaggggccactaagtcttgctggctctgtttgggcatgagccccccttattatgaagggatagcctcttcaggagaggtcgcttatacctccaaccatacccgatgccactggggggcccaaggaaagcttaccctcactgaggtctccggactcgggtcatgcatagggaaggtgcctcttacccatcaacatctttgcaaccagaccttacccatcaattcctctaaaaaccatcagtatctgctcccctcaaaccatagctggtgggcctgcagcactggcctcaccccctgcctctccacctcagtttttaatcagtctaaagacttctgtgtccaggtccagctgatcccccgcatctattaccattctgaagaaaccttgttacaagcctatgacaaatcaccccccaggtttaaaagagagcctgcctcacttaccctagctgtcttcctggggttagggattgcggcaggtataggtactggctcaaccgccctaattaaagggcccatagacctccagcaaggcctaaccagcctccaaatcgccattgacgctgacctccgggcccttcaggactcaatcagcaagctagaggactcactgacttccctatctgaggtagtactccaaaataggagaggccttgacttactattccttaaagaaggaggcctctgcgcggccctaaaagaagagtgctgtttttatgtagaccactcaggtgcagtacgagactccatgaaaaaacttaaagaaagactagataaaagacagttagagcgccagaaaaaccaaaactggtatgaagggtggttcaataactccccttggtttactaccctactatcaaccatcgctgggcccctattgctcctccttttgttactcactcttgggccctgcatcatcaataaattaatccaattcatcaatgataggataagtgcagtcaaaattttagtccttagacagaaatatcagaccctagataacgaggaaaacctttaa

SEQ ID NO: 44 - DNA, 1530 bp, synthetic construct
  misc_feature 1..1530: note = Envelope - FUG
  source 1..1530: mol_type = other DNA
atggttccgcaggttcttttgtttgtactccttctgggtttttcgttgtgtttcgggaagttccccatttacacgataccagacgaacttggtccctggagccctattgacatacaccatctcagctgtccaaataacctggttgtggaggatgaaggatgtaccaacctgtccgagttctcctacatggaactcaaagtgggatacatctcagccatcaaagtgaacgggttcacttgcacaggtgttgtgacagaggcagagacctacaccaactttgttggttatgtcacaaccacattcaagagaaagcatttccgccccaccccagacgcatgtagagccgcgtataactggaagatggccggtgaccccagatatgaagagtccctacacaatccataccccgactaccactggcttcgaactgtaagaaccaccaaagagtccctcattatcatatccccaagtgtgacagatttggacccatatgacaaatcccttcactcaagggtcttccctggcggaaagtgctcaggaataacggtgtcctctacctactgctcaactaaccatgattacaccatttggatgcccgagaatccgagaccaaggacaccttgtgacatttttaccaatagcagagggaagagagcatccaacgggaacaagacttgcggctttgtggatgaaagaggcctgtataagtctctaaaaggagcatgcaggctcaagttatgtggagttcttggacttagacttatggatggaacatgggtcgcgatgcaaacatcagatgagaccaaatggtgccctccagatcagttggtgaatttgcacgactttcgctcagacgagatcgagcatctcgttgtggaggagttagttaagaaaagagaggaatgtctggatgcattagagtccatcatgaccaccaagtcagtaagtttcagacgtctcagtcacctgagaaaacttgtcccagggtttggaaaagcatataccatattcaacaaaaccttgatggaggctgatgctcactacaagtcagtccggacctggaatgagatcatcccctcaaaagggtgtttgaaagttggaggaaggtgccatcctcatgtgaacggggtgtttttcaatggtataatattagggcctgacgaccatgtcctaatcccagagatgcaatcatccctcctccagcaacatatggagttgttggaatcttcagttatccccctgatgcaccccctggcagacccttctacagttttcaaagaaggtgatgaggctgaggattttgttgaagttcacctccccgatgtgtacaaacagatctcaggggttgacctgggtctcccgaactggggaaagtatgtattgatgactgcaggggccatgattggcctggtgttgatattttccctaatgacatggtgcagagttggtatccatctttgcattaaattaaagcacaccaagaaaagacagatttatacagacatagagatgaaccgacttggaaagtaa

SEQ ID NO: 45 - DNA, 1497 bp, synthetic construct
  misc_feature 1..1497: note = Envelope - LCMV
  source 1..1497: mol_type = other DNA
atgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctga

SEQ ID NO: 46 - DNA, 1692 bp, synthetic construct
  misc_feature 1..1692: note = Envelope - FPV
  source 1..1692: mol_type = other DNA
atgaacactcaaatcctggttttcgcccttgtggcagtcatccccacaaatgcagacaaaatttgtcttggacatcatgctgtatcaaatggcaccaaagtaaacacactcactgagagaggagtagaagttgtcaatgcaacggaaacagtggagcggacaaacatccccaaaatttgctcaaaagggaaaagaaccactgatcttggccaatgcggactgttagggaccattaccggaccacctcaatgcgaccaatttctagaattttcagctgatctaataatcgagagacgagaaggaaatgatgtttgttacccggggaagtttgttaatgaagaggcattgcgacaaatcctcagaggatcaggtgggattgacaaagaaacaatgggattcacatatagtggaataaggaccaacggaacaactagtgcatgtagaagatcagggtcttcattctatgcagaaatggagtggctcctgtcaaatacagacaatgctgctttcccacaaatgacaaaatcatacaaaaacacaaggagagaatcagctctgatagtctggggaatccaccattcaggatcaaccaccgaacagaccaaactatatgggagtggaaataaactgataacagtcgggagttccaaatatcatcaatcttttgtgccgagtccaggaacacgaccgcagataaatggccagtccggacggattgattttcattggttgatcttggatcccaatgatacagttacttttagtttcaatggggctttcatagctccaaatcgtgccagcttcttgaggggaaagtccatggggatccagagcgatgtgcaggttgatgccaattgcgaaggggaatgctaccacagtggagggactataacaagcagattgccttttcaaaacatcaatagcagagcagttggcaaatgcccaagatatgtaaaacaggaaagtttattattggcaactgggatgaagaacgttcccgaaccttccaaaaaaaggaaaaaaagaggcctgtttggcgctatagcagggtttattgaaaatggttgggaaggtctggtcgacgggtggtacggtttcaggcatcagaatgcacaaggagaaggaactgcagcagactacaaaagcacccaatcggcaattgatcagataaccggaaagttaaatagactcattgagaaaaccaaccagcaatttgagctaatagataatgaattcactgaggtggaaaagcagattggcaatttaattaactggaccaaagactccatcacagaagtatggtcttacaatgctgaacttcttgtggcaatggaaaaccagcacactattgatttggctgattcagagatgaacaagctgtatgagcgagtgaggaaacaattaagggaaaatgctgaagaggatggcactggttgctttgaaatttttcataaatgtgacgatgattgtatggctagtataaggaacaatacttatgatcacagcaaatacagagaagaagcgatgcaaaatagaatacaaattgacccagtcaaattgagtagtggctacaaagatgtgatactttggtttagcttcggggcatcatgctttttgcttcttgccattgcaatgggccttgttttcatatgtgtgaagaacggaaacatgcggtgcactatttgtatataa

SEQ ID NO: 47 - DNA, 1266 bp, synthetic construct
  misc_feature 1..1266: note = Envelope - RRV
  source 1..1266: mol_type = other DNA
agtgtaacagagcactttaatgtgtataaggctactagaccatacctagcacattgcgccgattgcggggacgggtacttctgctatagcccagttgctatcgaggagatccgagatgaggcgtctgatggcatgcttaagatccaagtctccgcccaaataggtctggacaaggcaggcacccacgcccacacgaagctccgatatatggctggtcatgatgttcaggaatctaagagagattccttgagggtgtacacgtccgcagcgtgctccatacatgggacgatgggacacttcatcgtcgcacactgtccaccaggcgactacctcaaggtttcgttcgaggacgcagattcgcacgtgaaggcatgtaaggtccaatacaagcacaatccattgccggtgggtagagagaagttcgtggttagaccacactttggcgtagagctgccatgcacctcataccagctgacaacggctcccaccgacgaggagattgacatgcatacaccgccagatataccggatcgcaccctgctatcacagacggcgggcaacgtcaaaataacagcaggcggcaggactatcaggtacaactgtacctgcggccgtgacaacgtaggcactaccagtactgacaagaccatcaacacatgcaagattgaccaatgccatgctgccgtcaccagccatgacaaatggcaatttacctctccatttgttcccagggctgatcagacagctaggaaaggcaaggtacacgttccgttccctctgactaacgtcacctgccgagtgccgttggctcgagcgccggatgccacctatggtaagaaggaggtgaccctgagattacacccagatcatccgacgctcttctcctataggagtttaggagccgaaccgcacccgtacgaggaatgggttgacaagttctctgagcgcatcatcccagtgacggaagaagggattgagtaccagtggggcaacaacccgccggtctgcctgtgggcgcaactgacgaccgagggcaaaccccatggctggccacatgaaatcattcagtactattatggactataccccgccgccactattgccgcagtatccggggcgagtctgatggccctcctaactctggcggccacatgctgcatgctggccaccgcgaggagaaagtgcctaacaccgtacgccctgacgccaggagcggtggtaccgttgacactggggctgctttgctgcgcaccgagggcgaatgca

SEQ ID NO: 48 - DNA, 2030 bp, synthetic construct
  misc_feature 1..2030: note = Envelope - Ebola
  source 1..2030: mol_type = other DNA
atgggtgttacaggaatattgcagttacctcgtgatcgattcaagaggacatcattctttctttgggtaattatccttttccaaagaacattttccatcccacttggagtcatccacaatagcacattacaggttagtgatgtcgacaaactggtttgccgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccacaaagagggtgctttcttcctgtatgaccgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaagctaccggttttggaaccaatgagacagagtatttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagctgtatcaaacagagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctgacaacccttgccacaatctccacgagtcctcaaccccccacaaccaaaccaggtccggacaacagcacccacaatacacccgtgtataaacttgacatctctgaggcaactcaagttgaacaacatcaccgcagaacagacaacgacagcacagcctccgacactccccccgccacgaccgcagccggacccctaaaagcagagaacaccaacacgagcaagggtaccgacctcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggaggagagctcgaagagaagcaattgtcaatgctcaacccaaatgcaaccctaatttacattactggactactcaggatgaaggtgctgcaatcggactggcctggataccatatttcgggccagcagccgagggaatttacatagaggggctgatgcacaatcaagatggtttaatctgtgggttgagacagctggccaacgagacgactcaagctcttcaactgttcctgagagccacaaccgagctacgcaccttttcaatcctcaaccgtaaggcaattgatttcttgctgcagcgatggggcggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 49 - DNA, 21 bp, synthetic construct
  misc_feature 1..21: note = FDPS target sequence #1
  source 1..21: mol_type = other DNA
gtcctggagtacaatgccatt

SEQ ID NO: 50 - DNA, 21 bp, synthetic construct
  misc_feature 1..21: note = FDPS target sequence #2
  source 1..21: mol_type = other DNA
gcaggatttcgttcagcactt

SEQ ID NO: 51 - DNA, 21 bp, synthetic construct
  misc_feature 1..21: note = FDPS target sequence #3
  source 1..21: mol_type = other DNA
gccatgtacatggcaggaatt

SEQ ID NO: 52 - DNA, 21 bp, synthetic construct
  misc_feature 1..21: note = FDPS target sequence #4
  source 1..21: mol_type = other DNA
gcagaaggaggctgagaaagt

SEQ ID NO: 53 - DNA, 116 bp, synthetic construct
  misc_feature 1..116: note = miR30 FDPS sequence #1
  source 1..116: mol_type = other DNA
aaggtatattgctgttgacagtgagcgacactttctcagcctccttctgcgtgaagccacagatggcagaaggaggctgagaaagtgctgcctactgcctcggacttcaaggggct

SEQ ID NO: 54 - DNA, 114 bp, synthetic construct
  misc_feature 1..114: note = miR30 FDPS sequence #2
  source 1..114: mol_type = other DNA
aaggtatattgctgttgacagtgagcgacactttctcagcctccttctgcgtgaagccacagatggcagaagggctgagaaagtgctgcctactgcctcggacttcaaggggct

SEQ ID NO: 55 - DNA, 91 bp, synthetic construct
  misc_feature 1..91: note = miR30 FDPS sequence #3
  source 1..91: mol_type = other DNA
tgctgttgacagtgagcgactttctcagcctccttctgcgtgaagccacagatggcagaaggaggctgagaaagttgcctactgcctcgga

SEQ ID NO: 56 - DNA, 115 bp, synthetic construct
  misc_feature 1..115: note = miR155 FDPS sequence #1
  source 1..115: mol_type = other DNA
cctggaggcttgctgaaggctgtatgctgactttctcagcctccttctgcttttggccactgactgagcagaagggctgagaaagtcaggacacaaggcctgttactagcactca

SEQ ID NO: 57 - DNA, 114 bp, synthetic construct
  misc_feature 1..114: note = miR21 FDPS sequence #1
  source 1..114: mol_type = other DNA
catctccatggctgtaccaccttgtcgggactttctcagcctccttctgcctgttgaatctcatggcagaaggaggcgagaaagtctgacattttggtatctttcatctgacca

SEQ ID NO: 58 - DNA, 114 bp, synthetic construct
  misc_feature 1..114: note = miR185 FDPS sequence #1
  source 1..114: mol_type = other DNA
gggcctggctcgagcagggggcgagggatactttctcagcctccttctgctggtcccctccccgcagaaggaggctgagaaagtccttccctcccaatgaccgcgtcttcgtcg

SEQ ID NO: 59 - DNA, 20 bp, synthetic construct
  misc_feature 1..20: note = Forward Primer
  source 1..20: mol_type = other DNA
aggaattgatggcgagaagg

SEQ ID NO: 60 - DNA, 22 bp, synthetic construct
  misc_feature 1..22: note = Reverse Primer
  source 1..22: mol_type = other DNA
cccaaagaggtcaaggtaatca

SEQ ID NO: 61 - DNA, 18 bp, synthetic construct
  misc_feature 1..18: note = Forward Primer
  source 1..18: mol_type = other DNA
agcgcggctacagcttca

SEQ ID NO: 62 - DNA, 20 bp, synthetic construct
  misc_feature 1..20: note = Reverse Primer
  source 1..20: mol_type = other DNA
ggcgacgtagcacagcttct

SEQ ID NO: 63 - DNA, 20 bp, synthetic construct
  misc_feature 1..20: note = Forward Primer
  source 1..20: mol_type = other DNA
cactgtcgtcattccatgct

SEQ ID NO: 64 - DNA, 19 bp, synthetic construct
  misc_feature 1..19: note = Reverse Primer
  source 1..19: mol_type = other DNA
gcctcttgacattctcctc

SEQ ID NO: 65 - DNA, 20 bp, synthetic construct
  misc_feature 1..20: note = Reverse Primer
  source 1..20: mol_type = other DNA
aaagtcagtggggacagtgg

SEQ ID NO: 66 - DNA, 117 bp, synthetic construct
  misc_feature 1..117: note = miR155 CD47 target sequence #2
  source 1..117: mol_type = other DNA
cctggaggcttgctgaaggctgtatgctgttagctcgatgatcgtttcacgttttggccactgactgacgtgaaacgcatcgagctaacaggacacaaggcctgttactagcactca

SEQ ID NO: 67 - DNA, 117 bp, synthetic construct
  misc_feature 1..117: note = miR155 CD47 target sequence #3
  source 1..117: mol_type = other DNA
cctggaggcttgctgaaggctgtatgctgaagaatggctccaacaatgacgttttggccactgactgacgtcattgtgagccattcttcaggacacaaggcctgttactagcactca

SEQ ID NO: 68 - DNA, 117 bp, synthetic construct
  misc_feature 1..117: note = miR155 CD47 target sequence #4
  source 1..117: mol_type = other DNA
cctggaggcttgctgaaggctgtatgctgtatacacgccgcaatacagaggttttggccactgactgacctctgtatcggcgtgtatacaggacacaaggcctgttactagcactca

SEQ ID NO: 69 - DNA, 18 bp, synthetic construct
  misc_feature 1..18: note = Forward primer
  source 1..18: mol_type = other DNA
ggactatcctgctgccaa

SEQ ID NO: 70 - DNA, 115 bp, synthetic construct
  misc_feature 1..115: note = miR155 cMyc sequence
  source 1..115: mol_type = other DNA
cctggaggcttgctgaaggctgtatgctgtgttcgcctcttgacattctcttttggccactgactgagagaatgtagaggcgaacacaggacacaaggcctgttactagcactca

SEQ ID NO: 71 - DNA, 21 bp, synthetic construct
  misc_feature 1..21: note = cMyc target sequence
  source 1..21: mol_type = other DNA
gagaatgtcaagaggcgaaca

SEQ ID NO: 72 - DNA, 329 bp, synthetic construct
  misc_feature 1..329: note = CMV promoter sequence
  source 1..329: mol_type = other DNA
attatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgtttt

SEQ ID NO: 73 - DNA, 2520 bp, synthetic construct
  misc_feature 1..2520: note = GFP T2A Luciferase sequence
  source 1..2520: mol_type = other DNA
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagatatcagccatggcttcccgccggcggtggcggcgcaggatgatggcacgctgcccatgtcttgtgcccaggagagcgggatggaccgtcaccctgcagcctgtgcttctgctaggatcaatgtgaccggtgagggcagaggaagtcttctaacatgcggtgacgtggaggagaatcccggcccttccggtatggaagacgccaaaaacataaagaaaggcccggcgccattctatccgctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 74 - DNA, 228 bp, synthetic construct
  misc_feature 1..228: note = Rous Sarcoma virus (RSV) promoter
  source 1..228: mol_type = other DNA
gtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacg

SEQ ID NO: 75 - DNA, 180 bp, synthetic construct
  misc_feature 1..180: note = 5' Long terminal repeat (LTR)
  source 1..180: mol_type = other DNA
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 76 - DNA, 41 bp, synthetic construct
  misc_feature 1..41: note = Psi Packaging signal
  source 1..41: mol_type = other DNA
tacgccaaaaattttgactagcggaggctagaaggagagag

SEQ ID NO: 77 - DNA, 233 bp, synthetic construct
  misc_feature 1..233: note = Rev response element (RRE)
  source 1..233: mol_type = other DNA
aggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcc

SEQ ID NO: 78 - DNA, 118 bp, synthetic construct
  misc_feature 1..118: note = Central polypurine tract (cPPT)
  source 1..118: mol_type = other DNA
ttttaaaagaaaaggggggattggggggtacagtgcaggggaaagaatagtagacataatagcaacagacatacaaactaaagaattacaaaaacaaattacaaaattcaaaatttta

SEQ ID NO: 79 - DNA, 590 bp, synthetic construct
  misc_feature 1..590: note = Long WPRE sequence
  source 1..590: mol_type = other DNA
aatcaacctctgattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 80 - DNA, 250 bp, synthetic construct
  misc_feature 1..250: note = 3' delta LTR
  source 1..250: mol_type = other DNA
tggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtca

SEQ ID NO: 81 - DNA, 1938 bp, synthetic construct
  misc_feature 1..1938: note = Envelope - MLV 10A1
  source 1..1938: mol_type = other DNA
atggaaggtccagcgttctcaaaaccccttaaagataagattaacccgtggaagtccttaatggtcatgggggtctatttaagagtagggatggcagagagcccccatcaggtctttaatgtaacctggagagtcaccaacctgatgactgggcgtaccgccaatgccacctcccttttaggaactgtacaagatgccttcccaagattatattttgatctatgtgatctggtcggagaagagtgggacccttcagaccaggaaccatatgtcgggtatggctgcaaataccccggagggagaaagcggacccggacttttgacttttacgtgtgccctgggcataccgtaaaatcggggtgtggggggccaagagagggctactgtggtgaatggggttgtgaaaccaccggacaggcttactggaagcccacatcatcatgggacctaatctcccttaagcgcggtaacaccccctgggacacgggatgctccaaaatggcttgtggcccctgctacgacctctccaaagtatccaattccttccaaggggctactcgagggggcagatgcaaccctctagtcctagaattcactgatgcaggaaaaaaggctaattgggacgggcccaaatcgtggggactgagactgtaccggacaggaacagatcctattaccatgttctccctgacccgccaggtcctcaatatagggccccgcatccccattgggcctaatcccgtgatcactggtcaactacccccctcccgacccgtgcagatcaggctccccaggcctcctcagcctcctcctacaggcgcagcctctatagtccctgagactgccccaccttctcaacaacctgggacgggagacaggctgctaaacctggtagaaggagcctatcaggcgcttaacctcaccaatcccgacaagacccaagaatgttggctgtgcttagtgtcgggacctccttattacgaaggagtagcggtcgtgggcacttataccaatcattctaccgccccggccagctgtacggccacttcccaacataagcttaccctatctgaagtgacaggacagggcctatgcatgggagcactacctaaaactcaccaggccttatgtaacaccacccaaagtgccggctcaggatcctactaccttgcagcacccgctggaacaatgtgggcttgtagcactggattgactccctgcttgtccaccacgatgctcaatctaaccacagactattgtgtattagttgagctctggcccagaataatttaccactcccccgattatatgtatggtcagcttgaacagcgtaccaaatataagagggagccagtatcgttgaccctggcccttctgctaggaggattaaccatgggagggattgcagctggaatagggacggggaccactgccctaatcaaaacccagcagtttgagcagcttcacgccgctatccagacagacctcaacgaagtcgaaaaatcaattaccaacctagaaaagtcactgacctcgttgtctgaagtagtcctacagaaccgaagaggcctagatttgctcttcctaaaagagggaggtctctgcgcagccctaaaagaagaatgttgtttttatgcagaccacacgggactagtgagagacagcatggccaaactaagggaaaggcttaatcagagacaaaaactatttgagtcaggccaaggttggttcgaagggcagtttaatagatccccctggtttaccaccttaatctccaccatcatgggacctctaatagtactcttactgatcttactctttggaccctgcattctcaatcgattggtccaatttgttaaagacaggatctcagtggtccaggctctggttttgactcaacaatatcaccagctaaaacctatagagtacgagccatga

SEQ ID NO: 82 - DNA, 117 bp, synthetic construct
  misc_feature 1..117: note = miR155 CD47 target sequence #1
  source 1..117: mol_type = other DNA
cctggaggcttgctgaaggctgtatgctgttatccatcttcaaagaggcagttttggccactgactgactgcctcttaagatggataacaggacacaaggcctgttactagcactca

SEQ ID NO: 83 - DNA, 114 bp, synthetic construct
  misc_feature 1..114: note = miR21 cMyc sequence
  source 1..114: mol_type = other DNA
catctccatggctgtaccaccttgtcgggtgttcgcctcttgacattctcctgttgaatctcatggagaatgtcaagggcgaacactgacattttggtatctttcatctgacca

SEQ ID NO: 84 - DNA, 1227 bp, synthetic construct
  misc_feature 1..1227: note = Helper plasmid without Rev
  source 1..1227: mol_type = other DNA
tctagaaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcacccggg